(19) United States
(12) Patent Application Publication
(10) Pub. No.: US 2023/0287416 A1
(43) Pub. Date: Sep. 14, 2023

(54) COMBINATORY TREATMENT OF SMA WITH SARNA AND MRNA MODULATORS

(71) Applicant: RACTIGEN THERAPEUTICS, Jiangsu (CN)

(72) Inventors: Longcheng LI, Jiangsu (CN); Moorim KANG, Jiangsu (CN); Jiancheng WU, Jiangsu (CN)

(21) Appl. No.: 18/007,497

(22) PCT Filed: Jul. 29, 2021
(86) PCT No.: PCT/CN2021/109146

§ 371 Date: Jan. 31, 2023

(30) Foreign Application Priority Data
Jul. 31, 2020 (WO) PCT/CN2020/106200

(51) Int. Cl. C12N 15/113; A61K 31/519; A61K 31/501

(52) U.S. Cl. CPC C12N 15/113; A61K 31/519; A61K 31/501; C12N 2320/31; C12N 2320/33; C12N 2310/11

(57) ABSTRACT

Provided herein are methods and compositions related to combinations of (a) agents that increase the expression of SMN2 gene or protein, and (b) modulators of SMN2 mRNA splicing or stability that increase the production of functional SMN2 mRNA and SMN protein, and their use in treating SMA and related conditions or diseases. In certain embodiments, the methods relate to using SMN2 saRNA and SMN2 mRNA modulators for diminishing the symptoms of SMA.

### 1. CROSS-REFERENCE

This application is the U.S. National Stage of International Patent Application No. PCT/CN2021/109146, filed Jul. 29, 2021, which claims the benefit of PCT Patent Application No. PCT/CN2020/106200, filed Jul. 31, 2020, each of which is incorporated herein by reference in its entirety.

### 2. INCORPORATION BY REFERENCE OF SEQUENCE LISTING PROVIDED AS A TEXT FILE

A Sequence Listing is provided herewith as an ASCII compliant computer readable text file entitled “SequenceListing58403.txt” created on Mar. 27, 2023, with a file size of 102,309 bytes. The contents of the text file are incorporated by reference herein in their entirety.

### 3. BACKGROUND OF THE INVENTION

Spinal muscular atrophy (SMA) is an autosomal recessive disorder affecting approximately 1 in 6000-8000 newborns and is the leading hereditary cause of mortality in infants. SMA is caused by reduced levels of survival motor neuron (SMN) protein as a result of a homozygous deletion or mutation of the telomeric copy of the survival of motor neuron gene (SMN1) on chromosome 5q13.4.

The SMN protein is encoded by two SMN genes (SMN1 and SMN2), which essentially differ in their coding sequence by one nucleotide in exon 7 in that a cytosine (C) is changed to a thymine (T) in SMN2 gene (Coovert, D. D., et al. The survival motor neuron protein in spinal muscular atrophy. Human Mol Genet (1997)). This critical difference creates a cryptic splicing site and leads to exon 7 skipping in ˜90% of mature SMN mRNA transcribed from SMN2 gene. SMN2 mRNA lacking exon 7 (SMN2 Δ7) gives rise to a truncated SMN protein that is unstable and rapidly degraded. In SMA patients, the SMN1 gene no longer produces any SMN protein, and the amount of full length SMN protein produced by SMN2 is not sufficient to compensate for the loss of SMN1, leading to the apoptotic death of the motor neuron in the anterior horn of the spinal cord, atrophy of skeletal muscles, and consequent weakness (Monani, U. R., et al. The human centromeric survival motor neuron gene (SMN2) rescues embryonic lethality in Smn(−/−) mice and results in a mouse with spinal muscular atrophy. Human Mol Genet (2000)). The severity of the symptoms for SMA patients depends on the copy number of the SMN2 gene in a patient's cells—a larger number of copies results in less severe symptoms (Harada, Y., et al. Correlation between SMN2 copy number and clinical phenotype of spinal muscular atrophy: three SMN2 copies fail to rescue some patients from the disease severity. J Neurol (2002)).

To develop therapeutics for SMA, one strategy is to use splicing modulators (SMs) to stimulate exon 7 inclusion during the splicing process. In this regard, an antisense oligonucleotide (ASO) drug Spinraza® has already been approved for marketing by the U.S. Food and Drug Administration (FDA) (Hua, Y. and A. R. Krainer. Antisense-mediated exon inclusion. Methods Mol Biol (2012) and Stein, C. A. and D. Castanotto. FDA-Approved Oligonucleotide Therapies in 2017. Mol Thera (2017)). Another drug, Risdiplam (RG7916), an investigational, orally administered small molecule drug, is under New Drug Application (NDA) with the FDA (Ramdas, S. and L. Servais. New treatments in spinal muscular atrophy: an overview of currently available data. Expert Opin Pharmaco (2020)). These two drugs have changed the treatment of SMA by significantly extending patients' survival and improving motor milestones. Despite those improvements, treated patients, especially children, are far from living normal lives. Several plausible reasons could explain the inadequate efficacy of SMs. One is the ceiling effect that limits the maximum achievable level of full-length SMN protein restored by the treatments. SMs do not have an effect on SMN2 transcription, and so do not increase the amount of available SMN2 pre-mRNA. To restore SMN protein to its normal physiological level, SMs optimally would achieve a 100% in vivo efficiency in converting SMN2 Δ7 mRNA to full-length mRNA, an ideal effect that is unlikely to occur in reality. Thus, the maximal efficacy SMs can offer to patients is limited by the availability of SMN2 pre-mRNA.

Another therapeutic approach to treating SMA involves stimulating SMN2 transcription to increase the levels of full-length SMN protein. Previously, various epigenetic modifying agents, such as histone deacetylase (HDAC) inhibitors (e.g., sodium butyrate, valproic acid) and non HDAC inhibitors (e.g., hydroxyurea, celecoxib, albuterol, etc.), have been tested in vitro and in mouse SMA models, but these failed to demonstrate noticeable clinical efficacy (Lunke, S. and A. El-Osta. The emerging role of epigenetic modifications and chromatin remodeling in spinal muscular atrophy. J Neurochem (2009)). An explanation for the failure is the lack of target specificity of epigenetic modifying agents. There exists a need for improved methods and compositions for treating SMN-deficiency-related conditions, such as spinal muscular atrophy.

### 4. SUMMARY OF THE INVENTION

Double-stranded RNAs (dsRNAs) targeting gene regulatory sequences, including promoters, have been shown to upregulate target genes in a sequence-specific manner at the transcriptional level via a mechanism known as RNA activation (RNAa) (Li, L. C., et al. Small dsRNAs induce transcriptional activation in human cells. PNAS (2006)). Such dsRNAs are termed small activating RNAs (saRNAs). A prior patent publication (PCT/CN2019/129025), incorporated herein by reference in its entirety, described the identification of functional saRNAs targeting the SMN2 promoter and described their activity in inducing SMN2 mRNA expression in both normal human cells and primary cells derived from SMA patients.

Embodiments of the present disclosure are based in part on the surprising discovery that a combination of (a) one or more small activating ribonucleic acids (saRNAs) that activate or upregulate the expression of an SMN2 gene (also referred to as “SMN2 saRNAs” herein) in a cell, and (b) one or more modulators of SMN2 mRNA splicing or stability (also referred to as “SMN2 mRNA modulators” herein) that increase the production of functional SMN2 mRNA, can achieve a significant increase in the level of full-length SMN2 mRNA and full-length SMN protein. This combination strategy of treatment can provide enhanced therapeutic benefit compared to monotherapy and can thus maximize treatment outcomes, e.g., for SMA patients.

In certain aspects, there is provided a pharmaceutical composition for treating or delaying the onset or progression of an SMN-deficiency-related condition, such as SMA, in an individual, the composition comprising a combination of (a) one or more agents that increase the expression of an SMN2 gene or protein, and (b) one or more modulators of SMN2 mRNA splicing or stability that increase the production of functional SMN2 mRNA.

In certain embodiments, the agents that increase the expression of SMN2 gene or protein may include any suitable agent having such activity, including macromolecules and small molecules. Examples of macromolecules are proteins, protein complexes, and glycoproteins, nucleic acids, e.g., DNA, RNA and PNA (peptide nucleic acid). Examples of small molecules are peptides, peptidomimetics (e.g., peptoids), amino acids, amino acid analogs, polynucleotides, polynucleotide analogs, nucleotides, nucleotide analogs, organic or inorganic compounds e.g., heterorganic or organometallic compounds. Any of these agents may be used in combination either consecutively, or concurrently by the methods described herein.

In certain embodiments, the agent that increases the expression of the SMN2 gene or protein is at least one saRNA (referred to as an “SMN2 saRNA” herein) or a recombinant vector thereof, or at least one small molecule compound. In particular embodiments, the agent that increases the expression of the SMN2 gene or protein is at least one SMN2 saRNA. In certain embodiments, the SMN2 saRNA comprises a sense strand and an antisense strand, or comprises a single strand, or mixtures thereof.

In certain embodiments of the present disclosure, the SMN2 mRNA modulator is an antisense oligonucleotide (ASO) or a small molecule, such as a pyridazine derivative. In certain embodiments of the present disclosure, the SMN2 mRNA modulator is selected from Nusinersen (Spinraza®, also referred to as ASO-10-27 herein), Risdiplam, Rigosertib and Branaplam.

In certain embodiments of the present disclosure, the SMN2 saRNA comprises a first strand that is at least 90% identical to (a) the region of the SMN2 gene promoter from −1639 to −1481 (SEQ ID no: 472), (b) the region of the SMN2 gene promoter from −1090 to −1008 (SEQ ID no. 473), (c) the region of the SMN2 gene promoter from −994 to −180 regions (SEQ ID NO: 474), or (d) the region of the SMN2 gene promoter from −144 to −37 (SEQ ID NO: 475).

In certain embodiments of the present disclosure, a first strand of the SMN2 saRNA has at least 75% homology or complementarity with a fragment of the promoter region of the SMN2 gene that is 16-35 nucleotides in length.

In certain embodiments of the present disclosure, a first strand of the SMN2 saRNA molecule has at least 75% homology or complementarity with any nucleotide sequence selected from the group consisting of SEQ ID NOs: 315-471.

In certain embodiments of the present disclosure, the sense strand has at least 75% homology to any of the nucleotide sequences selected from the group consisting of SEQ ID NO: 1-157, and the antisense strand has at least 75% homology to any of the nucleotide sequences selected from the group consisting of SEQ ID NOs: 158-314.

In certain embodiments of the present disclosure, the sense strand comprises a nucleotide sequence selected from any one of SEQ ID NOs: 1-157, and wherein the antisense strand comprises a nucleotide sequence selected from any one of SEQ ID NOs: 158-314.

In certain embodiments of the present disclosure, at least one nucleotide is a chemically modified nucleotide.

In certain embodiments, the composition provided herein further comprises one or more pharmaceutically acceptable carriers, such as an aqueous carrier, liposome, polymeric polymer, or polypeptide.

In certain embodiments of the present disclosure, the composition comprises 1-150 nM of the SMN2 saRNA and 1-50 nM of ASO SMN2 mRNA modulator.

In certain embodiments of the present disclosure, the composition comprises 1-150 nM of the SMN2 saRNA and 1-3000 nM of small molecule pyridazine derivative SMN2 mRNA modulator, such as Risdiplam. In certain embodiments, the composition comprises 300-2000 nM Risdiplam, composition increases the amount of full-length SMN protein in a treated cell by at least 10% compared to a baseline measurement taken prior to treatment or compared to an untreated cell population. In a further embodiment, the composition of the present disclosure decreases the amount of SMN2 Δ7 in a treated cell compared to a baseline measurement taken prior to treatment.

In certain embodiments of the present disclosure, the SMN2 saRNA is DS06-0004 (also known as RAG6-281), DS06-0031 (also known as RAG6-1266) or DS06-0067 (also known as RAG6-293).

Certain embodiments of the present disclosure relate to a method for treating or delaying the onset or progression of the SMN-deficiency-related condition in an individual, the method comprising administering to the individual an effective amount to the pharmaceutical composition comprising (a) one or more agents that increase the expression of SMN2 gene or protein, and (b) one or more modulators of SMN2 mRNA splicing or stability that increase the production of functional SMN2 mRNA. In certain embodiments of the method provided herein, the agent that increases the expression of SMN2 gene or protein is an saRNA (referred to as an “SMN2 saRNA” herein) or a recombinant vector thereof, or is a small molecule compound. In certain embodiments of the method provided herein, the SMN2 mRNA modulator is an antisense oligonucleotide (ASO) or a small molecule compound, such as a pyridazine derivative. In certain embodiments of the present disclosure, the SMN2 mRNA modulator is selected from Nusinersen (Spinraza®, also referred to as ASO-10-27 herein), Risdiplam, Rigosertib and Branaplam.

In certain embodiments of the method provided herein, the SMN2 saRNA comprises one strand that is at least 90% identical to a region of the SMN2 gene promoter from −1639 to −1481 (SEQ ID no: 472), a region of the SMN2 gene promoter from −1090 to −1008 (SEQ ID no: 473), a region of the SMN2 gene promoter from −994 to −180 regions (SEQ ID NO: 474), or a region of the SMN2 gene promoter from −144 to −37 (SEQ ID NO: 475).

In certain embodiments of the method provided herein, one strand of the SMN2 saRNA has at least 75% homology or complementarity with a fragment of the promoter region of the SMN2 gene that is 16-35 nucleotides in length.

In any of the embodiments described herein, the individual has the condition of SMA. In further embodiments, the individual with SMA has decreased or abnormal SMN full length protein expression.

### 5. BRIEF DESCRIPTION OF THE DRAWINGS

FIG. 1 shows the SMN2 gene structure, saRNA target location, and PCR primer location. FIG. 1A shows the SMN2 gene structure and its 2 kb promoter region. The target sites of saRNA DS06-0004, DS06-0067 and DS06-0031 are indicated at the locations of −281, −293 and −1266, respectively, relative to the transcription start site (TSS) of SMN2. FIG. 1B shows the location of PCR primers used for RT-qPCR (SMN2FL F+SMN2FL R, SMNΔ7 F+SMNΔ7 R) and semi-quantitative RT-PCR (SMN-exon6-F+SMN-exon8-R).

FIG. 2 shows the differences between SMN1 and SMN2 genes and a schematic of semi-quantitative RT-PCR/DdeI digestion assay. A G→A variant in exon 8 of SMN2 creates a recognition site for DdeI restriction enzyme (A). The PCR primer pair SMN-exon6-F and SMN-exon8-R amplifies a 507 bp products (SMN2FL) and a 453 bp product (SMN2 Δ7). To differentiate SMN1 and SMN2 products, digestion with DdeI cuts SMN2FL products into 392 bp and 115 bp fragments (B), and SMN2 Δ7 products into 338 bp and 115 bp fragments (C).

FIG. 3A-3G shows the effect of saRNA (DS06-0004), ASO-10-27 and Risdiplam on the expression of full-length (SMN2FL) and exon 7 skipped (SMN2 Δ7) SMN2 mRNA in GM03813 cells. GM03813 cells were treated with the indicated concentration of ASO-10-27, saRNA (DS06-0004) and Risdiplam for 72 hours. Mock samples, as a control treatment, were transfected in the absence of an oligonucleotide. mRNA levels of SMN2FL and SMNΔ7 were determined by RT-qPCR using two pairs of primers in separate PCR reactions. FIG. 3A-3C show the mRNA level of SMN2FL and SMNΔ7 determined by RT-qPCR. FIG. 3D shows the mRNA level of SMN2FL and SMNΔ7 determined by semi-quantitative PCR. The PCR products of SMN2 were digested by DdeI enzyme and separated on a 2% agarose gel. TBP gene was also amplified as a control for RNA loading. FIG. 3E-3G show SMN2FL and SMN2 Δ7 levels derived from quantifying PCR product band intensity in FIG. 3D. The values (y-axis) are SMN2 band intensity relative to Mock treatment after normalizing to the band intensity of TBP. SMN2FL, SMN2 full-length PCR product after digestion (392 bp); SMNΔ7, SMN exon 7 skipped PCR product after digestion (338 bp); Ladder: 100 bp DNA marker.

FIG. 4A-4E shows the combinatory effect of saRNA (DS06-0004) and ASO-10-27 on the expression of SMN2FL and SMN2 Δ7 SMN2 mRNA in GM00232 cells. ASO-10-27 and DS06-0004 were transfected individually or in combination into GM00232 cells at the indicated concentrations for 72 hours. Mock samples, as a control treatment, were transfected in the absence of oligonucleotides. Total cellular RNA was extracted using Qiagen RNeasy kits from the treated cells for reverse transcription to obtain cDNA, and then the mRNA level of SMN2FL and SMNΔ7 was determined by RT-qPCR. FIGS. 4A and 4D show mRNA level of SMN2FL and SMNΔ7 determined by RT-qPCR. FIG. 4B shows the mRNA level of SMN2FL and SMNΔ7 determined by semi-quantitative PCR. The PCR products of SMN2 were digested by DdeI enzyme and separated on a 2% agarose gel. TBP gene was also amplified as a control for RNA loading. FIGS. 4C and 4E show SMN2FL and SMN2 Δ7 levels derived from quantifying the PCR product band intensity in FIG. 4B. The values (y-axis) are band intensity of SMN2FL and SMN2 Δ7 relative to Mock treatment after normalizing to that of TBP. SMN2FL, SMN2 full-length PCR product after digestion (392 bp); SMNΔ7, SMN exon 7 skipped PCR product after digestion (338 bp), ASO, ASO-10-27.

FIG. 5A-5C shows the combinatory effect of saRNA (DS06-0004) and ASO-10-27 on expression of full-length SMN protein in type I SMA cells GM00232. ASO-10-27 and DS06-0004 were transfected individually or in combination into GM00232 cells at the indicated concentrations for 72 hours. Mock samples, as a control treatment, were transfected in the absence of oligonucleotides. Proteins were harvested from the treated cells and immunoblotted by western blotting assay using an antibody against human SMN protein. An antibody against α/β-Tubulin was also blotted to serve as a control for protein loading. FIG. 5A shows SMN protein expression from cells Mock treated and treated with ASO-10-27 alone or its combination with DS06-0004. FIG. 5B shows SMN protein expression from cells Mock treated and treated with DS06-0004 alone or its combination with ASO-10-27. FIG. 5C shows relative fold changes of SMN protein levels derived from quantifying the band intensity of FIGS. 5A and 5B. Values (y-axis) are relative band intensity of SMN protein after being normalized to that of α/β-Tubulin. ASO, ASO-10-27.

FIG. 6A-6E shows the combinatory effect of saRNA (DS06-0004) and ASO-10-27 on the expression of SMN2FL and SMN2 Δ7 SMN2 mRNA in GM03813 cells. ASO-10-27 and DS06-0004 were transfected individually or in combination into GM03813 cells at the indicated concentrations for 72 hours. Mock samples, as a control treatment, were transfected in the absence of oligonucleotides. Total cellular RNA was extracted using Qiagen RNeasy kits from the treated cells for reverse transcription to obtain cDNA, and then the mRNA level of SMN2FL and SMNΔ7 was determined by RT-qPCR. FIGS. 6A and 6D show mRNA level of SMN2FL and SMNΔ7 determined by RT-qPCR. FIG. 6B shows the mRNA level of SMN2FL and SMNΔ7 determined by semi-quantitative PCR. The PCR products of SMN2 were digested by DdeI enzyme and separated on a 2% agarose gel. TBP gene was also amplified as a control for RNA loading. FIGS. 6C and 6E show SMN2FL and SMN2 Δ7 levels derived from quantifying the PCR product band intensity in FIG. 6B. The values (y-axis) are band intensity of SMN2FL and SMN2 Δ7 relative to Mock treatment after normalizing to that of TBP. SMN2FL, SMN2 full-length PCR product after digestion (392 bp); SMNΔ7, SMN exon 7 skipped PCR product after digestion (338 bp), ASO, ASO-10-27.

FIG. 7A-7C shows the combinatory effect of saRNA (DS06-0004) and ASO-10-27 on expression of full-length SMN protein in type II SMA cells GM03813. ASO-10-27 and DS06-0004 were transfected individually or in combination into GM00232 cells at the indicated concentrations for 72 hours. Mock samples, as a control treatment, were transfected in the absence of oligonucleotides. Proteins were harvested from the treated cells and immunoblotted by western blotting assay using an antibody against human SMN protein. An antibody against α/β-Tubulin was also blotted to serve as a control for protein loading. FIG. 7A shows SMN protein expression from cells Mock treated and treated with ASO-10-27 alone or its combination with DS06-0004. FIG. 7B shows SMN protein expression from cells mock treated and treated with DS06-0004 alone or its combination with ASO-10-27. FIG. 7C shows relative fold changes of SMN protein levels derived from quantifying the band intensity of FIGS. 7A and 7B. Values (y-axis) are relative band intensity of SMN protein after being normalized to that of a/0-Tubulin. ASO, ASO-10-27.

FIG. 8A-8F shows the combinatory effect of saRNA (DS06-0004) and Risdiplam on the expression of SMN2FL and SMN2 Δ7 SMN2 mRNA in GM00232 cells. Risdiplam and DS06-0004 were transfected individually or in combination into GM00232 cells at the indicated concentrations for 72 hours. DMSO samples serve as a vehicle control for Risdiplam and Mock treatment as a control for saRNA transfection was transfected in the absence of oligonucleotides. Total cellular RNA was extracted using Qiagen RNeasy kits from the treated cells for reverse transcription to obtain cDNA, and then the mRNA level of SMN2FL and SMNΔ7 was determined by RT-qPCR. FIG. 8A shows mRNA level of SMN2FL and SMNΔ7 determined by RT-qPCR. FIGS. 8B and 8C show relative SMN2FL mRNA levels in cells combo-treated with different concentrations of DS06-0004 and Risdiplam. FIG. 8D shows the mRNA level of SMN2FL and SMNΔ7 determined by semi-quantitative PCR. The PCR products of SMN2 were digested by DdeI enzyme and separated on a 2% agarose gel. TBP gene was also amplified as a control for RNA loading. FIGS. 8E and 8F show SMN2FL levels derived from quantifying PCR product band intensity in FIG. 8D. The values (y-axis) are the band intensity of SMN2FL and SMN2 Δ7 relative to Mock or DMSO treatment after normalizing to that of TBP. SMN2FL, SMN2 full-length PCR product after digestion (392 bp); SMNΔ7, SMN exon 7 skipped PCR product after digestion (338 bp).

FIG. 9A-9B shows the combinatory effect of saRNA (DS06-0004) and Risdiplam on expression of full-length SMN protein in type I SMA cells GM00232. Risdiplam and DS06-0004 were transfected individually or in combination into GM00232 cells at the indicated concentrations for 72 hours. DMSO samples serve as a vehicle control for Risdiplam and Mock treatment as a control for saRNA transfection was transfected in the absence of oligonucleotides. Proteins were harvested from the treated cells and immunoblotted by western blotting assay using an antibody against human SMN protein. An antibody against α/β-Tubulin was also blotted to serve as a control for protein loading. FIG. 9A shows SMN protein expression from cells DMSO treated and treated with Risdiplam alone or its combination with DS06-0004. FIG. 9B shows relative fold changes of SMN protein levels derived from quantifying the band intensity of FIG. 9A. Values (y-axis) are relative band intensity of SMN protein after being normalized to that of a/p-Tubulin.

FIG. 10A-10F shows the combinatory effect of saRNA (DS06-0004) and Risdiplam on the expression of SMN2FL and SMN2 Δ7 SMN2 mRNA in GM03813 cells. Risdiplam and DS06-0004 were transfected individually or in combination into GM03813 cells at the indicated concentrations for 72 hours. DMSO samples serve as a vehicle control for Risdiplam and Mock treatment as a control for saRNA transfection was transfected in the absence of oligonucleotides. Total cellular RNA was extracted using Qiagen RNeasy kits from the treated cells for reverse transcription to obtain cDNA, and then the mRNA level of SMN2FL and SMNΔ7 was determined by RT-qPCR. FIG. 10A shows mRNA level of SMN2FL and SMNΔ7 determined by RT-qPCR. FIGS. 10B and 6C show relative SMN2FL mRNA levels in cells combo-treated with different concentrations of DS06-0004 and Risdiplam. FIG. 10D shows the mRNA level of SMN2FL and SMNΔ7 determined by semi-quantitative PCR. The PCR products of SMN2 were digested by DdeI enzyme and separated on a 2% agarose gel. TBP gene was also amplified as a control for RNA loading. FIGS. 10E and 10F show SMN2FL levels derived from quantifying PCR product band intensity in FIG. 10D. The values (y-axis) are the band intensity of SMN2FL and SMN2 Δ7 relative to Mock or DMSO treatment after normalizing to that of TBP. SMN2FL, SMN2 full-length PCR product after digestion (392 bp); SMNΔ7, SMN exon 7 skipped PCR product after digestion (338 bp).

FIG. 11A-11B shows the combinatory effect of saRNA (DS06-0004) and Risdiplam on the expression of full-length SMN protein in type II SMA cell GM03813. Risdiplam and DS06-0004 were transfected individually or in combination into GM03813 cells at the indicated concentrations for 72 hours. DMSO samples serve as a vehicle control for Risdiplam and Mock treatment as a control for saRNA transfection was transfected in the absence of oligonucleotides. Proteins were harvested from the treated cells and immunoblotted by western blotting assay using an antibody against human SMN protein. An antibody against α/β-Tubulin was also blotted to serve as a control for protein loading. FIG. 11A shows SMN protein expression from cells mock treated and treated with Risdiplam alone or its combination with DS06-0004. FIG. 11B shows relative fold changes of SMN protein levels derived from quantifying the band intensity of FIG. 11A. Values (y-axis) are relative band intensity of SMN protein after being normalized to that of a/p-Tubulin.

FIG. 12A-12E shows the combinatory effect of saRNAs (DS06-0031 and DS06-0067) and ASO-10-27 on the expression of SMN2FL and SMN2 Δ7 SMN2 mRNA and SMN protein in GM03813 cells. DS06-0031 or DS06-0067 was transfected individually or in combination with ASO-10-27 into GM03813 cells at 10 nM for 72 hours. Mock samples, as a control treatment, were transfected in the absence of oligonucleotides. dsCon2 was transfected as an unrelated oligonucleotide control. DS06-332i is an siRNA for SMN2 and was transfected as a control treatment. Total cellular RNA was extracted using Qiagen RNeasy kits from the treated cells for reverse transcription to obtain cDNA, and then the mRNA level of SMN2FL and SMNΔ7 was determined by RT-qPCR and semi-quantitative RT-PCR. Proteins were harvested from the treated cells and immunoblotted by western blotting assay using an antibody against human SMN protein. An antibody against α/β-Tubulin was also blotted to serve as a control for protein loading. FIG. 12A shows mRNA level of SMN2FL and SMNΔ7 determined by RT-qPCR. FIG. 12B shows the mRNA level of SMN2FL and SMNΔ7 determined by semi-quantitative PCR and separated on a 2% agarose gel. TBP gene was also amplified as a control for RNA loading. FIG. 12C shows SMN2FL and SMN2 Δ7 mRNA levels derived from quantifying the PCR product band intensity in FIG. 12B. The values (y-axis) are band intensity of SMN2FL and SMN2 Δ7 relative to Mock treatment after normalizing to that of TBP. SMN2FL, SMN2 full-length PCR product (507 bp): SMNΔ7, SMN exon 7 skipped PCR product (453 bp). FIG. 12D shows a western blot of SMN protein expression. FIG. 12E shows relative fold changes of SMN protein levels derived from quantifying the band intensity of FIG. 12D. Values (y-axis) are relative band intensity of SMN protein after being normalized to that of α/β-Tubulin.

FIG. 13A-13C show the combinatory effect of saRNA (LNP-R6-04M1) and LNP-ASO-10-27 or Risdiplam on the expression of full-length (SMN2FL) and exon 7 skipped (SMN2 Δ7) SMN2 mRNA in SMA type III mice (PND7). Three groups of SMA type III mice were treated with Treatment Group 1 (n=3)—administered with LNP-R6-04M1 alone on postnatal day 1 (P1, 10 μg) and postnatal day 3 (P3, 10 μg), Treatment Group 2 (n=3)—administered with LNP-R6-04M1 in combination with LNP-ASO-10-27 (P1, 10 μg and P3, 10 μg), and Treatment Group 3 (n=3)—administered with LNP-R6-04M1 and Risdiplam (0.3 mg/kg, 1 mg/kg and 3 mg/kg) by lateral ventricle injection (ICV) at the indicated concentrations. Mice that were treated with saline served as a non-treatment control. Following treatment, RNA was isolated from two tissues (brain and liver) using Qiagen RNeasy kits for reverse transcription to obtain cDNA, and then the mRNA level of SMN2FL and SMNΔ7 was determined by RT-qPCR. FIG. 13A shows the mRNA level of SMN2FL and SMNΔ7 determined by RT-qPCR in brain of SMA type II mice. FIG. 13B shows SMN2FL mRNA level of SMN2FL and SMNΔ7 determined by RT-qPCR in liver of SMA type III mice. FIG. 13C shows SMN2FL mRNA level of SMN2FL and SMNΔ7 determined by RT-qPCR in spinal cord of SMA type III mice. SMN2FL and SMN2 Δ7 mRNA levels are shown as mean values of three animals/group (n=3-7) relative to saline treatment after normalizing to Tbp reference levels.

### 6. DETAILED DESCRIPTION OF THE INVENTION

The present invention is based on investigations related to methods that activate/upregulate SMN2 gene expression and increase the amount of expression of full-length SMN2 in order to improve therapeutic effects for SMN-deficiency-related conditions.

In the present disclosure, we further show that combinatory treatment of SMA patient cells with an SMN2 saRNA and an SMN2 mRNA modulator, e.g., an ASO, such as Nusinersen, or a small pyridazine derivative including but not limited to Risdiplam and Branaplam, can achieve significantly higher levels of full-length SMN2 mRNA and SMN protein than the amount that can be achieved by either of the compounds used alone. This combination strategy for treatment can provide enhanced therapeutic benefit compared to monotherapy, for example, by improvements in the clinical symptoms of a patient diagnosed with an SMN-deficiency-related condition, or by reducing unwanted side effects in connection with monotherapy, and thus maximizing the treatment outcome of patients, such as SMA patients.

Unless otherwise defined, all the technological and scientific terms used therein have the same meanings as those generally understood by those of ordinary skill in the art covering the present invention.

In the present application, singular forms, such as “a” and “this”, include plural objects, unless otherwise specified clearly in the context.

### 6.1. Definitions

As used herein, the term “SMN-deficiency-related conditions” refers to a disease caused by deficiency in SMN full-length protein due to any cause. “SMN-deficiency-related conditions” include, but are but are not limited to, spinal muscular atrophy (SMA), neurogenic-type arthrogryposis multiplex congenita (congenital AMC), and amyotrophic lateral sclerosis (ALS). For SMN1 (human), the GenBank gene reference is Gene ID: 6606.

The terms “spinal muscular atrophy” or “SMA” include, but are not limited to, spinal muscular atrophy (SMA) types 1 through 4; proximal spinal muscular atrophy; childhood-onset SMA Type I (Werdnig-Hoffmann disease); Type II (intermediate, chronic form), Type III (Kugelberg-Welander disease, or Juvenile Spinal Muscular Atrophy), and a relatively recently categorized adult-onset form Type IV. Meeting report: International SMA Consortium meeting. Neuromuscul Disord.; 2: 423-428. The term SMA also includes late-onset SMA (also known as SMA types 3 and 4, mild SMA, adult-onset SMA and Kugelberg-Welander disease). The term SMA also includes other forms of SMA, including X-linked disease, spinal muscular atrophy with respiratory distress (SMARD), spinal and bulbar muscular atrophy (Kennedy's disease, or Bulbo-Spinal Muscular Atrophy), and distal spinal muscular atrophy. The term SMA includes all forms of SMA described in Arnold, W. D., Kassar, D. & Kissel, J. T. Spinal muscular atrophy: Diagnosis and management in a new therapeutic era. Muscle and Nerve (2015); Butchbach, M. E. R. Copy Number Variations in the Survival Motor Neuron Genes: Implications for Spinal Muscular Atrophy and Other Neurodegenerative Diseases. Front. Mol. Biosci. (2016).

When SMA symptoms are present at birth or by the age of 6 months, the disease is called SMA type 1 (also called infantile onset or Werdnig-Hoffmann disease). Babies typically have generalized muscle weakness, a weak cry, and breathing distress. They often have difficulty swallowing and sucking, and don't reach the developmental milestone of being able to sit up unassisted. These babies have increased risk of aspiration and failure to thrive. Typically, these babies have two or three copies of the SMN2 gene. (Butchbach, M. E. R. Copy Number Variations in the Survival Motor Neuron Genes: Implications for Spinal Muscular Atrophy and Other Neurodegenerative Diseases. Front. Mol. Biosci. (2016) which is incorporated herein in its entirety)

When SMA has its onset between the ages of 3 and 15 months and before the child can stand or walk independently, it is called SMA type 2, or intermediate SMA or Dubowitz disease. Children with SMA type 2 generally have three copies of the SMN2 gene (Arnold, W. D., Kassar, D. & Kissel, J. T. Spinal muscular atrophy: Diagnosis and management in a new therapeutic era. Muscle and Nerve (2015) which is incorporated herein in its entirety). Muscle weakness is predominantly proximal (close to the center of the body) and involves the lower limbs more than the upper limbs. Usually, the face and the eye muscles are unaffected. (Butchbach, M. E. R. Copy Number Variations in the Survival Motor Neuron Genes: Implications for Spinal Muscular Atrophy and Other Neurodegenerative Diseases. Front. Mol. Biosci. (2016) which is incorporated herein in its entirety).

Late-onset SMA (also known as SMA types 3 and 4, mild SMA, adult-onset SMA and Kugelberg-Welander disease) results in variable levels of weakness. Patients with type 3 SMA have 3 to 4 copies of the SMN2 gene. SMA type 3 (juvenile onset) accounts for 30% of overall SMA cases (Arnold, W. D., Kassar, D. & Kissel, J. T. Spinal muscular atrophy: Diagnosis and management in a new therapeutic era. Muscle and Nerve (2015)). Symptoms usually appear between age 18 months and adulthood. Affected individuals achieve independent mobility. However, proximal weakness in these patients might cause falls and difficulty with climbing stairs. Over time, many lose their ability to stand and walk, so instead use a wheelchair to move around. Most of these patients develop foot deformities, scoliosis, and respiratory muscle weakness.

SMA type 4 is late-onset and accounts for less than 5% of overall SMA cases. These patients have four to eight copies of the SMN2 gene (Butchbach, M. E. R. Copy Number Variations in the Survival Motor Neuron Genes: Implications for Spinal Muscular Atrophy and Other Neurodegenerative Diseases. Front. Mol. Biosci. (2016)). Age of onset is not defined but is usually after age 30. Type 4 is a mild form of SMA and therefore lifespan remains normal. Patients can achieve motor milestones and maintain their mobility throughout life.

As used herein, the terms “subject” and “individual” are used interchangeably herein to mean any living organism that may be treated with compounds of the present disclosure. The term “patient” means a human subject or individual, including disclosure infants, children and adults.

A “therapeutically effective amount” of a composition is an amount sufficient to achieve a desired therapeutic effect, and therefore does not require cure or complete remission. In embodiments of the present disclosure, therapeutic efficacy is an improvement in any of the disease indicators, and a therapeutically effective amount is sufficient to cause an improvement in a clinically significant condition/symptom in the treated individual. The phrases “therapeutically effective amount” and “effective amount” are used herein to mean an amount sufficient to reduce by at least about 15 percent, preferably by at least 50 percent, more preferably by at least 90 percent, and most preferably prevent, a clinically significant deficit in the activity, function and response of the individual being treated.

The effective amount can vary depending on such factors as the size and weight of the subject, the type of illness, or the particular compounds of the invention. For example, the choice of the compound of the invention can affect what constitutes an “effective amount.” One of ordinary skill in the art would be able to study the factors contained herein and make the determination regarding the effective amount of the compounds of the invention without undue experimentation.

The regimen of administration can affect what constitutes an effective amount. The compound of the invention can be administered to the subject either prior to or after the onset of an SMN-deficiency-related condition. Further, several divided dosages, as well as staggered dosages, can be administered daily or sequentially, or the dose can be continuously infused, or can be a bolus injection. Further, the dosages of the compound(s) of the invention can be proportionally increased or decreased as indicated by the exigencies of the therapeutic or prophylactic situation. The dosage at which compositions of the present application can be administered can vary within wide limits and will, of course, be fitted to the individual requirements in each case.

The terms “treat,” “treated,” “treating”, or “treatment” as used herein have the meanings commonly understood in the medical arts, and therefore do not require cure or complete remission, and include any beneficial or desired clinical results. Nonlimiting examples of such beneficial or desired clinical results are prolonging survival as compared to expected survival without treatment, reduced symptoms including one or more of the followings: weakness and atrophy of proximal skeletal muscles, inability to sit or walk independently, difficulties in swallowing, breathing, etc.

As used herein, “preventing” or “delaying” a disease refers to inhibiting the full development of a disease.

The term “biological sample” refers to any tissue, cell, fluid, or other material derived from an organism (e.g., human subject). In certain embodiments, the biological sample is serum or blood.

As used herein, the term “sequence identity” or “sequence homology” means that one oligonucleotide strand (sense or antisense) of a saRNA has at least 80% similarity with a region on the coding strand or template strand of the promoter sequence of a target gene.

In embodiments of the present disclosure, the target gene is SMN2. By “target sequence” is meant a sequence fragment to which the sense oligonucleotide strand or antisense oligonucleotide of an SMN2 saRNA is homologous or complementary in the promoter sequence of the target gene. “Target gene promoter sequence” refers to a non-coding sequence of a target gene, and in the context of the present disclosure “complementary to the promoter sequence of the target gene” refers to the coding strand of the sequence, also referred to as the non-template strand, i.e., a nucleic acid sequence that is the same sequence as the coding sequence of the gene.

As used herein, the terms “sense strand” and “sense oligonucleotide strand” are interchangeable, and the sense oligonucleotide strand of a small activating ribonucleic acid (saRNA) molecule refers to a first nucleic acid strand comprising a coding strand of a promoter sequence of a target gene in a duplex of saRNA.

As used herein, the terms “antisense strand” and “antisense oligonucleotide strand” are interchangeable, and the antisense oligonucleotide strand of an saRNA molecule refers to a second nucleic acid strand in a duplex of saRNA that is complementary to the sense oligonucleotide strand.

As used herein, the term “first oligonucleotide strand” can be a sense strand or an antisense strand. The sense strand of a saRNA refers to an oligonucleotide strand having homology with the coding strand of the promoter DNA sequence of the target gene in the saRNA duplex. The antisense strand refers to an oligonucleotide strand complementary with the sense strand in the saRNA duplex.

As used herein, the term “second oligonucleotide strand” can also be a sense strand or an antisense strand. If the first oligonucleotide strand is a sense strand, the second oligonucleotide strand is an antisense strand; and if the first oligonucleotide strand is an antisense strand, the second oligonucleotide strand is a sense strand.

The term “promoter” as used herein refers to a nucleic acid sequence, which encodes no proteins and plays a regulatory role for the transcription of a protein-coding or RNA-coding nucleic acid sequence by associating with them spatially. Generally, a eukaryotic promoter contains 100 to 5,000 base pairs, although this length range is not intended to limit the term of “promoter” as used herein. Although the promoter sequence is generally located at the 5′ terminus of a protein-coding or RNA-coding sequence, it also exists in exon and intron sequences.

As used herein, the term “coding strand” refers to the DNA strand in the target gene that cannot be transcribed, the nucleotide sequence of which is identical to the sequence of the RNA produced by transcription (in RNA the T in DNA is replaced by U). The coding strand of the double-stranded DNA sequence of the target gene promoter described in the present disclosure refers to the promoter sequence on the same DNA strand as the DNA coding strand of the target gene.

As used herein, the term “template strand” refers to another strand of double-stranded DNA of a target gene that is complementary to the coding strand and that can be transcribed as a template into RNA that is complementary to the transcribed RNA base (A-U, G-C). During transcription, RNA polymerase binds to the template strand and moves along the 3→5′ direction of the template strand, catalyzing RNA synthesis in the 5′→3′ direction. The template strand of the double-stranded DNA sequence of the target gene promoter described in the present disclosure refers to the promoter sequence on the same DNA strand as the DNA template strand of the target gene.

As used herein, the term “transcription start site” or TSS refers to a nucleotide that marks the initiation of transcription on the template strand of a gene. The transcription start site may be present on the template strand of the promoter region. A gene may have more than one transcription start site.

As used herein, the term “overhang” refers to an oligonucleotide strand end (5′ or 3′) with non-base paired nucleotide(s) resulting from another strand extending beyond one of the strands within the double stranded oligonucleotide. Single stranded regions extending beyond the 3′ and/or 5′ ends of the duplexes are referred to as overhangs. In certain embodiments, the overhang is from 0 to 6 nucleotides in length. It is understood that an overhang of 0 nucleotides means that there is no overhang.

As used herein, the terms “gene activation”, “activating gene expression”, “gene upregulation” and “upregulating gene expression” can be used interchangeably, and means an increase or upregulation in transcription, translation, expression or activity of a certain nucleic acid sequence as determined by measuring the transcription level, mRNA level, protein level, enzymatic activity, methylation state, chromatin state or configuration, translation level or the activity or state in a cell or biological system of a gene. These activities or states can be determined directly or indirectly. In addition, “gene activation” or “activating gene expression” refers to an increase in activity associated with a nucleic acid sequence, regardless the mechanism of such activation. For example, gene activation occurs at the transcriptional level to increase transcription into RNA and the RNA is translated into a protein, thereby increasing the expression of the protein.

As used herein, the terms “small activating RNA”, “saRNA” and “small activating ribonucleic acid” can be used interchangeably and refer to a ribonucleic acid molecule that can upregulate target gene expression. It can be a double-stranded nucleic acid molecule composed of a first nucleic acid strand containing a ribonucleotide sequence with sequence homology with the non-coding nucleic acid sequence (such as a promoter and an enhancer) of a target gene and a second nucleic acid strand containing a nucleotide sequence complementary with the first strand. The saRNA can also be comprised of a synthesized or vector-expressed single-stranded RNA molecule that can form a hairpin structure by two complementary regions within the molecule, wherein the first region contains a ribonucleotide sequence having sequence homology with the target sequence of a promoter of a gene, and a ribonucleotide sequence contained in the second region is complementary with the first region. The length of the duplex region of the saRNA molecule is typically about 10 to about 50, about 12 to about 48, about 14 to about 46, about 16 to about 44, about 18 to about 42, about 20 to about 40, about 22 to about 38, about 24 to about 36, about 26 to about 34, and about 28 to about 32 base pairs, and typically about 10, about 15, about 20, about 25, about 30, about 35, about 40, about 45 or about 50 base pairs. In addition, the terms “small activating RNA”, “saRNA” and “small activating ribonucleic acid” also contain nucleic acids other than the ribonucleotide, including, but not limited to, modified nucleotides or analogues.

As used herein, the term “synthetic” refers to the manner in which oligonucleotides are synthesized, including any means capable of synthesizing or chemically modifying RNA, such as chemical synthesis, in vitro transcription, vector expression, and the like.

### 6.2. Compositions of the Combination of saRNAs and mRNA Modulators

Certain embodiments of the present disclosure provide a composition comprising a combination of (a) one or more agents that increase the expression of the SMN2 gene or proteins, and (b) one or more modulators of SMN2 mRNA splicing or stability that increase the production of functional SMN2 mRNA.

Administration of this combination to a patient treats or delays the onset of an SMN-deficiency-related condition, such as spinal muscular atrophy. In certain embodiments, the described combination increases the amount of a full-length SMN protein by, for example, activating/up-regulating SMN2 transcription in conjunction with modulating splicing for exon 7 inclusion to increase the amount of full-length SMN2 mRNA. In certain embodiments, full-length SMN protein is increased in an amount sufficient to reduce the symptoms associated with an SMN-deficiency-related condition. In certain embodiments, full-length SMN protein is increased by at least 10%.

6.2.1. Agents that Increase the Expression of the SMN2 Gene or Protein

In certain embodiments, at least one of the one or more agents that increase the expression of the SMN2 gene or protein is an saRNA. The SMN2 saRNA activates or upregulates the expression of an SMN2 gene in a cell in which the SMN2 gene is normally expressed.

In typical embodiments, a first strand of the SMN2 saRNA comprises a segment that has at least 75% sequence identity or sequence complementarity to a 16-35 nucleotide fragment of the promoter region of the SMN2 gene thereby effecting activation or upregulation of expression of the gene.

In particular, the first strand of the SMN2 saRNA has homology or complementarity with a region of the SMN2 gene promoter from a region of the SMN2 gene promoter from −1639 to −1481 (SEQ ID no: 472), a region of the SMN2 gene promoter from −1090 to −1008 (SEQ ID no: 473), a region of the SMN2 gene promoter from −994 to −180 regions (SEQ ID NO: 474), or a region of the SMN2 gene promoter from −144 to −37 (SEQ ID NO: 475), and have a homology or complementarity of at least 75%, such as at least about 79%, about 80%, about 85%, about 900, about 95%, or about 99%. More specifically, one strand of the SMN2 saRNA has at least 75%, e.g., at least about 79%, or about 99% homology or complementarity with any nucleotide sequence selected from the group consisting of SEQ ID NO: 315-471.

In the present disclosure, the SMN2 saRNA comprises a sense nucleic acid fragment and an antisense nucleic acid fragment. The sense nucleic acid fragment and the antisense nucleic acid fragment comprise complementary regions capable of forming a double-stranded nucleic acid structure that facilitates expression of the SMN2 gene in a cell by the RNA activation mechanism. Sense nucleic acid fragments and antisense nucleic acid fragments of saRNAs may be present on two different nucleic acid strands or may be present on the same nucleic acid strand. When the sense and antisense nucleic acid fragments are present on two strands at least one strand of the saRNA has a 3′ overhang of 0-6 nucleotides in length, preferably both strands have a 3′ overhang of 2 or 3 nucleotides in length, and preferably the nucleotides of the overhang are deoxythymine (dT). When a sense nucleic acid fragment and an antisense nucleic acid fragment of an saRNA are present on the same nucleic acid strand, preferably the saRNA is a single-stranded hairpin-structured nucleic acid molecule, wherein the complementary regions of the sense nucleic acid fragment and the antisense nucleic acid fragment form a double-stranded nucleic acid structure. In such an saRNA, the sense nucleic acid fragment and antisense nucleic acid fragment are 16-35 nucleotides in length and may be 16, 17, 18, 19, 20, 21, 22, 23, 24, 25, 26, 27, 28, 29, 30, 31, 32, 33, 34, or 35 nucleotides.

In one embodiment, the sense strand of the SMN2 saRNA of the present disclosure has at least 75%, e.g., at least about 79%, about 80%, about 85%, about 90%, about 95%, homology with any nucleotide sequence selected from the group consisting of SEQ ID NO: 1-157. Or about 99% homology, and its antisense strand has at least 75%, or about 99% homology to any nucleotide sequence selected from the group consisting of SEQ ID NO: 158-314. Specifically, the sense strand of the SMN2 saRNA of the present disclosure comprises, or alternatively consists of, any nucleotide sequence selected from the group consisting of SEQ ID NO: 1-157, or any nucleotide sequence selected from the group consisting of SEQ ID NO: 1-157; The antisense strand of the SMN2 saRNA of the disclosure comprises any nucleotide sequence selected from SEQ ID NO: 158-314, or alternatively consists of any nucleotide sequence selected from SEQ ID NO: 158-314, or any nucleotide sequence selected from SEQ ID NO: 158-314.

In certain embodiments of the present disclosure, the SMN2 saRNA comprises a sense nucleic acid strand and an antisense nucleic acid strand, the sense nucleic acid strand comprising at least one region that is complementary to at least one region on the antisense nucleic acid strand to form a double-stranded nucleic acid structure capable of activating expression of the SMN2 gene in a cell.

In certain embodiments of the present disclosure, the sense nucleic acid strand and the antisense nucleic acid strand are located on two different nucleic acid strands.

In certain embodiments of the present disclosure, the sense nucleic acid fragment and the antisense nucleic acid fragment are located on the same nucleic acid strand, forming a hairpin single-stranded nucleic acid molecule, wherein the complementary regions of the sense nucleic acid fragment and the antisense nucleic acid fragment form a double-stranded nucleic acid structure.

In certain embodiments of the present disclosure, at least one of the nucleic acid strands has a 3′overhang of 0 to 6 nucleotides in length.

In certain embodiments of the present disclosure, both of the nucleic acid strands have 3′overhangs of 2-3 nucleotides in length.

In certain embodiments of the present disclosure, the sense and antisense nucleic acid strands are 16 to 35 nucleotides in length, respectively.

All nucleotides of the SMN2 saRNA described herein may be natural, i.e., non-chemically modified, nucleotides or at least one nucleotide may be chemically modified nucleotides, the chemical modification being one or a combination of the following modifications:
(1) Modifications to phosphodiester linkages of nucleotides in the nucleotide sequence of the SMN2 saRNA;
(2) Modification of the 2′-OH of ribose in the nucleotide sequence of the SMN2 saRNA;
(3) Modifications to bases in the nucleotide sequence of the SMN2 saRNA.

Chemical modifications of nucleotides or saRNA the present disclosure are well known to those skilled in the art, and modifications of the phosphodiester bond refer to modifications of oxygen in the phosphodiester bond, including phosphorothioate modifications and boronated phosphate modifications. Both modifications stabilize the SMN2 saRNA structure, maintaining high specificity and high affinity for base pairing.

The ribose modification refers to a modification of the 2′-OH in a nucleotide pentose, i.e., introduction of certain substituents at the hydroxyl position of the ribose, e.g., 2′-fluoro modification, 2′-oxomethyl modification, 2′-oxyethylenemethoxy modification, 2,4′-dinitrophenol modification, locked nucleic acid (LNA), 2′-amino modification, 2′-deoxy modification.

By base modification is meant modification of the base of the nucleotide, e.g., 5′-bromouracil modification, 5′-iodouracil modification, N-methyluracil modification, 2,6-diaminopurine modification.

These modifications may increase the bioavailability of the SMN2SMN2 saRNA, increase affinity for the target sequence, and enhance resistance to nuclease hydrolysis in a cell.

In addition, to facilitate entry of an SMN2 saRNA into a cell, lipophilic groups such as cholesterol may be introduced at the ends of the sense or antisense strands of the SMN2 saRNA on the basis of the above modifications to facilitate action through a cell membrane composed of lipid bilayers and gene promoter regions within the nuclear membrane and nucleus.

The SMN2 saRNA of the present disclosure which, upon contact with a cell, are effective in activating or up-regulating the expression of the SMN2 gene in the cell, preferably by at least 10%.

One aspect of the disclosure provides a cell comprising an SMN2 saRNA of the present disclosure or a nucleic acid encoding an SMN2 saRNA of the present disclosure. In one embodiment, the cell is a mammalian cell, preferably a human cell. Such cells may be ex vino, such as cell lines or cell lines, and the like, or may be present in mammalian bodies, such as humans, including infants, children or adults.

Another aspect of the invention provides a pharmaceutical composition comprising an SMN2 saRNA as described above or a nucleic acid encoding an SMN2 saRNA according to the invention, a SMN2 mRNA modulator and one or more pharmaceutically acceptable carriers. In one embodiment, the pharmaceutically acceptable carrier includes one or more of an aqueous carrier, liposome, polymeric polymer, and polypeptide. In one embodiment, the pharmaceutically acceptable carrier includes one or more of aqueous carriers, liposomes, polymeric polymers, or polypeptides. In one embodiment, the aqueous carrier may be, for example, RNase-free water, or RNase-free buffer. The composition may contain 1-150 nM, for example 1-100 nM, for example 1-50 nM, for example 1-20 nM, for example 10-100 nM, 10-50 nM, 20-50 nM, 20-100 nM, for example 50 nM of the aforementioned SMN2 saRNASMN2 saRNA or nucleic acid encoding the SMN2 saRNA according to the invention.

Another aspect of the present disclosure relates to the use of an SMN2 saRNA as described herein, a nucleic acid encoding an SMN2 saRNA as described herein, or a composition comprising such an SMN2 saRNA or a nucleic acid encoding an SMN2 saRNA as described herein, in combination with a SMN2 mRNA modulator, for the preparation of one or more compositions for increasing the amount of full-length SMN protein expressed by a cell.

In another aspect, the invention provides an isolated SMN2 gene saRNA targeting site having any contiguous 16-35 nucleotide sequence on the promoter region of the SMN2 gene, preferably any contiguous 16-35 nucleotide sequence on any one of the sequences selected from the group consisting of SEQ ID NO: 472-475. In particular, the site of action comprises or is selected from the sequence shown in any of the nucleotide sequences of SEQ ID NO: 315-471.

Another embodiment provides pharmaceutical compositions or medicaments comprising the compounds of the invention and a therapeutically inert carrier, diluent or pharmaceutically acceptable excipient, as well as methods of using the compounds of the invention to prepare such compositions and medicaments. In certain embodiments, the SMN2 saRNA and SMN2 mRNA modulator of the invention are in separate pharmaceutical compositions. In other embodiments, the SMN2 saRNA and SMN2 mRNA modulator are in the same pharmaceutical composition.

Compositions of the present disclosure are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Compositions comprising any of the small molecule compounds described herein, for example, Risdiplam or Branaplam, may be administered separately from the SMN2 saRNA composition by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. For SMN2 saRNA compositions, the delivery can be through parenteral infusions including intrathecal, intramuscular, intravenous, intraarterial, intraperitoneal, or subcutaneous administration. In some embodiments, administration of the compositions of the present disclosure can be optionally through parenteral infusions including intrathecal, intramuscular, intravenous, intraarterial, intraperitoneal, intravesical, intracerebroventricular, intravitreal or subcutaneous administration; or through oral administration, intranasal administration, inhaled administration, vaginal administration, or rectal administration.

The small molecule compounds described herein, such as Risdiplam and Branaplam, may be administered in any convenient administrative form, e.g., tablets, powders, capsules, solutions, dispersions, suspensions, syrups, sprays, suppositories, gels, emulsions, patches, etc. Such compositions may comprise components conventional in pharmaceutical preparations, e.g., diluents, carriers, pH modifiers, preservatives, solubilizers, stabilizers, wetting agents, emulsifiers, sweeteners, colorants, flavorants, salts for varying the osmotic pressure, buffers, masking agents, antioxidants, and further active agents. Such compositions can also comprise still other therapeutically valuable substances.

A typical formulation is prepared by mixing a compound of the present invention and a carrier or excipient. Suitable carriers and excipients are well known to those skilled in the art and are described in detail in, e.g., Ansel H. C. et al., Ansel's Pharmaceutical Dosage Forms and Drug Delivery Systems (2004) Lippincott, Williams & Wilkins, Philadelphia: Gennaro A. R. et al., Remington: The Science and Practice of Pharmacy (2000) Lippincott, Williams & Wilkins, Philadelphia; and Rowe R. C, Handbook of Pharmaceutical Excipients (2005) Pharmaceutical Press, Chicago. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

In another aspect, the invention provides use of the combination according to any one of the embodiments described herein, or a composition according to any one of the embodiments described herein, in the manufacture of a medicament for the treatment of an SMN-deficiency-related condition in an individual. The use according to certain embodiments, the SMN-deficiency-related condition comprises a hereditary neuromuscular disease, preferably spinal muscular atrophy. Also provided is the use according to certain embodiments wherein the individual is a mammal, preferably a human.

6.2.2. SMN2 mRNA Modulators

The term “SMN2 mRNA modulator” as used herein refers to a modulator of SMN2 mRNA splicing or stability that increases the production of functional SMN2 mRNA and functional SMN protein. The term “SMN2 mRNA modulator” includes an agent that changes the way the SMN2 pre-mRNA is spliced so that it contains all the information necessary to make functional full-length SMN protein, for example, by blocking the effect of the intronic inhibitory splicing region of intron 7 of the SMN2 gene. An SMN2 mRNA modulator includes those agents that increase the desired splicing and subsequent protein production by stabilizing the interaction between the spliceosome and SMN2 pre-mRNA (J Med Chem, 2018 Dec. 27; 61(24): 11021-11036), and those agents that enhance stabilization of the transient double-strand RNA structure formed by the SMN2 pre-mRNA and U1 small nuclear ribonucleic protein (snRNP) complex (Nat Chem Biol, 2015 July; 11(7):511-7). In certain examples, an SMN2 mRNA modulator will modulate the splicing of SMN2 pre-mRNA to include exon 7 in the processed transcript. Alternatively, SMN2 mRNA modulators of the present disclosure include agents which possess the ability to increase functional SMN protein levels by preventing exon7 from being spliced out of the mature SMN mRNA during splicing. The SMN2 mRNA modulator in accordance with the present disclosure also includes those described in U.S. Pat. Nos. 10,436,802 and 10,420,753, the entirety of each of which are incorporated herein by reference.

Examples of SMN2 mRNA modulators in accordance with the present disclosure include pyridazine derivatives, for example those described in WO2014028459A1, the entire contents of which are incorporated herein by reference. Specific examples of SMN2 mRNA modulators in include Branaplam (also known as LMI070) and Risdiplam (also known as RG7916, or RO7034067).

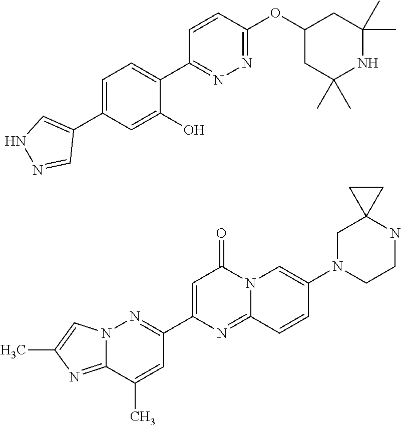

Further examples of SMN2 mRNA modulators in accordance with the present disclosure include antisense oligonucleotides such as those capable of antisense targeting, displacement and/or disruption of an intronic sequence in the SMN2 gene to enhance the production of SMN2 full-length (SMN2FL) transcripts (transcripts containing exon 7) during splicing. In certain embodiments, Nusinersen, marketed as Spinraza®, is suitable for use in accordance with the disclosed combinations.

### 6.3. Methods of Treating SMA and Related Conditions

Another aspect of the invention relates to a method of treating or delaying the onset of an SMN-deficiency-related condition in an individual comprising administering to the individual a therapeutically effective amount of an SMN2 saRNA as described herein, a nucleic acid encoding an SMN2 saRNA as described herein, or a composition comprising an SMN2 saRNA of the invention or a nucleic acid encoding an SMN2 saRNA as described herein. The subject may be a mammal, such as a human. The subject may be an infant, a child or an adult. In one embodiment, the disease caused by insufficient SMN full-length protein expression or SMN1 gene mutation may include, for example, SMA. In one embodiment, the disease caused by under-expression of the SMN full-length protein, mutation or deletion of the SMN1 gene, and/or under-expression of the full-length SMN protein is SMA. In one embodiment, the SMA of the present invention includes SMA Type I, SMA Type II, SMA Type III, and SMA Type IV.

Another aspect of the invention relates to the use of an SMN2 saRNA of the present disclosure, a nucleic acid encoding an SMN2 saRNA of the present disclosure or a composition comprising an SMN2 saRNA of the present disclosure or a nucleic acid encoding an SMN2 saRNA of the present disclosure in combination with an SMN2 mRNA modulator of the present disclosure for the preparation of a medicament for the treatment or delaying the onset of an SMN-deficiency-related condition. The subject may be a mammal, such as a human. The subject may be an infant, a child or an adult. In one embodiment, the an SMN-deficiency-related condition may include, for example, SMA. In one embodiment, the SMA of the present invention includes SMA Type I, SMA Type II, SMA Type III, and SMA Type IV.

Also provided is the use according to any one of the combinations of an SMN2 saRNA and an SMN2 mRNA modulator described herein, or a composition according to any one of the combinations of an SMN2 saRNA and an SMN2 mRNA modulator described herein, in the manufacture of a preparation for increasing the amount of full-length SMN protein in a cell. In certain embodiments, the cell is a mammalian cell, preferably a human cell. In certain embodiments, the cell is present in a human. In certain embodiments, the human is a patient suffering from symptoms caused by an SMN-deficiency-related condition. In certain embodiments, the combinations, or the compositions thereof, is administered in an amount an amount effective to treat the SMN-deficiency-related condition. In certain embodiments, the symptoms caused by SMN-deficiency-related condition are those associated with hereditary neuromuscular diseases, preferably spinal muscular atrophy.

In certain embodiments, the combination of an SMN2 saRNA and an SMN2 mRNA modulator achieves an increase in full-length SMN protein that is greater than the amount achieved by administration of the same amount of either substance used individually, with reduced toxicity or unwanted side effects. In certain embodiments, the combination of an SMN2 saRNA and an SMN2 mRNA modulator achieves an increase in full-length SMN protein that is greater than the additive effect of treatment with the same amount of either substance used individually. In certain embodiments, amount of SMN2 saRNA or SMN2 mRNA modulator administered in an amount that is less than the amount used for conventional treatment when used in an embodiment of the combination described herein.

In certain embodiments, the effect of the combination of an SMN2 saRNA and an SMN2 mRNA modulator achieves a greater clinical improvement compared to the effect of the same amount of either substance used individually. In certain embodiments, the effect of the combination of an SMN2 saRNA and an SMN2 mRNA modulator achieves a greater than additive clinical improvement compared to the effect of the same amount of either substance used individually.

The present disclosure also relates to a method of increasing the amount of full-length SMN protein in a cell comprising administering to the cell a combination of 1) an SMN2 mRNA modulator and 2) at least one of an SMN2 saRNA as described herein, a nucleic acid encoding an SMN2 saRNA as described herein, or a composition comprising the SMN2 saRNA or a nucleic acid encoding an SMN2 saRNA as described herein.

In any of the embodiments provided herein, such SMN2 saRNAs, nucleic acids encoding SMN2 saRNAs of the present disclosure, or compositions comprising such SMN2 saRNAs or nucleic acids encoding SMN2 saRNAs of the present disclosure may be introduced directly into a cell, or may be produced intracellularly upon introduction of a nucleotide sequence encoding the SMN2 saRNA into a cell, preferably a mammalian cell, more preferably a human cell. Such cells may be ex vivo, such as cell lines, and the like, or may be present in mammalian bodies, such as humans. In some embodiments, the human is a patient or individual suffering from a SMN-deficiency-related condition. In certain embodiments, a nucleic acid encoding an SMN2 saRNA or a composition comprising the aforementioned saRNA or a nucleic acid encoding an SMN2 saRNA of the invention is administered in combination with a composition comprising at least one SMN2 mRNA modulator, in respective amounts sufficient to effect treatment of the SMN-deficiency-related condition. In one embodiment, the SMN-deficiency-related condition is SMA. In one embodiment, the SMA of the present disclosure includes SMA Type I, SMA Type II, SMA Type III, and SMA Type IV.

In certain embodiments, the combination of SMN2 saRNA and SMN2 mRNA modulator achieves an increase in full-length SMN protein that is greater than the amount achieved by administration of the same amount of either substance used individually. In certain embodiments, the combination of SMN2 saRNA and SMN2 mRNA modulator has reduced toxicity and/or reduced unwanted side effects compared to treatment by monotherapy. In certain embodiments, the combination of SMN2 saRNA and SMN2 mRNA modulator achieves an increase in full-length SMN protein that is greater than the additive effect of treatment with the same amount of either substance used individually. In certain embodiments, either the SMN2 saRNA or the SMN2 mRNA modulator, or both are administered in an amount less than the amount that would be used for conventional monotherapy treatment.

In certain embodiments, the combination of the SMN2 saRNA and the SMN2 mRNA modulator achieves a greater clinical improvement compared to the effect of the same amount of either substance used individually. In certain embodiments, the combination of SMN2 saRNA and SMN2 mRNA modulator achieves a greater than additive clinical improvement compared to the effect of the same amount of either substance used individually.

In certain embodiments, the baseline measurement is obtained from a biological sample, as defined herein, obtained from an individual prior to administering the therapy described herein. In certain embodiments, the biological sample is peripheral blood mononuclear cells, blood plasma, serum, skin tissue, cerebrospinal fluid (CSF). In certain embodiments, increases in SMN protein levels in peripheral blood mononuclear cells and skin correlate with those in neurons of the central nervous system (CNS), indicating that a change of these levels in blood or skin can be used as a non-invasive surrogate to determine changes of SMN protein levels in the CNS. In further embodiments, the combination provided herein increases the amount of full-length SMN protein as compared to the baseline measurement, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 115%, by at least 120%, by at least 125%, by at least 130%, by at least 135%, by at least 140%, by at least 145%, by at least 150%, by at least 155%, by at least 160%, by at least 165%, by at least 170%, by at least 175%, by at least 180%, by at least 185%, by at least 190%, by at least 195%, by at least 200%, by at least 210%, by at least 215%, by at least 220%, by at least 225%, by at least 230%, by at least 235%, by at least 240%, by at least 245%, by at least 250%, by at least 255%, by at least 260%, by at least 265%, by at least 270%, by at least 275%, by at least 280%, by at least 285%, by at least 290%, by at least 295%, by at least 300%, by at least 310%, by at least 315%, by at least 320%, by at least 325%, by at least 330%, by at least 335%, by at least 340%, by at least 345%, by at least 350%, by at least 355%, by at least 360%, by at least 365%, by at least 370%, by at least 375%, by at least 380%, by at least 385%, by at least 390%, by at least 395%, by at least 400%.

In the context of the present disclosure, the term “co-administration” of the one or more SMN2 saRNAs and the one or more SMN2 mRNA modulators can be simultaneous, (i.e., within 15 minutes, within 30 minutes, or within an hour) almost simultaneous (i.e., within 2 hours, within 4 hours, within 6 hours, within 8 hours within 10 hours, or within 12 hours, within 24 hours), or delayed in time by a few days or weeks, for example by up to 4 or 5 weeks.

In the context of the present disclosure, the term “co-administration” of the composition comprising one or more SMN2 saRNAs and the composition comprising one or more SMN2 mRNA modulators can be simultaneous or administered at the same time, (i.e., within 15 minutes, within 30 minutes, within an hour,) almost simultaneous or roughly the same time (i.e., within 2 hours, within 4 hours, within 6 hours, within 8 hours within 10 hours, within 12 hours, within 24 hours), or delayed in time by a few days or weeks, for example by up to 4 or 5 weeks.

The dosage at which compositions of the present disclosure can be administered can vary within wide limits and will, of course, be fitted to the individual requirements in each case.

In a particular embodiment, the combination of SMN2 saRNA and SMN2 mRNA modulator show a greater than additive effect or synergy in the treatment, prevention, delaying progression and/or amelioration of diseases caused by an inactivating mutation or deletion in the SMN1 gene and/or associated with loss or defect of SMN1 gene function, and additionally for the protection of cells implicated in the pathophysiology of the disease, particularly for the treatment, prevention, delaying progression and/or amelioration of spinal muscular atrophy (SMA).

In certain embodiments, a first dose of a pharmaceutical composition according to the present disclosure is administered when the subject is less than one week old, less than one month old, less than 3 months old, less than 6 months old, less than one year old, less than 2 years old, less than 15 years old, or older than 15 years old.

In certain embodiments, at least one pharmaceutical composition comprising the SMN2 saRNA and at least one other pharmaceutical composition comprising the SMN2 mRNA modulator are co-administered simultaneously, almost simultaneously or are co-administered at different times. In certain embodiments the pharmaceutical composition comprising the SMN2 mRNA modulator and the pharmaceutical composition comprising the SMN2 saRNA are co-administered within one hour of each other, within two hours of each other, within three hours of each other, within four hours of each other, within five hours of each other, within six hours of each other, within seven hours of each other, within eight hours of each other, within nine hours of each other, within 10 hours of each other, within 11 hours of each other, within 12 hours of each other, within one day of each other, within two days of each other, within three days of each other, within four days of each other, within five days of each other, within six days of each other, within one week of each other, within two weeks of each other, within three weeks of each other, within four weeks of each other, or within 5 weeks of each other. The single dose can be of SMN2 saRNA, and can be a single 0.1 to 15 milligram dose, a single 1 milligram dose, a single 2 milligram dose, a single 3 milligram dose, a single 4 milligram dose, a single 5 milligram dose, a single 6 milligram dose, single 7 milligram dose, a single 8 milligram dose, a single 9 milligram dose, a single 10 milligram dose, a single 11 milligram dose, a single 12 milligram dose, a single 13 milligram dose, a single 14 milligram dose, or a single 15 milligram dose. The single dose can be of SMN2 mRNA modulator, and can be a single 0.1 to 15 milligram dose, a single 1 milligram dose, a single 2 milligram dose, a single 3 milligram dose, a single 4 milligram dose, a single 5 milligram dose, a single 6 milligram dose, single 7 milligram dose, a single 8 milligram dose, a single 9 milligram dose, a single 10 milligram dose, a single 11 milligram dose, a single 12 milligram dose, a single 13 milligram dose, a single 14 milligram dose, or a single 15 milligram dose.

In certain embodiments, a single 4.8 milligram dose of the SMN2 mRNA modulator is an ASO and is administered as an intrathecal injection by lumbar puncture. In certain embodiments, the SMN2 mRNA modulator is Nusinersen. In certain embodiments, it can be a single 5.16 milligram dose, a single 5.40 milligram dose, a single 7.2 milligram dose, a single 7.74 milligram dose, a single 8.10 milligram dose, a single 9.6 milligram dose, a single 10.32 milligram dose, a single 10.80 milligram dose, a single 11.30 milligram dose, a single 12 milligram dose, a single 12.88 milligram dose, a single 13.5 milligram dose, a single 14.13 milligram dose, a single 10 milligram dose, a single 11 milligram dose, a single 12 milligram dose, a single 13 milligram dose, a single 14 milligram dose, a single 15 milligram dose, a single 16 milligram dose, a single 17 milligram dose, a single 18 milligram dose, a single 19 milligram dose, or a single 20 milligram dose.

In certain embodiments, where a dose of SMN2 saRNA and/or SMN2 mRNA is administered as an intrathecal injection by lumbar puncture, the use of a smaller gauge needle may reduce or ameliorate one or more symptoms associated with a lumbar puncture procedure. In certain embodiments, symptoms associated with a lumbar puncture include, but are not limited to, post-lumbar puncture syndrome, headache, back pain, pyrexia, constipation, nausea, vomiting, and puncture site pain. In certain embodiments, use of a 24- or 25-gauge needle for the lumbar puncture reduces or ameliorates one or more post lumbar puncture symptoms. In certain embodiments, use of a 21-, 22-, 23-, 24- or 25-gauge needle for the lumbar puncture reduces or ameliorates post-lumbar puncture syndrome, headache, back pain, pyrexia, constipation, nausea, vomiting, and/or puncture site pain.

Proposed dose frequency is approximate, for example, in certain embodiments if the proposed dose frequency is a dose at day 1 and a second dose at day 29, an SMA patient may receive a second dose 25, 26, 27, 28, 29, 30, 31, 32, 33, or 34 days after receipt of the first dose. In certain embodiments, if the proposed dose frequency is a dose at day 1 and a second dose at day 15, an SMA patient may receive a second dose 10, 11, 12, 13, 14, 15, 16, 17, 18, 19, or 20 days after receipt of the first dose. In certain embodiments, if the proposed dose frequency is a dose at day 1 and a second dose at day 85, an SMA patient may receive a second dose 80, 81, 82, 83, 84, 85, 86, 87, 88, 89, or 90 days after receipt of the first dose.

In certain embodiments, the dose and/or the volume of the injection will be adjusted based on the patient's age, the patient's CSF volume, or the patient's age and/or estimated CSF volume. (For example, see Matsuzawa J, Matsui M, Konishi T, Noguchi K, Gur R C, Bilker W, Miyawaki T. Age-related volumetric changes of brain gray and white matter in healthy infants and children. Cereb Cortex 2001 April; 11(4):335-342, which is hereby incorporated by reference in its entirety).

### 7. EXAMPLES

The present invention is further illustrated by the following examples. These examples are provided merely for illustration purposes and shall not be interpreted to limit the scope or content of the present invention in any way.

### 7.1. Example 1: Effect of saRNA (DS06-0004), ASO (Nusinersen) and Risdiplam on the Expression of Full-Length and Exon 7 Skipped SMN2 mRNA in GM03813 Cells

In order to determine the optimal concentration of saRNA (DS06-0004), Nusinersen (ASO-10-27) and Risdiplam for cell treatment, saRNA and ASO were individually transfected at different concentrations into GM03813 cells. Risdiplam was dissolved in DMSO and added at different concentrations to cultured GM03813 cells.

“GM03813 cells” refers to fibroblast cells provided by Coriell Institute for Medical Research. This cell line is described as SPINAL MUSCULAR ATROPHY, TYPE II; SMA2 SURVIVAL OF MOTOR NEURON 1, TELOMERIC; SMN1. The relevant gene is SMN1; the chromosomal location is 5q12.2-q13.3, the allelic variant is described as 1 exons 7 and 8 deleted, SPINAL MUSCULAR ATROPHY, TYPE I; and the identified mutation: is EX7-8DEL. The phenotype data derived from a fibroblast from skin (arm) of the following subject characterized as: clinically affected; born after full term uncomplicated pregnancy; rolled over at 6 months old; began babbling at 9 months old; by 12 months old, there was marked muscle atrophy and weakness; absent deep tendon reflexes; constipation; donor subject has 3 copies of the SMN2 gene; PCR analysis showed that this donor subject is homozygous for the deletion of exons 7 and 8 in the SMN1 gene; similarly affected brother (not in repository); mother is GM03814 (Fibroblast)/GM24474 (iPSC); father is GM03815 (Fibroblast); see GM23240 (iPSC—lentiviral) and GM24468 (iPSC—episomal); previously classified as SMA I, but data such as onset features and SMN2 dosage in the proband supported re-classification to SMA II.

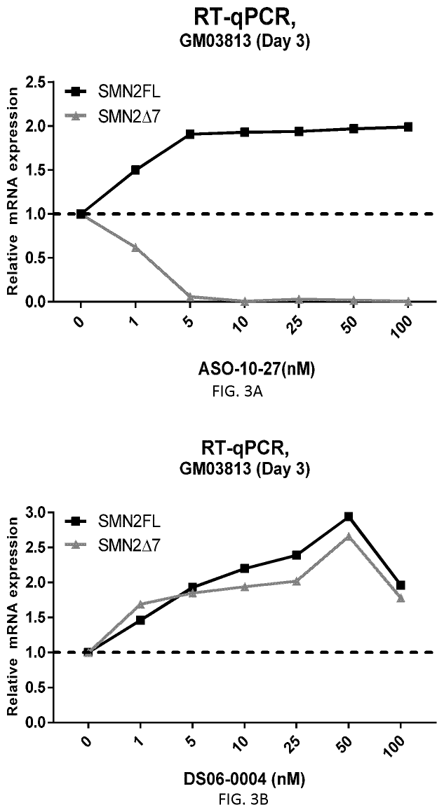

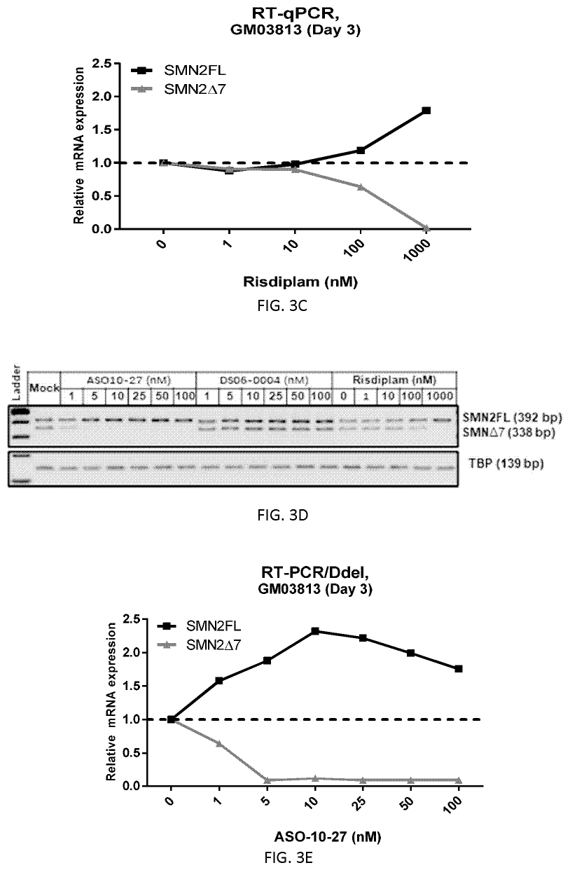

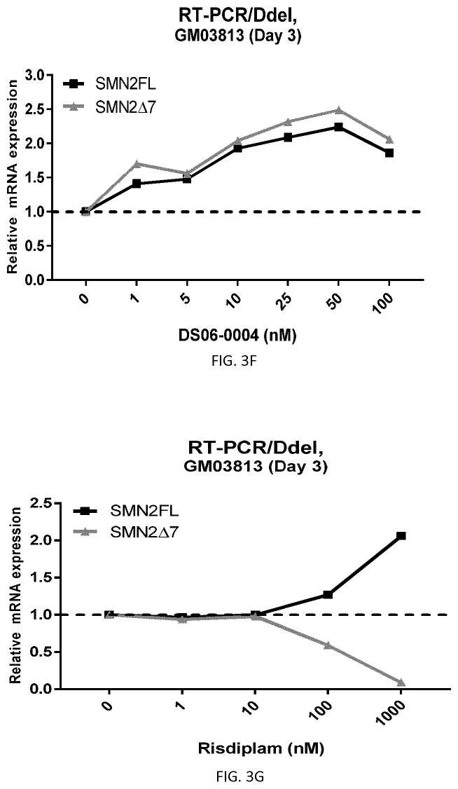

Seventy-two hours later, total cellular RNA was isolated from the treated cells and reverse transcribed into cDNA. SMN2 mRNA expression was assessed with RT-qPCR using primer pairs specific for SMN2FL or SMN2 Δ7. SMN2 mRNA expression was also assessed by semi-quantitative RT-PCR using a primer pair that amplifies both SMN2FL and SMN2 Δ7 followed by DdeI digestion (PCR/digestion). The PCR resulted in two product bands: 507 bp (SMN2FL) and 453 bp (SMN2 Δ7). After digestion, both bands were reduced by 115 bp, resulting in two products: 392 bp (SMN2FL) and 338 bp (SMN2 Δ7) as shown on the gel of FIG. 3D. FIG. 3A-3D show dose-dependent changes in SMN2FL and SMNΔ7 mRNA as assessed by RT-qPCR and PCR/digestion respectively. FIG. 3E-3G are graphic plots of data from quantitating the intensity of bands in FIG. 3D.

As shown in FIG. 3A, ASO-10-27 treatment at 1 nM increased SMN2FL by 1.5-fold and at 5 nM caused a peak increase of 2.0-fold with a concurrent decrease in SMN2 Δ7, while higher doses did not cause further induction of SMN2FL or reduction of SMN2 Δ7. Similarly, PCR/digestion analysis shows that the expression of SMN2FL reached its peak when cells were treated with ASO at 10 nM, and the expression of SMNΔ7 almost approached the lowest value at 5 nM (FIG. 3E). Risdiplam treatment at 100 nM and 1000 nM increased the mRNA level of SMN2FL by 1.2 and 1.8-fold and decreased SMN2 Δ7 by 36% and 98% respectively (FIGS. 3C and 3G). Since SMN2 mRNA modulators including ASO-10-27 and Risdiplam increase SMN2FL mRNA by modulating SMN2 splicing to include more exon 7, the maximum amount of SMN2FL they can induce depends on the available amount of SMN2 pre-mRNA which is not altered by the SMN2 mRNA modulators. Consistent with this view, the data shows a ceiling effect on splicing modulator-induced SMN2FL increase (maximum increase at ˜2 fold) by ASO-10-27 and Risdiplam.

In contrast, saRNA (DS06-0004) induced the expression of both SMN2FL and SMN2 Δ7 to a higher level than SMN2 mRNA modulators and in a dose-dependent manner at concentrations ranging from 1 nM to 50 nM with the highest fold change being 2.9- and 2.7-fold respectively. DS06-0004 at 100 nM did not further increase SMN2 mRNA expression (FIGS. 3B and 3F). Consistent results were obtained by PCR/digestion analysis (FIG. 3D and FIG. 3F).

Different from the SMN2 mRNA modulators (ASO-10-27 and Risdiplam), which increased the levels of SMN2FL by converting (reducing) SMN2 Δ7 levels, the SMN2 saRNAs of the present disclosure increase SMN2 mRNA levels by acting on SMN2 transcription, resulting in a concurrent increase in both SMN2FL and SMN2 Δ7. The data revealed in FIG. 3 clearly demonstrates the mechanistic differences between SMN2 mRNA modulators and SMN2 saRNAs.

### 7.2. Example 2: Combinatory Effect of saRNA (DS06-0004) and ASO-10-27 on the Expression of Full-Length and Exon 7 Skipped SMN2 mRNA in GM00232 Cells

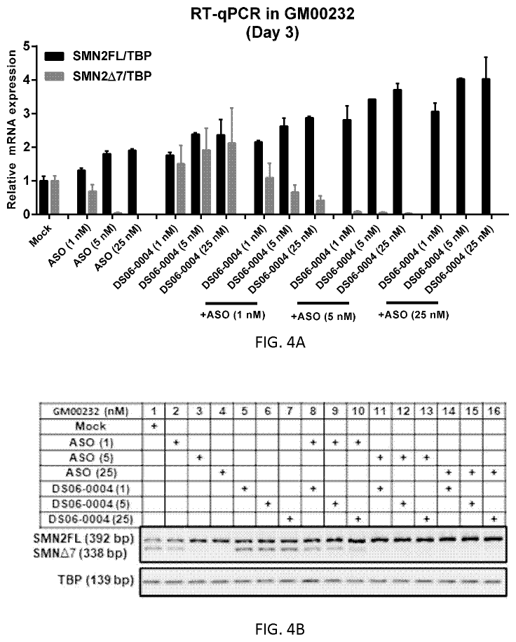

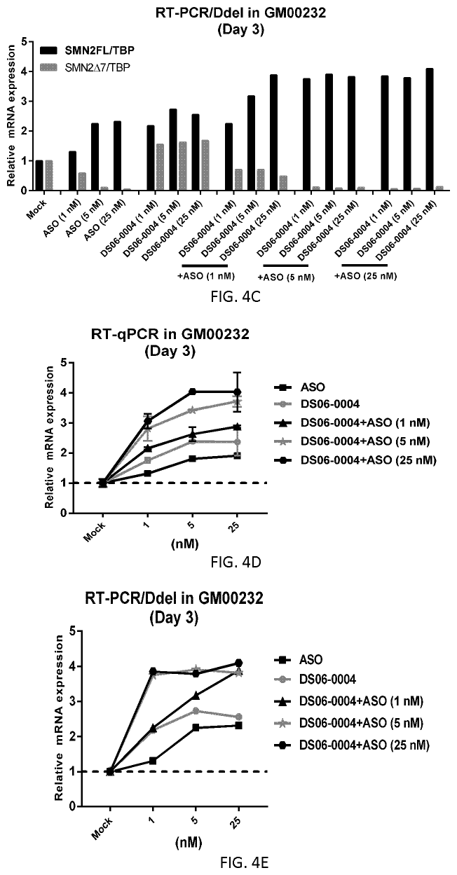

To determine whether the combination of saRNA (DS06-0004) and ASO-10-27 has an enhanced effect on SMN2FL induction in type I SMA cells, GM00232 cells were transfected with DS06-0004 and ASO-10-27 alone or in combination at different concentrations for 72 hours. SMN2 expression was assessed in the treated cells by RT-qPCR (FIGS. 4A and 4D) and PCR/digestion (FIGS. 4B, 4C and 4E).

“GM00232 cells” refers to fibroblast cells provided by Coriell Institute for Medical Research. This cell line is described as SPINAL MUSCULAR ATROPHY I; SMA1. The donor subject has 2 copies of the SMN2 gene (data from several sources including Stabley et al. 2015, PMID 26247043) and is homozygous for deletion of exons 7 and 8 of the SMN1 gene. The relevant gene is SMN1; the chromosomal location is 5q12.2-q13.3, the allelic variant is described as exons 7 and 8 deleted; SPINAL MUSCULAR ATROPHY, TYPE I; and the identified mutation: is EX7-8DEL. The phenotype data derived from a fibroblast from skin (arm) of the following subject characterized as: Progressive muscular atrophy; absent deep tendon reflexes; abnormal EMG; donor subject has 2 copies of the SMN2 gene (data from several sources including Stabley et al. 2015, PMID 26247043) and is homozygous for deletion of exons 7 and 8 of the SMN1 gene.

As shown in FIG. 4A, ASO-10-27 at 1 nM, 5 nM and 25 nM caused a 1.3-, 1.8- and 1.9-fold increase in SMN2FL respectively with concurrent decrease of SMN2 Δ7. DS06-0004 at 1 nM, 5 nM and 25 nM increased SMN2FL by 1.7, 2.4 and 2.4-fold, respectively, and increased SMN2 Δ7 by 1.5, 1.9 and 2.1-fold, respectively.

When 1 nM of ASO-10-27 was combined with increasing concentrations of DS06-0004 (1 nM, 5 nM and 25 nM) in cell transfection, SMN2FL was induced by 2.2-, 2.6- and 2.9-fold, respectively, while SMN2 Δ7 was changed by 1.1-, 0.7- and 0.4-fold, respectively. Further, treating cells with 5 nM of ASO-10-27 in combination with increasing concentrations of DS06-0004 (1 nM, 5 nM and 25 nM) induced SMN2FL by 2.8-, 3.4- and 3.7-fold, respectively and changed SMN2 Δ7 by 0.09-, 0.05- and 0.04-fold, respectively. Furthermore, treating cells with 25 nM of ASO-10-27 in combination with increasing concentrations of DS06-0004 (I nM, 5 nM and 25 nM) induced SMN2FL by 3.1-, 4.0- and 4.0-fold, respectively, and completely eliminated SMN2 Δ7 expression. Compared to 25 nM ASO-10-27 treatment alone which induced SMN2FL by 1.9-fold, combinatory treatment of ASO-10-27 and DS06-0004 increased SMN2FL by 4-fold, doubling the effect of ASO-10-27 when it was used alone.

The RT-qPCR result presented in FIG. 4A was further verified PCR/DdeI digestion. Consistent with RT-qPCR result, ASO-10-27 alone caused a 2.3-fold increase in SMN2FL mRNA at 25 nM and combination of ASO-10-27 (25 nM) and DS06-0004 (25 nM) caused the highest induction of SMN2FL (4.1 fold) and a concurrent reduction in SMN2 Δ7 (0.14 fold) (FIGS. 4B, 4C and 4E).

Together, the data presented in FIG. 4 demonstrate that saRNA DS06-0004 alone had strong activity in inducing SMN2 mRNA expression, especially the expression of SMN2FL in type I SMA cells which have 2 copies of SMN2 gene. When the SMN2 saRNA was combined with ASO-10-27, maximum induction of SMN2FL could be achieved.

### 7.3. Example 3: Combinatory Effect of saRNA (DS06-0004) and ASO-10-27 on SMN Protein Levels in GM00232 Cells

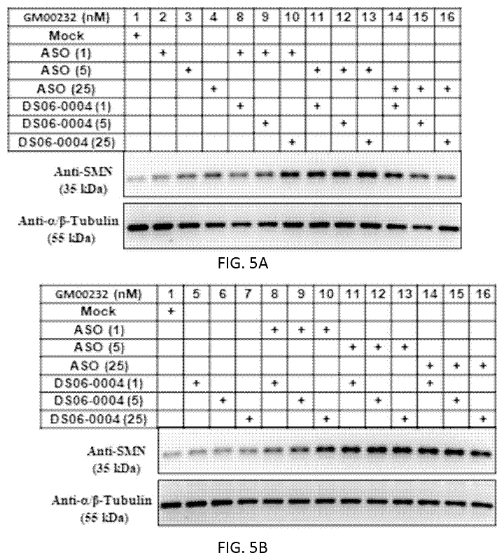

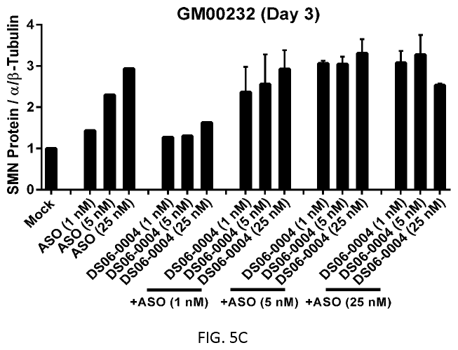

To further verify the effect of ASO-10-27 and DS06-0004 alone or in combination on SMN2 gene expression, western blotting assays were performed in GM00232 cells transfected individually or in combination with ASO-10-27 and DS06-0004. As shown in FIGS. 5A and 5C, ASO-10-27 at 1 nM, 5 nM and 25 nM caused a 1.4-, 2.3- and 2.9-fold increase in SMN protein, respectively. DS06-0004 at 1 nM, 5 nM and 25 nM increased SMN2FL by 1.2, 1.3 and 1.7 fold, respectively (FIGS. 5B and 5C). The protein bands with an expected size of 35 kDa are full-length SMN protein (FIGS. 5A and 5B), while SMNΔ7 protein does not appear on western blots because it is rapidly degraded (Le, T. T., et al. SMNDelta7, the major product of the centromeric survival motor neuron (SMN2) gene, extends survival in mice with spinal muscular atrophy and associates with full-length SMN. Human Mol Genet (2005).

Treating cells with 1 nM of ASO-10-27 in combination with increasing concentrations of DS06-0004 (1 nM, 5 nM and 25 nM) induced SMN protein by 2.4-, 2.6-, and 2.9-fold, respectively (FIGS. 5A-5C, 5A and 5B contain duplicates of combination treatments).

Further treating cells with 5 nM of ASO-10-27 in combination with increasing concentrations of DS06-0004 (1 nM, 5 nM and 25 nM) induced SMN protein by 3.1-, 3.0-, and 3.3-fold, respectively (FIGS. 5A-5C, 5A and 5B contain duplicates of combination treatments).

Furthermore, treating cells with 25 nM of ASO-10-27 in combination with increasing concentrations of DS06-0004 (1 nM, 5 nM and 25 nM) induced SMN protein by 3.1-, 3.3-, and 2.6-fold, respectively (FIGS. 5A-5C, 5A and 5B contain duplicates of combination treatments).

Together, these data confirm that combining ASO-10-27 and DS06-0004 could induce higher levels of SMN protein than either of them used individually.

### 7.4. Example 4: Combinatory Effect of saRNA (DS06-0004) and ASO-10-27 on the Expression of Full-Length and Exon 7 Skipped SMN2 mRNA in GM03813 Cells

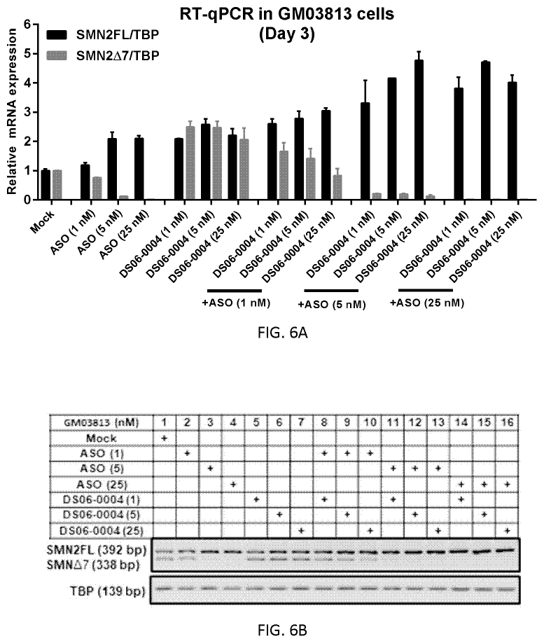

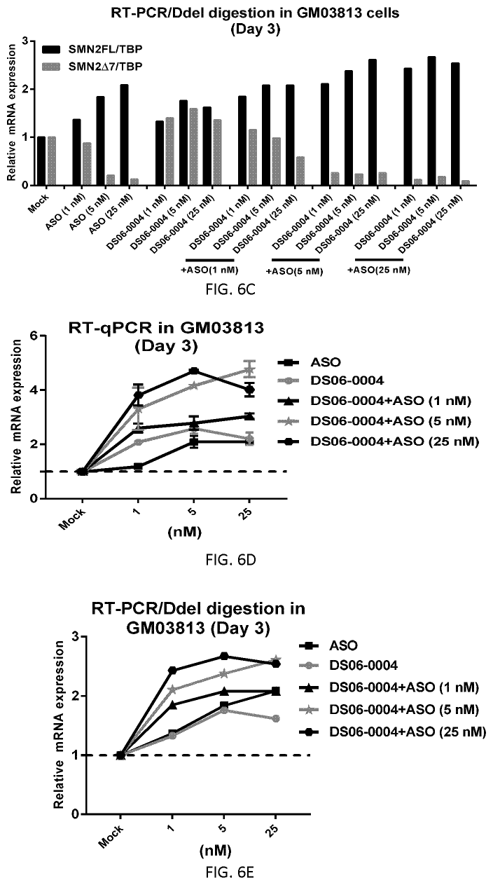

To determine whether the combination of saRNA (DS06-0004) and ASO-10-27 has an enhanced effect on SMN2FL induction in type II SMA cells, GM03813 cells were transfected with DS06-0004 and ASO-10-27 alone or in combination at different concentrations for 72 hours and SMN2 expression was assessed in the treated cells by RT-qPCR (FIGS. 6A and 6D) and PCR/digestion (FIGS. 6B, 6C and 6E). As shown in FIGS. 6A and 6D, ASO-10-27 at 1 nM, 5 nM and 25 nM caused a 1.2-, 2.1- and 2.1-fold increase in SMN2FL, respectively with concurrent decrease of SMN2 Δ7. DSO6-0004 at 1 nM, 5 nM and 25 nM increased SMN2FL by 2.1, 2.6 and 2.2-fold respectively and SMN2 Δ7 by 2.5, 2.5 and 2.1-fold, respectively.

As shown in FIGS. 6A and 6D, treating cells with 1 nM of ASO-10-27 in combination with increasing concentrations of DS06-0004 (1 nM, 5 nM and 25 nM) induced SMN2FL by 2.6-, 2.8- and 3.0-fold, respectively and SMN2 Δ7 by 1.7-, 1.4- and 0.8-fold, respectively. Further, treating cells with 5 nM of ASO-10-27 in combination with increasing concentrations of DS06-0004 (1 nM, 5 nM and 25 nM) induced SMN2FL by 3.3-, 4.2- and 4.8-fold, respectively and SMN2 Δ7 by 0.2-, 0.2- and 0.1-fold, respectively. Furthermore, treating cells with 25 nM of ASO-10-27 in combination with increasing concentrations of DS06-0004 (1 nM, 5 nM and 25 nM) induced SMN2FL by 3.8-, 4.7- and 4.0-fold, respectively and completely eliminated SMN2 Δ7 expression. Compared to 25 nM ASO-10-27 treatment alone which induced SMN2FL by 2.1-fold, combinatory treatment with ASO-10-27 and DS06-0004 increased SMN2FL by 4.7-fold, more than doubling the effect of ASO-10-27 when it was used alone.

The RT-qPCR result presented in FIG. 6A was further verified by semi-quantitative RT-PCR followed by DdeI digestion. Consistent with RT-qPCR result, ASO-10-27 alone caused a 2.1-fold increase in SMN2FL mRNA at 25 nM and combination of ASO-10-27 (25 nM) and DS06-0004 (5 nM) caused the highest induction of SMN2FL (2.7 fold) and a concurrent reduction in SMN2 Δ7 (0.18 fold) (FIGS. 6B, 6C and 6E).

Together, the data presented in FIG. 6 demonstrate that SMN2 saRNA DS06-0004 alone had strong activity in inducing SMN2 mRNA expression, especially the expression of SMN2FL in type II SMA cells, which have 3 copies of SMN2 gene. When the SMN2 saRNA was combined with ASO-10-27, maximum induction of SMN2FL was achieved. This data confirms that the combination of ASO-10-27 and DS06-0004 induces a higher level of SMN protein in type II SMA cells (GM03813 cells), compared to the levels of SMN protein in the same type II SMA cells (GM03813 cells) induced by treatment by either agent individually. This data also establishes the level of SMN protein induced in the cells treated with a combination in accordance with the present disclosure, compared to a population of untreated GM03813 cells. As described herein, GM03813 cells have two copies of SMN2 and are utilized as a model for SMA.

### 7.5. Example 5: Combinatory Effect of saRNA (DS06-0004) and ASO-10-27 on SMN Protein Levels in GM03813 Cells

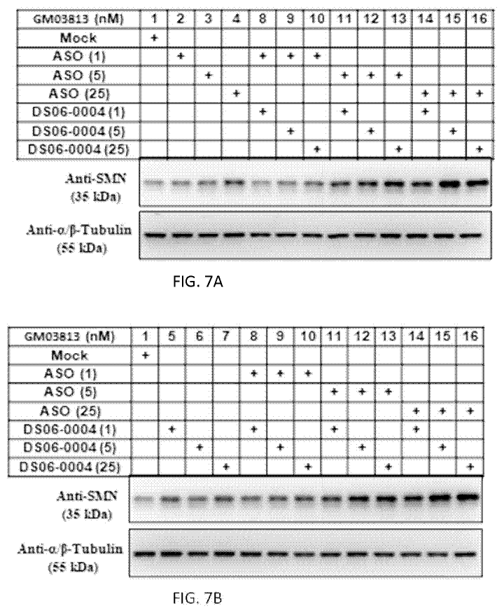

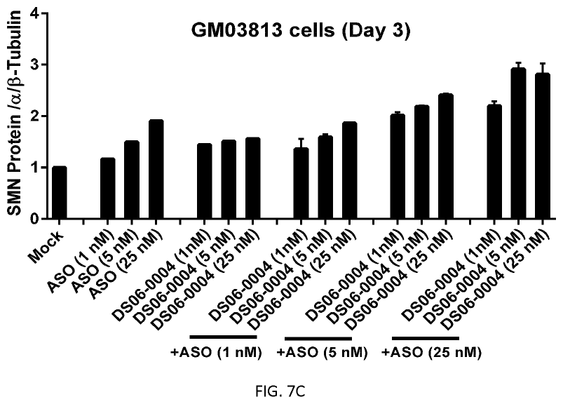

To further verify the effect of ASO-10-27 and DS06-0004 alone or in combination on SMN2 gene expression, western blotting assays were performed in GM03813 cells transfected individually or in combination with ASO-10-27 and DS06-0004. As shown in FIGS. 7A and 7C, ASO-10-27 at 1 nM, 5 nM and 25 nM caused a 1.2-, 1.5- and 1.9-fold increase in SMN protein, respectively. DS06-0004 at 1 nM, 5 nM and 25 nM increased SMN2FL by 1.5-, 1.5- and 1.6-fold, respectively (FIGS. 7B and 7C).

Treating cells with 1 nM of ASO-10-27 in combination with increasing concentrations of DS06-0004 (1 nM, 5 nM and 25 nM) induced SMN protein by 1.4-, 1.6-, and 1.8-fold, respectively (FIGS. 7A-7C, 7A and 7B contain duplicates of combo treatments).

Further treating cells with 5 nM of ASO-10-27 in combination with increasing concentrations of DS06-0004 (1 nM, 5 nM and 25 nM) increased SMN protein by 2.0-, 2.2-, and 2.4-fold, respectively (FIGS. 7A-7C, 7A and 7B contain duplicates of combo treatments).

Furthermore, treating cells with 25 nM of ASO-10-27 in combination with increasing concentrations of DS06-0004 (1 nM, 5 nM and 25 nM) increased SMN protein by 2.2-, 2.9-, and 2.8-fold, respectively (FIGS. 7A-7C, 7A and 7B contain duplicates of combo treatments).

Together, this data confirms that combining ASO-10-27 and DS06-0004 induces a higher level of SMN protein in type II SMA cells. This data confirms that the combination of ASO-10-27 and DS06-0004 induces a higher level of SMN protein in type II SMA cells (GM03813 cells), compared to the levels of SMN protein in the same type II SMA cells (GM03813 cells) induced by treatment by either agent individually. This data also establishes the level of SMN protein induced in the cells treated with a combination in accordance with the present disclosure, compared to a population of untreated GM03813 cells. As described herein, GM03813 cells have two copies of SMN2 and are utilized as a model for SMA.

### 7.6. Example 6: Combinatory Effect of saRNA (DS06-0004) and Risdiplam on the Expression of Full-Length and Exon 7 Skipped SMN2 mRNA in Type I SMA GM00232 Cells

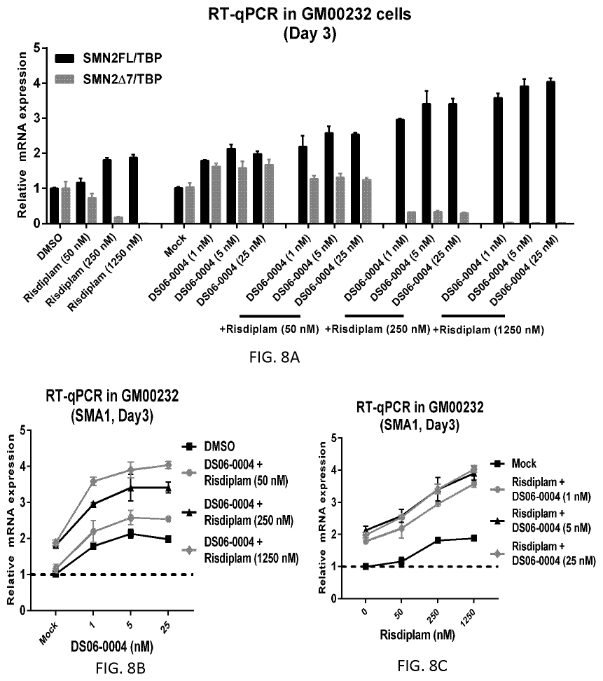

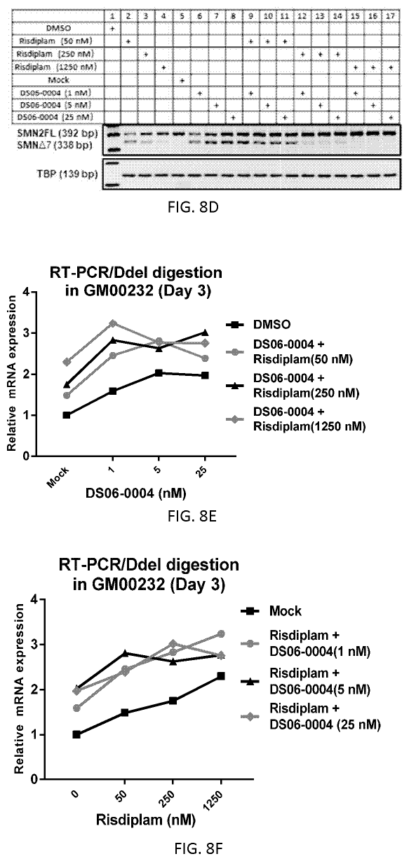

To determine whether the combination of an SMN2 saRNA and a small molecule SMN2 mRNA modulator, Risdiplam, has an enhanced effect on SMN2FL induction in type I SMA cells, GM00232 cells were treated with DS06-0004 and Risdiplam individually or in combination at different concentrations for 72 hours. SMN2 mRNA expression was assessed in the treated cells by RT-qPCR (FIG. 8A-8C) and PCR/digestion (FIG. 8D-8F). As shown in FIG. 8A-8C, Risdiplam at 50 nM, 250 nM and 1250 nM caused a 1.2-, 1.8- and 1.9-fold increase in SMN2FL, respectively with concurrent decrease of SMN2 Δ7. DS06-0004 at 1 nM, 5 nM and 25 nM increased SMN2FL by 1.8-, 2.1- and 2.0-fold, respectively and increased SMN2 Δ7 by 1.6-, 1.6- and 1.7-fold, respectively.

Treating cells with 50 nM of Risdiplam in combination with increasing concentrations of DS06-0004 (1 nM, 5 nM and 25 nM) increased SMN2FL by 2.2-, 2.6- and 2.5-fold, respectively and increased SMN2 Δ7 by 1.3-, 1.3- and 1.2-fold respectively. Further, treating cells with 250 nM of Risdiplam in combination with increasing concentrations of DS06-0004 (1 nM, 5 nM and 25 nM) increased SMN2FL by 3.0-, 3.4- and 3.4-fold, respectively, and changed SMN2 Δ7 by 0.3-, 0.3- and 0.3-fold, respectively. Furthermore, treating cells with 1250 nM of Risdiplam in combination with increasing concentrations of DS06-0004 (1 nM, 5 nM and 25 nM) increased SMN2FL by 3.6-, 3.9- and 4.0-fold, respectively, and completely eliminated SMN2 Δ7 expression. Compared to 1250 nM Risdiplam treatment alone which induced SMN2FL by 1.9-fold, combinatory treatment with Risdiplam and DS06-0004 increased SMN2FL by 4-fold, more than doubling the effect of Risdiplam when it was used alone.

The RT-qPCR result presented in FIG. 8A was further verified by PCR/DdeI digestion. Consistent with RT-qPCR result, Risdiplam alone caused a 2.3-fold increase in SMN2FL mRNA at 1250 nM and combination of Risdiplam (1250 nM) and DS06-0004 (1 nM) caused the maximum observed induction of SMN2FL (3.2 fold) (FIG. 8D-8F).

Together, the data presented in FIG. 8 demonstrates that saRNA DS06-0004 alone has strong activity in inducing SMN2 mRNA expression especially SMN2FL expression in type I SMA cells. When the SMN2 saRNA was combined with Risdiplam, maximum induction of SMN2FL was achieved.

### 7.7. Example 7: Combinatory Effect of saRNA (DS06-0004) and Risdiplam on SMN Protein Levels in GM00232 Cells

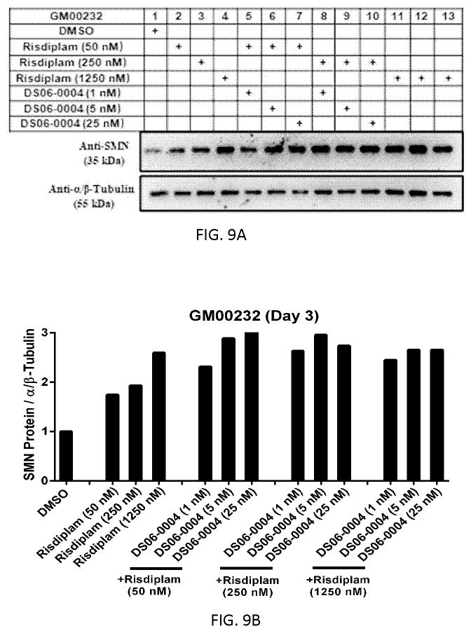

To further verify the effect of Risdiplam and DS06-0004, alone or in combination, on SMN2 gene expression in type I SMA cells GM00232, western blotting assays were performed in GM00232 cells transfected with Risdiplam alone and in combination with saRNA DS06-0004. As shown in FIGS. 9A and 9B, Risdiplam at 50 nM, 250 nM and 1250 nM caused a 1.7-, 1.9- and 2.6-fold increase in SMN protein, respectively.

Treating cells with 50 nM of Risdiplam in combination with increasing concentrations of DS06-0004 (1 nM, 5 nM and 25 nM) induced SMN protein by 2.3-, 2.9-, and 3.3-fold, respectively (FIGS. 9A and 9B).

Further treating cells with 250 nM of Risdiplam in combination with increasing concentrations of DS06-0004 (1 nM, 5 nM and 25 nM) induced SMN protein by 2.6-, 2.9-, and 2.7-fold, respectively (FIGS. 9A and 9B).

Furthermore, treating cells with 1250 nM of Risdiplam in combination with increasing concentrations of DS06-0004 (1 nM, 5 nM and 25 nM) induced SMN protein by 2.4-, 2.7-, and 2.7-fold, respectively (FIGS. 9A and 9B).

Together, these data confirm that the combinatory effect of saRNA and Risdiplam in increasing SMN2 expression can be verified at the protein level.

### 7.8. Example 8: Combinatory Effect of saRNA (DS06-0004) and Risdiplam on the Expression of Full-Length and Exon 7 Skipped SMN2 mRNA in Type II SMA GM03813 Cells

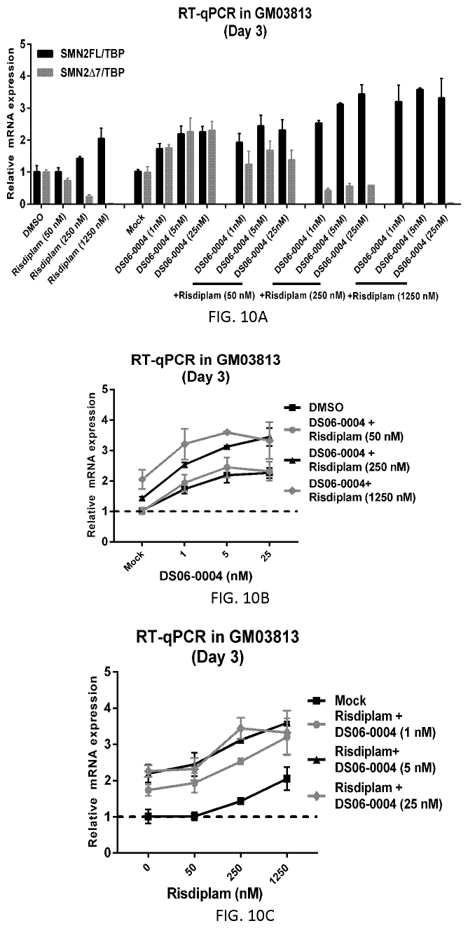

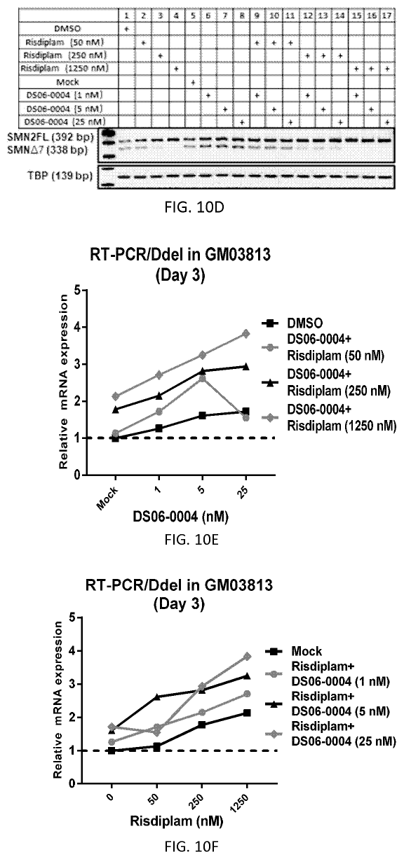

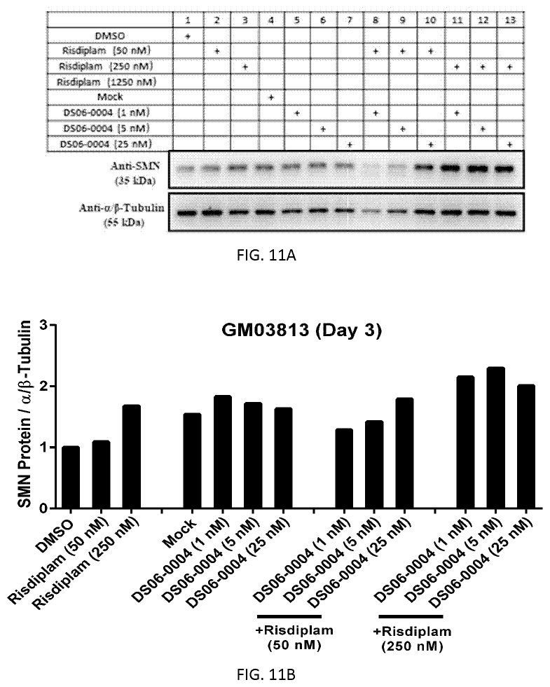

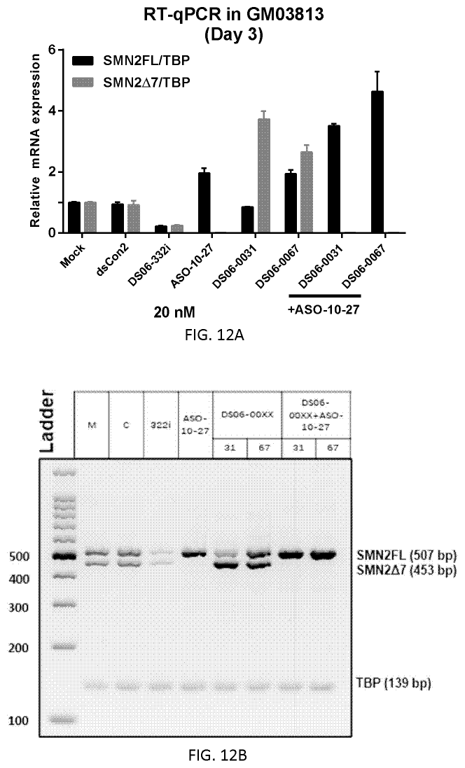

To determine whether the combination of saRNA and Risdiplam has an enhanced effect on SMN2FL induction in type II SMA cells, GM03813 cells were transfected with DS06-0004 and Risdiplam alone or in combination at different concentration for 72 hours and SMN2 mRNA expression was assessed in the treated cells by RT-qPCR (FIGS. 10A-10C) and PCR/digestion (FIG. 10D-10F). As shown in FIG. 10A-10C, Risdiplam at 50 nM, 250 nM and 1250 nM caused a 1.0-, 1.4- and 2.1-fold increase in SMN2FL, respectively with concurrent decrease of SMN2 Δ7. DS06-0004 at 1 nM, 5 nM and 25 nM increased SMN2FL by 1.7-, 2.2- and 2.3-fold, respectively, and increased SMN2 Δ7 by 1.8-, 2.3- and 2.3-fold, respectively.

Treating cells with 50 nM of Risdiplam in combination with increasing concentrations of DS06-0004 (1 nM, 5 nM and 25 nM) induced SMN2FL by 1.9-, 2.5- and 2.3-fold respectively and SMN2 Δ7 by 1.2-, 1.7- and 1.4-fold, respectively. Further treating cells with 250 nM of Risdiplam in combination with increasing concentrations of DS06-0004 (1 nM, 5 nM and 25 nM) induced SMN2FL by 2.5-, 3.1- and 3.4-fold, respectively and SMN2 Δ7 by 0.4-, 0.6- and 0.6-fold, respectively. Furthermore, treating cells with 1250 nM of Risdiplam in combination with increasing concentrations of DS06-0004 (1 nM, 5 nM and 25 nM) induced SMN2FL by 3.2-, 3.6- and 3.3-fold, respectively and completely eliminated SMN2 Δ7 expression. Compared to 1250 nM Risdiplam treatment alone which induced SMN2FL by 2.1-fold, combinatory treatment with Risdiplam and DS06-0004 increased SMN2FL by 3.6-fold, almost doubling the effect of Risdiplam when it was used alone.

The RT-qPCR result presented in FIG. 10A was further verified by PCR/DdeI digestion. Consistent with RT-qPCR result, Risdiplam alone caused a 2.1-fold increase in SMN2FL mRNA at 1250 nM and combination of Risdiplam (1250 nM) and DS06-0004 (25 nM) caused the highest induction of SMN2FL (3.8-fold) (FIG. 10D-10F).

Together, the data presented in FIG. 10 demonstrates that saRNA DS06-0004 alone has strong activity in inducing SMN2 mRNA expression especially SMN2FL in type II SMA cells. When the SMN2 saRNA was combined with Risdiplam, the greatest observed increase in SMN2FL could be achieved.

### 7.9. Example 9: Combinatory Effect of saRNA (DS06-0004) and Risdiplam on SMN Protein Levels in Type II SMA GM03813 Cells

To further verify the effect of Risdiplam and DS06-0004 alone or in combination on SMN2 gene expression in type II SMA cells GM03813, western blotting assays were performed in GM03813 cells treated with Risdiplam and DS06-0004 alone or in combination. As shown in FIGS. 11A and 11B, Risdiplam at 50 nM and 250 nM caused a 1.1- and 1.7-fold increase in SMN protein, respectively.

Treating cells with 50 nM of Risdiplam in combination with increasing concentrations of DS06-0004 (1 nM, 5 nM and 25 nM) induced SMN protein by 1.3-, 1.4-, and 1.8-fold, respectively (FIGS. 11A and 11B).

Further treating cells with 250 nM of Risdiplam in combination with increasing concentrations of DS06-0004 (1 nM, 5 nM and 25 nM) induced SMN protein by 2.1-, 2.3-, and 2.0-fold, respectively (FIGS. 11A and 11B).

Together, this data confirms that the combinatory effect of saRNA and Risdiplam in inducing SMN2 expression could be verified at the protein level.

### 7.10. Example 10: Combinatory Effect of saRNAs (DS06-0031 and DS06-0067) and ASO-10-27 on the Expression of Full-Length and Exon 7 Skipped SMN2 mRNA in GM03813 Cells

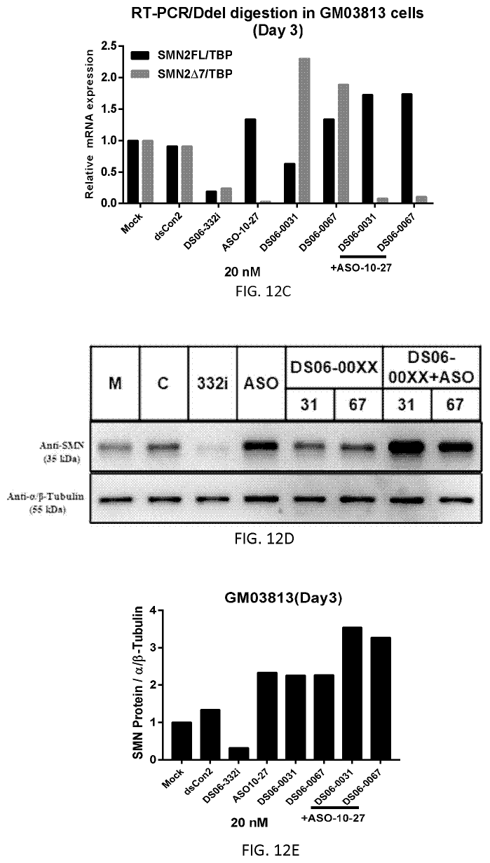

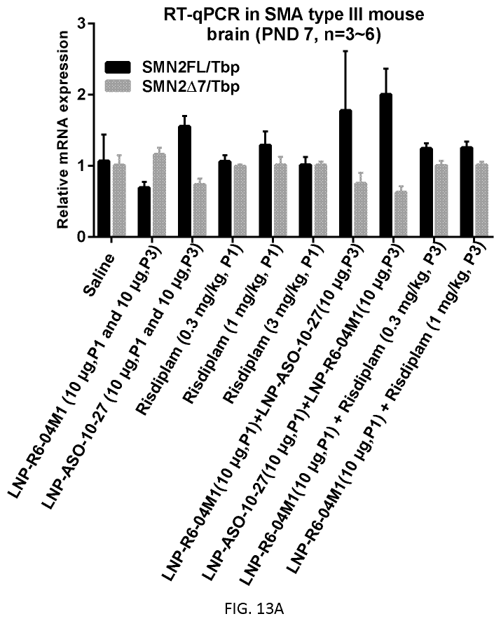

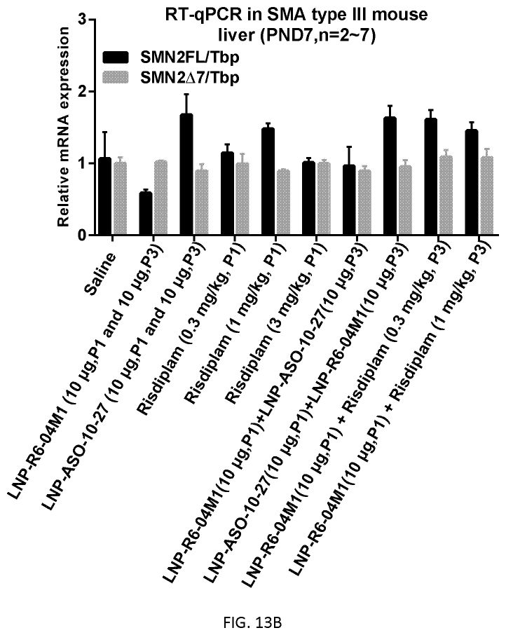

To determine the combinatory effects of ASO-10-27 with two saRNAs (DS06-0031 and DS06-0067) which induced more SMN2 Δ7 than SMN2FL mRNA expression in type II SMA cells putatively due to a transcription-coupled splicing modulation mechanism (FIGS. 12A and 12B), GM03813 cells were transfected with DS06-0031 or DS06-0067 and ASO-10-27 alone or in combination at 10 nM for 72 hours. SMN2 expression was assessed in the treated cells by RT-qPCR (FIG. 12A) and semi-quantitative RT-PCR (FIGS. 12B and 12C). As shown in FIG. 12A, DS06-0031 and DS06-0067 at 10 nM changed SMN2FL by 0.9- and 1.3-fold respectively and SMN2 Δ7 by 1.9- and 2.4-fold, respectively, whereas ASO-10-27 changed SMN2FL and SMN2 Δ7 by 1.4-fold and 0.3-fold, respectively.

When DS06-0031 or DS06-0067 was combined with ASO-10-27 at 10 nM in cell transfection, SMN2FL was induced by 2.0- and 3.2-fold and SMN2 Δ7 was decreased by 0.3- and 0.05-fold, respectively.

The RT-qPCR result presented in FIG. 12A was further verified by semi-quantitative RT-PCR. Consistent with RT-qPCR result, ASO-10-27 alone caused a 1.4-fold increase in SMN2FL mRNA at 10 nM and combination with DS06-0031 and DS06-0067 caused a 1.8- and 2.3-fold increase of SMN2FL and a concurrent reduction in SMN2 Δ7 by 0.3- and 0.02-fold (FIGS. 12B and 12C).

Furthermore, SMN protein levels were assessed by western blotting assay. Consistent with SMN2FL expression, in the presence of DS06-0031 and DS06-0067, ASO-10-27 caused a 3.6- and 3.3-fold increase in SMN protein levels compared to a 2.3-fold increase when it was used alone (FIGS. 12D and 12E).

Together, the data presented in FIG. 12 demonstrate that saRNAs (DS06-0031 and DS06-0067) alone had strong activity in inducing SMN2 expression, especially SMNΔ7. When they were combined with ASO-10-27, maximum induction of SMN2FL and reduction of SMN2 Δ7 could be achieved. This data also suggests that although the transcriptional activation and subsequent increase in pre-mRNA induced by certain saRNAs is reflected mainly by an increase in SMN2 Δ7, the increase in pre-mRNA supplies SMN2 mRNA modulators, e.g., ASOs, with additional substrates for exon 7 inclusion, leading to much enhanced SMN2FL mRNA and protein expression.

### 7.11. Example 11: Combinatory Effect of saRNAs (LNP-R6-04M1) with LNP-ASO-10-27 or Risdiplam on the Expression of SMN2FL and SMN2 Δ7 in SMA Type III Mice

The combinatory effect of LNP-R6-04M1 with LNP-ASO-10-27 or with Risdiplam were in evaluated in vivo, in SMA type IU mice. Neonatal mice were divided into 10 treatment groups:
Treatment Group 1: administration of LNP-R6-04M1 via ICV injection (injected twice on P1 and P3 respectively, each with 10 ug of LNP-R6-04M1);
Treatment Group 2: administration of LNP-ASO-10-27 via ICV injection (injected twice on P1 and P3 respectively, each with 10 ug of LNP-ASO-10-27);
Treatment Group 3: administration of Risdiplam via IP injection on P1 at a concentration of 0.3 mg/kg;
Treatment Group 4: administration of Risdiplam via IP injection on P1 at a concentration of 1 mg/kg;
Treatment Group 5: administration of Risdiplam via IP injection on P1 at a concentration of 3 mg/kg;
Treatment Group 6: combinatory treatment via administration of LNP-R6-04M1 and LNP-ASO-10-27. LNP-R6-04M1 was administered via ICV injection on P1 (10 ug), while LNP-ASO-10-27 was administered via ICV injection on P3 (10 ug);
Treatment Group 7: combinatory treatment via administration of LNP-ASO-10-27 and LNP-R6-04M1. LNP-ASO-10-27 was administered via ICV injection on P1 (10 ug), while LNP-R6-04M1 was administered via ICV injection on P3 (10 ug);
Treatment Group 8: combinatory treatment of LNP-R6-04M1 and Risdiplam. LNP-R6-04M1 was injected on P1 (10 ug) via ICV injection, and Risdiplam was injected via IP injection on P3 at a concentration of 0.3 mg/kg;
Treatment Group 9: combinatory treatment of LNP-R6-04M1 and Risdiplam. LNP-R6-04M1 was injected on P1 (10 ug) via ICV injection, and Risdiplam was injected via IP injection on P3 at a concentration of 1 mg/kg;

The SMA type LII mice were treated saline twice on P1 (5 μL) and P3(5 μL) via subcutaneous (SC) injection. P1 and P3 means postnatal day 1 and day3;

Treatment Group 10: treatment of mice with saline SMN2FL and SMN2 Δ7 mRNA levels were quantified by RT-qPCR in tissues of the brain, liver and spinal cord.

P1 and P3 means postnatal day 1 and day 3.Results

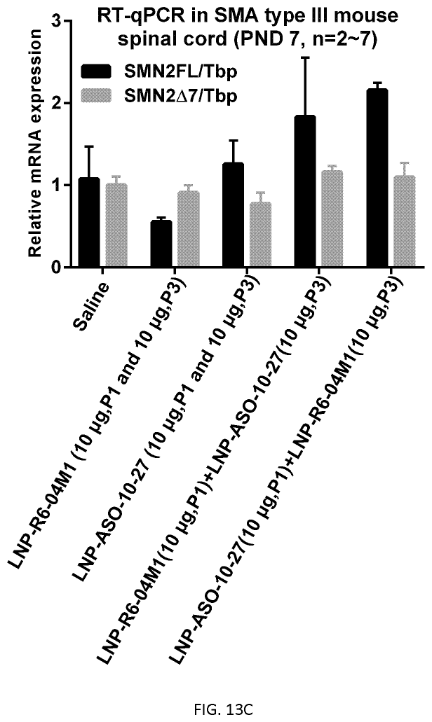

As shown in FIG. 13A, LNP-R6-04M1 (Treatment Group 1) induced an increase on SMN2 Δ7 mRNA expression in the brain by 1.2 fold relative to the control (Treatment Group 10), and didn't up-regulate the expression of SMN2FL mRNA in the brain. LNP-ASO-10-27 (Treatment Group 2) induced an increase on SMN2FL mRNA expression in the brain by 1.6 fold relative to the control (Treatment Group 10), and induced a decrease on SMN2 Δ7 mRNA expression in the brain by 0.7 fold. Risdipalm at concentrations of 0.3 mg/kg (Treatment Group 3), 1 mg/kg (Treatment Group 4) and 3 mg/kg (Treatment Group 5) all induced an increase in SMN2FL mRNA expression in the brain by 1.1, 1.3 and 1.0 fold relative to the control group and induced an increase in SMN2 Δ7 mRNA expression in the brain by 1.0 fold, respectively, relative to the control group (Treatment Group 10).

Combinatory treatment of LNP-R6-04M 1 on P1 (10 ug) and LNP-ASO-10-27 on P3 (10 ug) (Treatment Group 6), induced an increase in SMN2FL mRNA expression in the brain by 1.8 fold relative to the control group (Treatment Group 10) and decreased SMN2 Δ7 mRNA expression in the brain by 0.8 fold relative to the control group (Treatment Group 10).

Combinatory treatment of LNP-ASO-10-27 on P1 (10 ug) and LNP-R6-04M1 on P3 (10 ug) (Treatment Group 7), induced an increase in SMN2FL mRNA expression in the brain by 2.0 fold and decreased SMN2 Δ7 mRNA expression in the brain by 0.6 fold relative to the control group (Treatment Group 10).

Combinatory treatment of LNP-R6-04M 1 treated on P1(10 ug) and Risdiplam at a concentration of 0.3 mg/kg (Treatment Group 8) induced an increase in SMN2FL mRNA expression in the brain by 1.2 fold and SMN2 Δ7 mRNA expression in the brain by 1.0 fold, respectively, relative to the control group (Treatment Group 10).

Combinatory treatment of LNP-R6-04M1 treated on P1(10 ug) and Risdiplam at a concentration of 1 mg/kg (Treatment Group 9) induced an increase in SMN2FL mRNA expression in the brain by 1.3 fold and SMN2 Δ7 mRNA expression in the brain by 1.0 fold, respectively, relative to the control group (Treatment Group 10).

As shown in FIG. 13B, LNP-R6-04M1 (Treatment Group 1) didn't induce an increase on SMN2FL mRNA expression in the liver. LNP-ASO-10-27 (Treatment Group 2) induced an increase on SMN2FL mRNA expression in the liver by 1.7 fold relative to the control (Treatment Group 10), and induced decrease on SMN2 Δ7 mRNA expression in the liver by 0.9 fold. Risdipalm at concentrations of 0.3 mg/kg (Treatment Group 3), 1 mg/kg (Treatment Group 4) and 3 mg/kg (Treatment Group 5) all induced an increase in SMN2FL mRNA expression in the liver by 1.1, 1.5 and 1.0 fold, respectively, relative to the control group (Treatment Group 10) and induced an increase in SMN2 Δ7 mRNA expression in the liver by 1.0, 0.9, 1.0 fold, respectively, relative to the control group (Treatment Group 10).

Combinatory treatment of LNP-R6-04M1 on P1 (10 ug) and LNP-ASO-10-27 on P3 (10 ug) (Treatment Group 6), induced an increase in SMN2FL mRNA expression in the liver by 1.0 fold relative to the control group (Treatment Group 10) and decreased SMN2 Δ7 mRNA expression in the liver by 0.9 fold relative to the control group (Treatment Group 10).

Combinatory treatment of LNP-ASO-10-27 on P1 (10 ug) and LNP-R6-04M 1 on P3 (10 ug) (Treatment Group 7), induced an increase in SMN2FL mRNA expression in the liver by 1.6 fold and SMN2 Δ7 mRNA expression in the liver by 1.0 fold relative to the control group (Treatment Group 10).

Combinatory treatment of LNP-R6-04M1 treated on P1(10 ug) and Risdiplam at a concentration of 0.3 mg/kg (Treatment Group 8) induced an increase in SMN2FL mRNA expression in the liver by 1.6 fold and increased SMN2 Δ7 mRNA expression in the liver by 1.1 fold, respectively.

Combinatory treatment of LNP-R6-04M1 treated on P1(10 ug) and Risdiplam at a concentration of 1 mg/kg (Treatment Group 9) induced an increase in SMN2FL mRNA expression in the liver by 1.5 fold and increased SMN2 Δ7 mRNA expression in the liver by 1.1 fold, respectively.

As shown in FIG. 13C, LNP-R6-04M1 (Treatment Group 1) didn't induce increase on SMN2FL mRNA expression in the spinal cord. LNP-ASO-10-27 (Treatment Group 2) induced increase on SMN2FL mRNA expression in the spinal cord by 1.3 fold relative to the control, and induced decrease on SMN2 Δ7 mRNA expression in the spinal cord by 0.8 fold.

Combinatory treatment of LNP-R6-04M1 on P1 (10 ug) and LNP-ASO-10-27 on P3 (10 ug) (Treatment Group 6), induced an increase in SMN2FL mRNA expression in the spinal cord by 1.8 fold relative to the control group and increased SMN2 Δ7 mRNA expression in the spinal cord by 1.2 fold relative to the control group.

Combinatory treatment of LNP-ASO-10-27 on P1 (10 ug) and LNP-R6-04M1 on P3 (10 ug) (Treatment Group 7), induced an increase in SMN2FL mRNA expression in the spinal cord by 2.2 fold and increased SMN2 Δ7 mRNA expression in the spinal cord by 1.1 fold relative to the control group.

As shown in FIGS. 13A-13C, combinatory treatment with SMN2 saRNAs and SMN2 mRNA modulators results in an increase in SMN2FL mRNA expression and SMN2 Δ7 mRNA expression.

### 7.12. Materials and Methods

Oligonucleotide Design and Synthesis

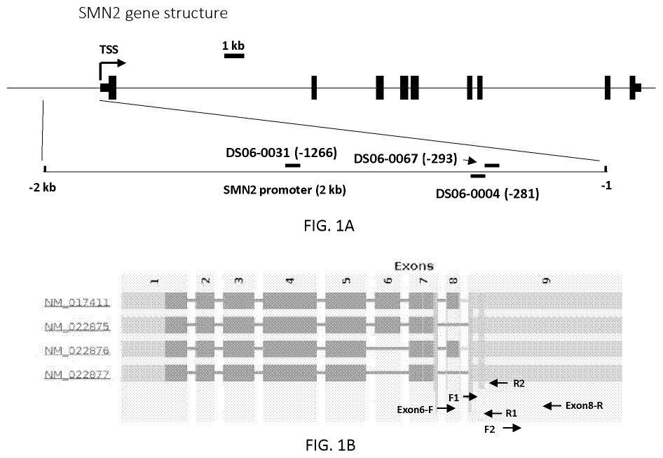

The saRNAs for SMN2 including DS06-0004 (also known as RAG6-281), DS06-0031 (also known as RAG6-1266) and DS06-0067 (also known as RAG6-293) were designed to target the SMN2 gene promoter at the −281, −1266 and −293 locations, respectively, relative to the transcription start site of SMN2 (FIG. 1). The SMN2 saRNAs were synthesized on a K&A DNA synthesizer (K&A Laborgeraete GbR, Schaaflieim, Germany) by using solid phase technique. Briefly, phosphoramidite monomers are added sequentially onto a solid support to generate the desired full-length oligonucleotide. Each cycle of base addition includes four chemical reactions, detritylation, coupling, oxidation/thiolation and capping. After synthesis, the solid support was then transferred to a screw-cap microcentrifuge tube. For a 1 μM synthesis scale, a mixture of 33% methylamine in ethanol and 1 ml of ammonium hydroxide was added. The tube containing the solid support was then heated in an oven at 60° C. to 65° C. for 2 hours and then allowed to cool to room temperature. The cleavage solution was collected and evaporated to dryness in a speedvac. The crude RNA oligonucleotide, still carrying the 2′-TBDMS groups, was dissolved in 0.1 ml of DMSO. After adding 1 ml of Triethylamine 3HF, the tube was capped, and the mixture was shaken vigorously to ensure complete dissolution. The bottle was heated in an oven at 60° C. to 65° C. for 3 to 3.5 hours. The tube was removed from the oven and cooled to room temperature. The solution containing the completely desilylated oligonucleotide was cooled on dry ice. 2 ml of ice-cold n-butanol (−20° C.) was carefully added in 0.5 ml portions to precipitate the oligonucleotide. The precipitate was filtered and washed with 1 ml ice-cold n-butanol and the precipitate was then dissolved in 1M TEAA (triethylammonium acetate). The crude oligonucleotides were then purified by exchange (IEX) HPLC using a source 15Q column. And the purity of the fractions was analyzed by ion exchange (IEX) HPLC using Column DNA Pac™ PA100. Following the generation of desalted purified single strand solutions, a duplex was made by annealing two complimentary single-stranded oligonucleotides and was lyophilized to powder.

ASO-10-27: Antisense oligonucleotide (ASO) ASO-10-27, also known as Nusinersen (Spinraza®), was synthesized using the same technique described above except the omission of the final annealing step. ASO-10-27 is a single stranded and 2′-O-2-methoxyethyl (MOE)-modified ASO and induces exon 7 inclusion by targeting an intronic splicing silencer (ISS) at intron 7 of SMN2 gene (Hua, Y, et al. Antisense masking of an hnRNP A1/A2 intronic splicing silencer corrects SMN2 splicing in transgenic mice.” Am J Human Genet (2008). The sequence for ASO-10-27 is: meU*meC*meA*meC*meU*meU*meU*meC*meA*meU*meA*meA*meU*meG*meC*meU*meG*meG, in which, me, 2′MOE, *, phosphorothioate (PS) backbone modification, all cytosines (Cs) are 5′methyl cytosine. Lyophilized oligonucleotides were suspended in RNase free water for cell transfection or diluted with saline to the appropriate concentration for in vivo injection.

Cell Culture and Treatment

SMA patient derived fibroblasts were obtained from Coriell Institute (Camden, N.J., USA), including GM00232 (SMA type I with 2 copies of SMN2 gene) and GM03813 (SMA type II with 3 copies of SMN2 gene). These cells were cultured at 5% C02 and 37° C. in modified MEM medium (Gibco, Thermo Fisher Scientific, Carlsbad, Calif.) supplemented with 15% bovine calf serum (Sigma-Aldrich), 1% NEAA (Gibco) and 1% penicillin/streptomycin (Gibco). To transfect oligonucleotides including saRNAs, siRNA and ASO, cells were seeded into 6-well plates at a density of 1×10⁵ cells/well and were transfected with oligonucleotides at different concentrations using RNAiMax (Invitrogen, Carlsbad, Calif.) according to the reverse transfection protocol provided by the manufacturer for 72 hours (unless otherwise specified). The sequences for saRNAs, an SMN2 siRNA (DS06-332i), a control dsRNA (dsCon2) and an ASO are listed in Table 1. For Risdiplam treatment of cells, Risdiplam (HY-109101, MedChem Express Company, Monmouth Junction, N.J., USA) dissolved in DMSO was added directly to cells at desired concentration for 72 hours unless specified otherwise.

TABLE 1
Oligonucleotide strand sequences and duplex compositions

| Title | Sequence No. | Sequence (5′-3′) | Length |
|---|---|---|---|
| DS06-0004 | SEQ ID NO: 476 | AGACGAGGCCUAAGCAACATT | 21 nt |
| | SEQ ID NO: 477 | UGUUGCUUAGGCCUCGUCUTT | 21 nt |
| DS06-0031 | SEQ ID NO: 478 | UUGUACACUUGGUCAACAUTT | 21 nt |
| | SEQ ID NO: 479 | AUGUUGACCAAGUGUACAATT | 21 nt |
| DS06-0067 | SEQ ID NO: 480 | CACUGGAGUUCGAGACGAGTT | 21 nt |
| | SEQ ID NO: 481 | CUCGUCUCGAACUCCAGUGTT | 21 nt |
| DS06-332i | SEQ ID NO: 482 | GGUGACAUUUGUGAAACUUTT | 21 nt |
| | SEQ ID NO: 483 | AAGUUUCACAAAUGUCACCTT | 21 nt |
| dsCon2 | SEQ ID NO: 484 | ACUACUGAGUGACAGUAGATT | 21 nt |
| | SEQ ID NO: 485 | UCUACUGUCACUCAGUAGUTT | 21 nt |
| ASO-10-27 | SEQ ID NO: 486 | meU*meC*meA*meC*meU*meU*meU*meC*me | 18 nt |
| | | A*meU*meA*meA*meU*meG*meC*meU*meG* | |
| | | meG | |
| LNP-R6-04M1 | SEQ ID NO: 488 | mG*fA*mCfGmAfGmGfCfCfUfAfAmGfCmAfA* | 18 nt |
| | SEQ ID NO: 496 | mC*fA | 20 nt |
| | | VpmU*fG*mUfUmGfCmUfUmAfGmGfCmC*fU* | |
| | | mC*fG*mU*fC*mU*fC | |
| LNP-ASO-10-27 | SEQ ID NO: 486 | meU*meC*meA*meC*meU*meU*meU*meC*me | 18 nt |
| | | A*meU*meA*meA*meU*meG*meC*meU*meG* | |
| | | meG | |

Note:
m, 2′-O-methyl (2′-OMe); me, 2′-O-methoxyethyl (2′MOE); f, 2′-fluoro; *, phosphorothioate (PS) backbone modification; underlined C, 5′ methyl cytosine; Vp, 5′-(E)-vinylphosphonate

RNA Isolation and Reverse Transcription-Quantitative Polymerase Chain Reaction (RT-QPCR)

For RNA isolation from cultured cells, total cellular RNA was isolated from treated cells using an RNeasy Plus Mini kit (Qiagen, Hilden, Germany) according to its manual. To isolated RNA from animal tissues, tissues were harvested and stored at RNA later (AM7021, Thermo Fisher). Total RNA was then isolated using MagPure Total RNA Micro LQ kit (Magen, R6621, China) by auto-pure96 machine (ALLSHENG, China). The resultant RNA (1 μg) was reverse transcribed into cDNA by using a PrimeScript RT kit containing gDNA Eraser (Takara, Shlga, Japan). The resultant cDNA was amplified in an ABI 7500 Fast Real-time PCR System (Applied Biosystems; Foster City, Calif.) using SYBR Premix Ex Taq 11 (Takara, Shlga, Japan) reagents and primers which specifically amplified full-length (SMN2FL) or A7 SMN2 mRNA (SMN2 Δ7) (FIG. 1). The reaction conditions were: 95° C. for 3 seconds (1 cycle) and 60° C. for 30 seconds (40 cycles). Amplification of TBP gene served as an internal control. All primer sequences are listed in Table 2. The RT and RT-qPCR reactions are shown in Table 3 and Table 4.

TABLE 2
Primer sequences for RT-qPCR assay

| Primer Title | Sequence No. | Sequence (5′-3′) | Product size |
|---|---|---|---|
| SMN2FL F | SEQ ID NO: 487 | TCATACTGGCTATTATATGGGTTTT | 95 bp |
| SMN2FL R | SEQ ID NO: 489 | TGCTCTATGCCAGCATTTCTC | |
| SMNΔ7 F | SEQ ID NO: 490 | GCTATTATATGGAAATGCTGGCATAG | 73 bp |
| SMNΔ7 R | SEQ ID NO: 491 | TTCCAGATCTGTCTGATCGTTTCT | |
| TBP F | SEQ ID NO: 492 | TGCTCACCCACCAACAATTTAG | 139 bp |
| TBP R | SEQ ID NO: 493 | TCTGCTCTGACTTTAGCACCTG | |
| Tbp F | SEQ ID NO: 497 | GCTCTGGAATTGTACCGCAG | 126 bp |
| Tbp R | SEQ ID NO: 498 | CTGCAGCAAATCGCTTGGGA | |

TABLE 3
RT reaction

| RT reaction-1 (Takara, RR047A) | Volume (μl) |
|---|---|
| 5 × gDNA Eraser Buffer | 2 |
| gDNA Eraser | 1 |
| Total RNA (1 μg) + RNase Free dH2O | 7 |
| Total | 10 |

42° C. 5 min, store at 4° C.

| Reagent-2 (Takara, RR047A) | Volume (μl) |
|---|---|
| 5 × PrimeScript Buffer2 | 4 |
| PrimeScript RT Enzyme Mix I | 1 |
| RT Prime Mix | 1 |
| RNase free dH2O | 4 |
| RT reaction-1 | 10 |
| Total | 20 |

37° C. 15 min, 85° C. 5 sec, store at 4° C.

TABLE 4
RT-qPCR reaction

| Reagent (Takara, RR820A) | Volume (μl) |
|---|---|
| SYBR Premix Ex Taq II (2×) | 5 |
| PCR Primer (F + R) 5 μM | 1 |
| cDNA (RT product) | 4 |
| Total | 10 |

Semi-Quantitative RT-PCR/DdeI Digestion Assay

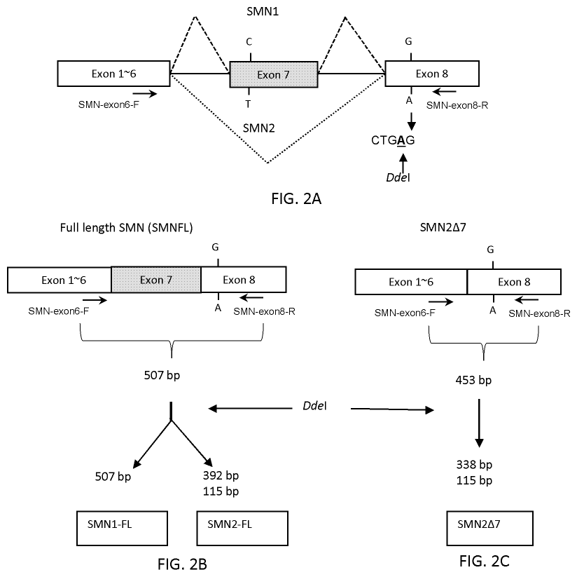

To amplify both SMN2FL and SMN2 Δ7 in one reaction, cDNA was amplified by semi-quantitative RT-PCR using primers that spans exon 7 of SMN2 (Table 5) (FIG. 2). The PCR reaction conditions were: 94° C. for 2 minutes (1 cycle), 98° C. for 10 seconds, 60° C. for 15 seconds, 72° C. for 32 seconds, cycled for 30 time with a final 5 minutes extension at 72° C. The PCR reaction is listed in Table 6. To further differentiate SMN1 mRNA from SMN2, the resultant PCR products of SMN were digested by DdeI restriction enzyme (R0175L, NEB) and then separated by 2% agarose gel. Due to a nucleotide variant on exon 8 of SMN2, a DdeI recognition site exists in PCR products amplified from SMN2 gene but not from SMN1 gene, DdeI digestion releases a 115 bp fragment from both SMN2FL and SMN2 Δ7, resulting 3 fragments: 507 (SMN1FL), 338 (SMN2 Δ7), 392 (SMN2FL) and 115 bp (FIG. 2). TBP gene was also amplified as a RNA loading control. The DdeI digestion reaction conditions were: 37° C. for 60 minutes and 65° C. for 20 minutes, 1cycle. The DdeI digestion reactions are listed in Table 7.

TABLE 6
Semi-quantitative RT-PCR reaction

| Reagents (Takara, R010A) | Volume (μl) |
|---|---|
| 5 × prime STAR Buffer | 5 |
| dNTP Mixture | 2 |
| Prime F + R (5 μM) | 1 |
| Template | 2 |
| PrimeSTAR HS DNA Polymerase | 0.25 |
| DD-Water | 14.75 |
| Total | 25 |

TABLE 5
Primer sequences for Semi-quantitative RT-PCR assay

| Primer Title | Sequence No. | Sequence (5′-3′) | Product size |
|---|---|---|---|
| SMN-exon6 F | SEQ ID NO: | CCCCCACCACCTCCCATATG | 507 bp and |
| SMN-exon8 R | 494 | CCCTTCTCACAGCTCATAAAATTAC | 453 bp |
| | SEQ ID NO: | | |
| | 495 | | |

TABLE 7
DdeI digestion reaction

| Reagents (NEB, R0175L) | Volume (μl) |
|---|---|
| Restriction Enzyme | 1 |
| 10 × NEB Buffer | 1 |
| cDNA | 6 |
| DD-Water | 2 |
| Total | 10 |

Western Blotting

Proteins were harvested from transfected cells using 1×RIPA Buffer including protease inhibitors and detected the protein concentration by BCA protein assay kits (Beyotime, P0010, China). To isolated proteins from animal tissues, tissues were harvested and lysed using 1×RIPA Buffer. Protein concentrations were measured by using BCA protein assay kits. Protein electrophoresis was performed (10 ug protein/well) with the use of a sodium dodecyl sulfate polyacrylamide gel electrophoresis (PAGE) gel, which was then transferred to a polyvinylidene difluoride (0.45 μm PVDF) membrane. The membranes were blotted with primary anti-SMN (CST, 19276, USA) or anti-α/β-Tubulin (CST, 2148s, USA) antibodies at 4° C. overnight. After three washes with TBST buffer, the membranes were incubated with anti-IgG, horseradish peroxidase-conjugated secondary antibodies (CST, 7074s and 7076s, USA) for 1 h at room temperature (RT). The membranes were then washed with TBST buffer three times for 10 min each and analyzed by Image Lab (BIO-RAD, Chemistry Doctm MP Imaging System). Band densities of SMN protein and α/β-Tubulin were quantified using ImageJ software.

Animal Procedures

All animal procedures were conducted by certified laboratory personnel using protocols consistent with local and state regulations and approved by the Institutional Animal Care and Use Committee. SMA-like mice created by homozygous knock-out of mouse Smn exon 7 with transgene of human SMN2 (Smn−/−SMN2+/−) as previously described Hsieh-Li et al. (Hsieh-Li, H. M., et al. A mouse model for spinal muscular atrophy. Nature Genet (2000) were obtained from Jackson Laboratories. Tail snips were gathered at postnatal day 0 (P0), and each pup was identified by paw tattooing and genotyped by PCR analysis using a set of 3 specific primers: S1, 5′-ATAACACCACCACTCTTACTC-3′, and S2, 5′-GTAGCCGTGATGCCATTGTCA-3′ (1,150 bp band for wild type alleles) and S1 and H1, 5′-AGCCTGAAGAACGAGATCAGC-3′ (950 bp band for mutant alleles). The PCR products were detected by 1% agarose gel. Severe SMA mice (Smn^{-/-}, SMN^{+/0}) were generated. Littermates heterozygous for mouse Smn (Smn1^{+/-}, SMN2^{+/-}) were used as controls.

Lipid Nanoparticle (LNP) Preparation

Lipid stock with DLin-KC2-DMA (50 mg/mL), cholesterol (10 mg/mL), DSPC (7.5 mg/mL) and PEG2000-DMPE (20 mg/mL) dissolved in 100% ethanol is mixed rapidly with oligonucleotide stock (20 mg/mL, 0.05 mM citrate buffer, pH 4.0) by microfluidic chip with 1:3 volume ratio at a flow rate of 12 mL/min. The molar ratio of DLin-KC2-DMA, cholesterol, DSPC and PEG2000-DMPE is 50:38.5:10:1.5. Then, this pre-formed vesicle was dialyzed using a dialysis tube in 1×PBS (pH 7.4) for 12 hours. The particle size of LNP was tested by dynamic light scattering using Brookhaven NanoBrook 90Plus Zeta. The RNA concentration is tested by A260 using NanoPhotometer N50.

Intracerebral Ventricle (ICV) Injection

Tail snips were gathered at postnatal day 0 (PND0) for genotyping by PCR and grouped as Type I SMA mice (Smn−/−, SMN2+/−), Type III SMA mice (Smn−/−, SMN2+/+), and heterozygous (Het) controls (Smn1+/−, SMN2+/−). Bilateral intracerebral ventricle (ICV) injection was performed under anesthesia via 2% isoflurane at a depth of 1.5 mm or 3.6 mm with 29-gauge syringe for pup (2 μL for each side, 5 mg/ml) on P1 and P3, respectively. Intraperitoneal (IP) injections were placed lower abdomen area of neonatal mice. The sequences for saRNA (LNP-R6-04M1) and LNP-ASO-10-27 are listed in Table 1.

TABLE 8
Sequences of SMN2 promoter-targeting saRNAs and their target DNA sequences

| | | | |
|---|---|---|---|
| RAG6-1763 | ATCTGTGAGATGTACCTTT | AUCUGUGAGAUGUACCUUU[dT][dT] | AAAGGUACAUCUCACAGAU[dT][dT] |
| | (SEQ ID NO: 315) | (SEQ ID NO: 1) | (SEQ ID NO: 158) |
| RAG6-1634 | CACTCTGTCACTCAGGCTG | CACUCUGUCACUCAGGCUG[dT][dT] | CAGCCUGAGUGACAGAGUG[dT][dT] |
| | (SEQ ID NO: 316) | (SEQ ID NO: 2) | (SEQ ID NO: 159) |
| RAG6-1633 | ACTCTGTCACTCAGGCTGG | ACUCUGUCACUCAGGCUGG[dT][dT] | CCAGCCUGAGUGACAGAGU[dT][dT] |
| | (SEQ ID NO: 317) | (SEQ ID NO: 3) | (SEQ ID NO: 160) |
| RAG6-1631 | TCTGTCACTCAGGCTGGAG | UCUGUCACUCAGGCUGGAG[dT][dT] | CUCCAGCCUGAGUGACAGA[dT][dT] |
| | (SEQ ID NO: 318) | (SEQ ID NO: 4) | (SEQ ID NO: 161) |
| RAG6-1623 | TCAGGCTGGAGTGCAGTGG | UCAGGCUGGAGUGCAGUGG[dT][dT] | CCACUGCACUCCAGCCUGA[dT][dT] |
| | (SEQ ID NO: 319) | (SEQ ID NO: 5) | (SEQ ID NO: 162) |
| RAG6-1615 | GAGTGCAGTGGCGTGATCT | GAGUGCAGUGGCGUGAUCU[dT][dT] | AGAUCACGCCACUGCACUC[dT][dT] |
| | (SEQ ID NO: 320) | (SEQ ID NO: 6) | (SEQ ID NO: 163) |
| RAG6-1612 | TGCAGTGGCGTGATCTTGG | UGCAGUGGCGUGAUCUUGG[dT][dT] | CCAAGAUCACGCCACUGCA[dT][dT] |
| | (SEQ ID NO: 321) | (SEQ ID NO: 7) | (SEQ ID NO: 164) |
| RAG6-1611 | GCAGTGGCGTGATCTTGGC | GCAGUGGCGUGAUCUUGGC[dT][dT] | GCCAAGAUCACGCCACUGC[dT][dT] |
| | (SEQ ID NO: 322) | (SEQ ID NO: 8) | (SEQ ID NO: 165) |
| RAG6-1606 | GGCGTGATCTTGGCTCACT | GGCGUGAUCUUGGCUCACU[dT][dT] | AGUGAGCCAAGAUCACGCC[dT][dT] |
| | (SEQ ID NO: 323) | (SEQ ID NO: 9) | (SEQ ID NO: 166) |
| RAG6-1603 | GTGATCTTGGCTCACTGCA | GUGAUCUUGGCUCACUGCA[dT][dT] | UGCAGUGAGCCAAGAUCAC[dT][dT] |
| | (SEQ ID NO: 324) | (SEQ ID NO: 10) | (SEQ ID NO: 167) |
| RAG6-1602 | TGATCTTGGCTCACTGCAA | UGAUCUUGGCUCACUGCAA[dT][dT] | UUGCAGUGAGCCAAGAUCA[dT][dT] |
| | (SEQ ID NO: 325) | (SEQ ID NO: 11) | (SEQ ID NO: 168) |
| RAG6-1600 | ATCTTGGCTCACTGCAACC | AUCUUGGCUCACUGCAACC[dT][dT] | GGUUGCAGUGAGCCAAGAU[dT][dT] |
| | (SEQ ID NO: 326) | (SEQ ID NO: 12) | (SEQ ID NO: 169) |
| RAG6-1598 | CTTGGCTCACTGCAACCTC | CUUGGCUCACUGCAACCUC[dT][dT] | GAGGUUGCAGUGAGCCAAG[dT][dT] |
| | (SEQ ID NO: 327) | (SEQ ID NO: 13) | (SEQ ID NO: 170) |
| RAG6-1597 | TTGGCTCACTGCAACCTCC | UUGGCUCACUGCAACCUCC[dT][dT] | GGAGGUUGCAGUGAGCCAA[dT][dT] |
| | (SEQ ID NO: 328) | (SEQ ID NO: 14) | (SEQ ID NO: 171) |
| RAG6-1578 | GCCTCCCGAGTTCAAGTGA | GCCUCCCGAGUUCAAGUGA[dT][dT] | UCACUUGAACUCGGGAGGC[dT][dT] |
| | (SEQ ID NO: 329) | (SEQ ID NO: 15) | (SEQ ID NO: 172) |
| RAG6-1577 | CCTCCCGAGTTCAAGTGAT | CCUCCCGAGUUCAAGUGAU[dT][dT] | AUCACUUGAACUCGGGAGG[dT][dT] |
| | (SEQ ID NO: 330) | (SEQ ID NO: 16) | (SEQ ID NO: 173) |
| RAG6-1576 | CTCCCGAGTTCAAGTGATT | CUCCCGAGUUCAAGUGAUU[dT][dT] | AAUCACUUGAACUCGGGAG[dT][dT] |
| | (SEQ ID NO: 331) | (SEQ ID NO: 17) | (SEQ ID NO: 174) |
| RAG6-1575 | TCCCGAGTTCAAGTGATTC | UCCCGAGUUCAAGUGAUUC[dT][dT] | GAAUCACUUGAACUCGGGA[dT][dT] |
| | (SEQ ID NO: 332) | (SEQ ID NO: 18) | (SEQ ID NO: 175) |
| RAG6-1567 | TCAAGTGATTCTCCTGGCT | UCAAGUGAUUCUCCUGGCU[dT][dT] | AGCCAGGAGAAUCACUUGA[dT][dT] |
| | (SEQ ID NO: 333) | (SEQ ID NO: 19) | (SEQ ID NO: 176) |
| RAG6-1565 | AAGTGATTCTCCTGGCTCA | AAGUGAUUCUCCUGGCUCA[dT][dT] | UGAGCCAGGAGAAUCACUU[dT][dT] |
| | (SEQ ID NO: 334) | (SEQ ID NO: 20) | (SEQ ID NO: 177) |
| RAG6-1564 | AGTGATTCTCCTGGCTCAG | AGUGAUUCUCCUGGCUCAG[dT][dT] | CUGAGCCAGGAGAAUCACU[dT][dT] |
| | (SEQ ID NO: 335) | (SEQ ID NO: 21) | (SEQ ID NO: 178) |
| RAG6-1563 | GTGATTCTCCTGGCTCAGC | GUGAUUCUCCUGGCUCAGC[dT][dT] | GCUGAGCCAGGAGAAUCAC[dT][dT] |
| | (SEQ ID NO: 336) | (SEQ ID NO: 22) | (SEQ ID NO: 179) |
| RAG6-1548 | CAGCCTCCCAAGCAGCTGT | CAGCCUCCCAAGCAGCUGU[dT][dT] | ACAGCUGCUUGGGAGGCUG[dT][dT] |
| | (SEQ ID NO: 337) | (SEQ ID NO: 23) | (SEQ ID NO: 180) |
| RAG6-1545 | CCTCCCAAGCAGCTGTCAT | CCUCCCAAGCAGCUGUCAU[dT][dT] | AUGACAGCUGCUUGGGAGG[dT][dT] |
| | (SEQ ID NO: 338) | (SEQ ID NO: 24) | (SEQ ID NO: 181) |
| RAG6-1543 | TCCCAAGCAGCTGTCATTA | UCCCAAGCAGCUGUCAUUA[dT][dT] | UAAUGACAGCUGCUUGGGA[dT][dT] |
| | (SEQ ID NO: 339) | (SEQ ID NO: 25) | (SEQ ID NO: 182) |
| RAG6-1535 | AGCTGTCATTACAGGCCTG | AGCUGUCAUUACAGGCCUG[dT][dT] | CAGGCCUGUAAUGACAGCU[dT][dT] |
| | (SEQ ID NO: 340) | (SEQ ID NO: 26) | (SEQ ID NO: 183) |
| RAG6-1534 | GCTGTCATTACAGGCCTGC | GCUGUCAUUACAGGCCUGC[dT][dT] | GCAGGCCUGUAAUGACAGC[dT][dT] |
| | (SEQ ID NO: 341) | (SEQ ID NO: 27) | (SEQ ID NO: 184) |
| RAG6-1533 | CTGTCATTACAGGCCTGCA | CUGUCAUUACAGGCCUGCA[dT][dT] | UGCAGGCCUGUAAUGACAG[dT][dT] |
| | (SEQ ID NO: 342) | (SEQ ID NO: 28) | (SEQ ID NO: 185) |
| RAG6-1483 | GGAGAAACAGGGTTTCACC | GGAGAAACAGGGUUUCACC[dT][dT] | GGUGAAACCCUGUUUCUCC[dT][dT] |
| | (SEQ ID NO: 343) | (SEQ ID NO: 29) | (SEQ ID NO: 186) |
| RAG6-1481 | AGAAACAGGGTTTCACCAT | AGAAACAGGGUUUCACCAU[dT][dT] | AUGGUGAAACCCUGUUUCU[dT][dT] |
| | (SEQ ID NO: 344) | (SEQ ID NO: 30) | (SEQ ID NO: 187) |
| RAG6-1403 | AAGTGCTGGGATTATAGGC | AAGUGCUGGGAUUAUAGGC[dT][dT] | GCCUAUAAUCCCAGCACUU[dT][dT] |
| | (SEQ ID NO: 345) | (SEQ ID NO: 31) | (SEQ ID NO: 188) |
| RAG6-1392 | TTATAGGCATGAGCCACCG | UUAUAGGCAUGAGCCACCG[dT][dT] | CGGUGGCUCAUGCCUAUAA[dT][dT] |
| | (SEQ ID NO: 346) | (SEQ ID NO: 32) | (SEQ ID NO: 189) |
| RAG6-1241 | ATTCTCCCCTTCCTCCACA | AUUCUCCCCUUCCUCCACA[dT][dT] | UGUGGAGGAAGGGGAGAAU[dT][dT] |
| | (SEQ ID NO: 347) | (SEQ ID NO: 33) | (SEQ ID NO: 190) |
| RAG6-1239 | TCTCCCCTTCCTCCACAGA | UCUCCCCUUCCUCCACAGA[dT][dT] | UCUGUGGAGGAAGGGGAGA[dT][dT] |
| | (SEQ ID NO: 348) | (SEQ ID NO: 34) | (SEQ ID NO: 191) |
| RAG6-1119 | CATTTAGCAACCCTAGATG | CAUUUAGCAACCCUAGAUG[dT][dT] | CAUCUAGGGUUGCUAAAUG[dT][dT] |
| | (SEQ ID NO: 349) | (SEQ ID NO: 35) | (SEQ ID NO: 192) |
| RAG6-1118 | ATTTAGCAACCCTAGATGC | AUUUAGCAACCCUAGAUGC[dT][dT] | GCAUCUAGGGUUGCUAAAU[dT][dT] |
| | (SEQ ID NO: 350) | (SEQ ID NO: 36) | (SEQ ID NO: 193) |
| RAG6-1117 | TTTAGCAACCCTAGATGCT | UUUAGCAACCCUAGAUGCU[dT][dT] | AGCAUCUAGGGUUGCUAAA[dT][dT] |
| | (SEQ ID NO: 351) | (SEQ ID NO: 37) | (SEQ ID NO: 194) |
| RAG6-1116 | TTAGCAACCCTAGATGCTT | UUAGCAACCCUAGAUGCUU[dT][dT] | AAGCAUCUAGGGUUGCUAA[dT][dT] |
| | (SEQ ID NO: 352) | (SEQ ID NO: 38) | (SEQ ID NO: 195) |
| RAG6-1115 | TAGCAACCCTAGATGCTTA | UAGCAACCCUAGAUGCUUA[dT][dT] | UAAGCAUCUAGGGUUGCUA[dT][dT] |
| | (SEQ ID NO: 353) | (SEQ ID NO: 39) | (SEQ ID NO: 196) |
| RAG6-1089 | ATACTGGAGGCCCGGTGTG | AUACUGGAGGCCCGGUGUG[dT][dT] | CACACCGGGCCUCCAGUAU[dT][dT] |
| | (SEQ ID NO: 354) | (SEQ ID NO: 40) | (SEQ ID NO: 197) |
| RAG6-1075 | GTGTGGTGGCTCACACCTG | GUGUGGUGGCUCACACCUG[dT][dT] | CAGGUGUGAGCCACCACAC[dT][dT] |
| | (SEQ ID NO: 355) | (SEQ ID NO: 41) | (SEQ ID NO: 198) |
| RAG6-1072 | TGGTGGCTCACACCTGTAA | UGGUGGCUCACACCUGUAA[dT][dT] | UUACAGGUGUGAGCCACCA[dT][dT] |
| | (SEQ ID NO: 356) | (SEQ ID NO: 42) | (SEQ ID NO: 199) |
| RAG6-1071 | GGTGGCTCACACCTGTAAT | GGUGGCUCACACCUGUAAU[dT][dT] | AUUACAGGUGUGAGCCACC[dT][dT] |
| | (SEQ ID NO: 357) | (SEQ ID NO: 43) | (SEQ ID NO: 200) |
| RAG6-1070 | GTGGCTCACACCTGTAATC | GUGGCUCACACCUGUAAUC[dT][dT] | GAUUACAGGUGUGAGCCAC[dT][dT] |
| | (SEQ ID NO: 358) | (SEQ ID NO: 44) | (SEQ ID NO: 201) |
| RAG6-1068 | GGCTCACACCTGTAATCCC | GGCUCACACCUGUAAUCCC[dT][dT] | GGGAUUACAGGUGUGAGCC[dT][dT] |
| | (SEQ ID NO: 359) | (SEQ ID NO: 45) | (SEQ ID NO: 202) |
| RAG6-1064 | CACACCTGTAATCCCAGCA | CACACCUGUAAUCCCAGCA[dT][dT] | UGCUGGGAUUACAGGUGUG[dT][dT] |
| | (SEQ ID NO: 360) | (SEQ ID NO: 46) | (SEQ ID NO: 203) |
| RAG6-1063 | ACACCTGTAATCCCAGCAC | ACACCUGUAAUCCCAGCAC[dT][dT] | GUGCUGGGAUUACAGGUGU[dT][dT] |
| | (SEQ ID NO: 361) | (SEQ ID NO: 47) | (SEQ ID NO: 204) |
| RAG6-1061 | ACCTGTAATCCCAGCACTT | ACCUGUAAUCCCAGCACUU[dT][dT] | AAGUGCUGGGAUUACAGGU[dT][dT] |
| | (SEQ ID NO: 362) | (SEQ ID NO: 48) | (SEQ ID NO: 205) |
| RAG6-1057 | GTAATCCCAGCACTTTGGG | GUAAUCCCAGCACUUUGGG[dT][dT] | CCCAAAGUGCUGGGAUUAC[dT][dT] |
| | (SEQ ID NO: 363) | (SEQ ID NO: 49) | (SEQ ID NO: 206) |
| RAG6-1056 | TAATCCCAGCACTTTGGGA | UAAUCCCAGCACUUUGGGA[dT][dT] | UCCCAAAGUGCUGGGAUUA[dT][dT] |
| | (SEQ ID NO: 364) | (SEQ ID NO: 50) | (SEQ ID NO: 207) |
| RAG6-1055 | AATCCCAGCACTTTGGGAG | AAUCCCAGCACUUUGGGAG[dT][dT] | CUCCCAAAGUGCUGGGAUU[dT][dT] |
| | (SEQ ID NO: 365) | (SEQ ID NO: 51) | (SEQ ID NO: 208) |
| RAG6-1050 | CAGCACTTTGGGAGGCCGA | CAGCACUUUGGGAGGCCGA[dT][dT] | UCGGCCUCCCAAAGUGCUG[dT][dT] |
| | (SEQ ID NO: 366) | (SEQ ID NO: 52) | (SEQ ID NO: 209) |
| RAG6-1033 | GAGGCGGTCGGATTACGAG | GAGGCGGUCGGAUUACGAG[dT][dT] | CUCGUAAUCCGACCGCCUC[dT][dT] |
| | (SEQ ID NO: 367) | (SEQ ID NO: 53) | (SEQ ID NO: 210) |
| RAG6-1031 | GGCGGTCGGATTACGAGGT | GGCGGUCGGAUUACGAGGU[dT][dT] | ACCUCGUAAUCCGACCGCC[dT][dT] |
| | (SEQ ID NO: 368) | (SEQ ID NO: 54) | (SEQ ID NO: 211) |
| RAG6-1030 | GCGGTCGGATTACGAGGTC | GCGGUCGGAUUACGAGGUC[dT][dT] | GACCUCGUAAUCCGACCGC[dT][dT] |
| | (SEQ ID NO: 369) | (SEQ ID NO: 55) | (SEQ ID NO: 212) |
| RAG6-1029 | CGGTCGGATTACGAGGTCA | CGGUCGGAUUACGAGGUCA[dT][dT] | UGACCUCGUAAUCCGACCG[dT][dT] |
| | (SEQ ID NO: 370) | (SEQ ID NO: 56) | (SEQ ID NO: 213) |
| RAG6-1028 | GGTCGGATTACGAGGTCAG | GGUCGGAUUACGAGGUCAG[dT][dT] | CUGACCUCGUAAUCCGACC[AT][dT] |
| | (SEQ ID NO: 371) | (SEQ ID NO: 57) | (SEQ ID NO: 214) |
| RAG6-1027 | GTCGGATTACGAGGTCAGG | GUCGGAUUACGAGGUCAGG[dT][dT] | CCUGACCUCGUAAUCCGAC[dT][dT] |
| | (SEQ ID NO: 372) | (SEQ ID NO: 58) | (SEQ ID NO: 215) |
| RAG6-1026 | TCGGATTACGAGGTCAGGA | UCGGAUUACGAGGUCAGGA[dT][dT] | UCCUGACCUCGUAAUCCGA[dT][dT] |
| | (SEQ ID NO: 373) | (SEQ ID NO: 59) | (SEQ ID NO: 216) |
| RAG6-1025 | CGGATTACGAGGTCAGGAG | CGGAUUACGAGGUCAGGAG[dT][dT] | CUCCUGACCUCGUAAUCCG[dT][dT] |
| | (SEQ ID NO: 374) | (SEQ ID NO: 60) | (SEQ ID NO: 217) |
| RAG6-1022 | ATTACGAGGTCAGGAGTTC | AUUACGAGGUCAGGAGUUC[dT][dT] | GAACUCCUGACCUCGUAAU[dT][dT] |
| | (SEQ ID NO: 375) | (SEQ ID NO: 61) | (SEQ ID NO: 218) |
| RAG6-1021 | TTACGAGGTCAGGAGTTCA | UUACGAGGUCAGGAGUUCA[dT][dT] | UGAACUCCUGACCUCGUAA[dT][dT] |
| | (SEQ ID NO: 376) | (SEQ ID NO: 62) | (SEQ ID NO: 219) |
| RAG6-1020 | TACGAGGTCAGGAGTTCAA | UACGAGGUCAGGAGUUCAA[dT][dT] | UUGAACUCCUGACCUCGUA[dT][dT] |
| | (SEQ ID NO: 377) | (SEQ ID NO: 63) | (SEQ ID NO: 220) |
| RAG6-1019 | ACGAGGTCAGGAGTTCAAG | ACGAGGUCAGGAGUUCAAG[dT][dT] | CUUGAACUCCUGACCUCGU[dT][dT] |
| | (SEQ ID NO: 378) | (SEQ ID NO: 64) | (SEQ ID NO: 221) |
| RAG6-1016 | AGGTCAGGAGTTCAAGACC | AGGUCAGGAGUUCAAGACC[dT][dT] | GGUCUUGAACUCCUGACCU[dT][dT] |
| | (SEQ ID NO: 379) | (SEQ ID NO: 65) | (SEQ ID NO: 222) |
| RAG6-1008 | AGTTCAAGACCAGCCTGGC | AGUUCAAGACCAGCCUGGC[dT][dT] | GCCAGGCUGGUCUUGAACU[dT][dT] |
| | (SEQ ID NO: 380) | (SEQ ID NO: 66) | (SEQ ID NO: 223) |
| RAG6-980 | GAAACCCCATCTTTACTAA | GAAACCCCAUCUUUACUAA[dT][dT] | UUAGUAAAGAUGGGGUUUC[dT][dT] |
| | (SEQ ID NO: 381) | (SEQ ID NO: 67) | (SEQ ID NO: 224) |
| RAG6-951 | ATTAGCCGGGTGTGGTGGT | AUUAGCCGGGUGUGGUGGU[dT][dT] | ACCACCACACCCGGCUAAU[dT][dT] |
| | (SEQ ID NO: 382) | (SEQ ID NO: 68) | (SEQ ID NO: 225) |
| RAG6-937 | GTGGTGGGCGCCTGTAATC | GUGGUGGGCGCCUGUAAUC[dT][dT] | GAUUACAGGCGCCCACCAC[dT][dT] |
| | (SEQ ID NO: 383) | (SEQ ID NO: 69) | (SEQ ID NO: 226) |
| RAG6-931 | GGCGCCTGTAATCCCAGCT | GGCGCCUGUAAUCCCAGCU[dT][dT] | AGCUGGGAUUACAGGCGCC[dT][dT] |
| | (SEQ ID NO: 384) | (SEQ ID NO: 70) | (SEQ ID NO: 227) |
| RAG6-923 | TAATCCCAGCTACTCGGGG | UAAUCCCAGCUACUCGGGG[dT][dT] | CCCCGAGUAGCUGGGAUUA[dT][dT] |
| | (SEQ ID NO: 385) | (SEQ ID NO: 71) | (SEQ ID NO: 228) |
| RAG6-905 | GGGCTGAGGCAGAATTGCT | GGGCUGAGGCAGAAUUGCU[dT][dT] | AGCAAUUCUGCCUCAGCCC[dT][dT] |
| | (SEQ ID NO: 386) | (SEQ ID NO: 72) | (SEQ ID NO: 229) |
| RAG6-898 | GGCAGAATTGCTTGAACCT | GGCAGAAUUGCUUGAACCU[dT][dT] | AGGUUCAAGCAAUUCUGCC[dT][dT] |
| | (SEQ ID NO: 387) | (SEQ ID NO: 73) | (SEQ ID NO: 230) |
| RAG6-896 | CAGAATTGCTTGAACCTGG | CAGAAUUGCUUGAACCUGG[dT][dT] | CCAGGUUCAAGCAAUUCUG[dT][dT] |
| | (SEQ ID NO: 388) | (SEQ ID NO: 74) | (SEQ ID NO: 231) |
| RAG6-886 | TGAACCTGGGAGGCAGAGG | UGAACCUGGGAGGCAGAGG[dT][dT] | CCUCUGCCUCCCAGGUUCA[dT][dT] |
| | (SEQ ID NO: 389) | (SEQ ID NO: 75) | (SEQ ID NO: 232) |
| RAG6-885 | GAACCTGGGAGGCAGAGGT | GAACCUGGGAGGCAGAGGU[dT][dT] | ACCUCUGCCUCCCAGGUUC[dT][dT] |
| | (SEQ ID NO: 390) | (SEQ ID NO: 76) | (SEQ ID NO: 233) |
| RAG6-883 | ACCTGGGAGGCAGAGGTTG | ACCUGGGAGGCAGAGGUUG[dT][dT] | CAACCUCUGCCUCCCAGGU[dT][dT] |
| | (SEQ ID NO: 391) | (SEQ ID NO: 77) | (SEQ ID NO: 234) |
| RAG6-866 | TGCAGTGAGCTGAGATCAC | UGCAGUGAGCUGAGAUCAC[dT][dT] | GUGAUCUCAGCUCACUGCA[dT][dT] |
| | (SEQ ID NO: 392) | (SEQ ID NO: 78) | (SEQ ID NO: 235) |
| RAG6-857 | CTGAGATCACGCCACTGCA | CUGAGAUCACGCCACUGCA[dT][dT] | UGCAGUGGCGUGAUCUCAG[dT][dT] |
| | (SEQ ID NO: 393) | (SEQ ID NO: 79) | (SEQ ID NO: 236) |
| RAG6-852 | ATCACGCCACTGCATTCCA | AUCACGCCACUGCAUUCCA[dT][dT] | UGGAAUGCAGUGGCGUGAU[dT][dT] |
| | (SEQ ID NO: 394) | (SEQ ID NO: 80) | (SEQ ID NO: 237) |
| RAG6-829 | GGGTGACAGAGCAATACTC | GGGUGACAGAGCAAUACUC[dT][dT] | GAGUAUUGCUCUGUCACCC[dT][dT] |
| | (SEQ ID NO: 395) | (SEQ ID NO: 81) | (SEQ ID NO: 238) |
| RAG6-826 | TGACAGAGCAATACTCTGT | UGACAGAGCAAUACUCUGU[dT][dT] | ACAGAGUAUUGCUCUGUCA[dT][dT] |
| | (SEQ ID NO: 396) | (SEQ ID NO: 82) | (SEQ ID NO: 239) |
| RAG6-822 | AGAGCAATACTCTGTCGCA | AGAGCAAUACUCUGUCGCA[dT][dT] | UGCGACAGAGUAUUGCUCU[dT][dT] |
| | (SEQ ID NO: 397) | (SEQ ID NO: 83) | (SEQ ID NO: 240) |
| RAG6-796 | AAAAGAATACTGGAGGCTG | AAAAGAAUACUGGAGGCUG[dT][AT] | CAGCCUCCAGUAUUCUUUU[AT][dT] |
| | (SEQ ID NO: 398) | (SEQ ID NO: 84) | (SEQ ID NO: 241) |
| RAG6-795 | AAAGAATACTGGAGGCTGG | AAAGAAUACUGGAGGCUGG[dT][dT] | CCAGCCUCCAGUAUUCUUU[dT][dT] |
| | (SEQ ID NO: 399) | (SEQ ID NO: 85) | (SEQ ID NO: 242) |
| RAG6-790 | ATACTGGAGGCTGGGCGAG | AUACUGGAGGCUGGGCGAG[dT][dT] | CUCGCCCAGCCUCCAGUAU[dT][dT] |
| | (SEQ ID NO: 400) | (SEQ ID NO: 86) | (SEQ ID NO: 243) |
| RAG6-775 | CGAGGTGGCTCACACCTGT | CGAGGUGGCUCACACCUGU[dT][dT] | ACAGGUGUGAGCCACCUCG[dT][dT] |
| | (SEQ ID NO: 401) | (SEQ ID NO: 87) | (SEQ ID NO: 244) |
| RAG6-772 | GGTGGCTCACACCTGTAAT | GGUGGCUCACACCUGUAAU[dT][dT] | AUUACAGGUGUGAGCCACC[dT][dT] |
| | (SEQ ID NO: 402) | (SEQ ID NO: 88) | (SEQ ID NO: 245) |
| RAG6-769 | GGCTCACACCTGTAATCCC | GGCUCACACCUGUAAUCCC[dT][dT] | GGGAUUACAGGUGUGAGCC[dT][dT] |
| | (SEQ ID NO: 403) | (SEQ ID NO: 89) | (SEQ ID NO: 246) |
| RAG6-768 | GCTCACACCTGTAATCCCA | GCUCACACCUGUAAUCCCA[dT][dT] | UGGGAUUACAGGUGUGAGC[dT][dT] |
| | (SEQ ID NO: 404) | (SEQ ID NO: 90) | (SEQ ID NO: 247) |
| RAG6-765 | CACACCTGTAATCCCAGCA | CACACCUGUAAUCCCAGCA[dT][dT] | UGCUGGGAUUACAGGUGUG[dT][dT] |
| | (SEQ ID NO: 405) | (SEQ ID NO: 91) | (SEQ ID NO: 248) |
| RAG6-757 | TAATCCCAGCATTTTGGGA | UAAUCCCAGCAUUUUGGGA[dT][dT] | UCCCAAAAUGCUGGGAUUA[dT][dT] |
| | (SEQ ID NO: 406) | (SEQ ID NO: 92) | (SEQ ID NO: 249) |
| RAG6-728 | GGGCGGAATATCTTGAGCT | GGGCGGAAUAUCUUGAGCU[dT][dT] | AGCUCAAGAUAUUCCGCCC[dT][dT] |
| | (SEQ ID NO: 407) | (SEQ ID NO: 93) | (SEQ ID NO: 250) |
| RAG6-722 | AATATCTTGAGCTCAGGAG | AAUAUCUUGAGCUCAGGAG[dT][dT] | CUCCUGAGCUCAAGAUAUU[dT][dT] |
| | (SEQ ID NO: 408) | (SEQ ID NO: 94) | (SEQ ID NO: 251) |
| RAG6-703 | TTCGAGACCAGCCTACACA | UUCGAGACCAGCCUACACA[dT][dT] | UGUGUAGGCUGGUCUCGAA[dT][dT] |
| | (SEQ ID NO: 409) | (SEQ ID NO: 95) | (SEQ ID NO: 252) |
| RAG6-696 | CCAGCCTACACAATATGCT | CCAGCCUACACAAUAUGCU[dT][dT] | AGCAUAUUGUGUAGGCUGG[dT][dT] |
| | (SEQ ID NO: 410) | (SEQ ID NO: 96) | (SEQ ID NO: 253) |
| RAG6-689 | ACACAATATGCTCCAAACG | ACACAAUAUGCUCCAAACG[dT][dT] | CGUUUGGAGCAUAUUGUGU[dT][dT] |
| | (SEQ ID NO: 411) | (SEQ ID NO: 97) | (SEQ ID NO: 254) |
| RAG6-688 | CACAATATGCTCCAAACGC | CACAAUAUGCUCCAAACGC[dT][dT] | GCGUUUGGAGCAUAUUGUG[dT][dT] |
| | (SEQ ID NO: 412) | (SEQ ID NO: 98) | (SEQ ID NO: 255) |
| RAG6-687 | ACAATATGCTCCAAACGCC | ACAAUAUGCUCCAAACGCC[dT][dT] | GGCGUUUGGAGCAUAUUGU[dT][dT] |
| | (SEQ ID NO: 413) | (SEQ ID NO: 99) | (SEQ ID NO: 256) |
| RAG6-676 | CAAACGCCGCCTCTACAAA | CAAACGCCGCCUCUACAAA[dT][dT] | UUUGUAGAGGCGGCGUUUG[dT][dT] |
| | (SEQ ID NO: 414) | (SEQ ID NO: 100) | (SEQ ID NO: 257) |
| RAG6-622 | CTGTGGTCCTAGCTACTTG | CUGUGGUCCUAGCUACUUG[dT][dT] | CAAGUAGCUAGGACCACAG[dT][dT] |
| | (SEQ ID NO: 415) | (SEQ ID NO: 101) | (SEQ ID NO: 258) |
| RAG6-591 | GGGAGGATCGCTTGAGCTC | GGGAGGAUCGCUUGAGCUC[dT][dT] | GAGCUCAAGCGAUCCUCCC[AT][dT] |
| | (SEQ ID NO: 416) | (SEQ ID NO: 102) | (SEQ ID NO: 259) |
| RAG6-589 | GAGGATCGCTTGAGCTCGG | GAGGAUCGCUUGAGCUCGG[dT][dT] | CCGAGCUCAAGCGAUCCUC[dT][dT] |
| | (SEQ ID NO: 417) | (SEQ ID NO: 103) | (SEQ ID NO: 260) |
| RAG6-571 | GGAGGTCGAGGCTGCAATG | GGAGGUCGAGGCUGCAAUG[dT][dT] | CAUUGCAGCCUCGACCUCC[dT][dT] |
| | (SEQ ID NO: 418) | (SEQ ID NO: 104) | (SEQ ID NO: 261) |
| RAG6-568 | GGTCGAGGCTGCAATGAGC | GGUCGAGGCUGCAAUGAGC[dT][dT] | GCUCAUUGCAGCCUCGACC[dT][dT] |
| | (SEQ ID NO: 419) | (SEQ ID NO: 105) | (SEQ ID NO: 262) |
| RAG6-557 | CAATGAGCCGAGATGGTGC | CAAUGAGCCGAGAUGGUGC[dT][dT] | GCACCAUCUCGGCUCAUUG[dT][dT] |
| | (SEQ ID NO: 420) | (SEQ ID NO: 106) | (SEQ ID NO: 263) |
| RAG6-556 | AATGAGCCGAGATGGTGCC | AAUGAGCCGAGAUGGUGCC[dT][dT] | GGCACCAUCUCGGCUCAUU[dT][dT] |
| | (SEQ ID NO: 421) | (SEQ ID NO: 107) | (SEQ ID NO: 264) |
| RAG6-550 | CCGAGATGGTGCCACTGCA | CCGAGAUGGUGCCACUGCA[dT][dT] | UGCAGUGGCACCAUCUCGG[dT][dT] |
| | (SEQ ID NO: 422) | (SEQ ID NO: 108) | (SEQ ID NO: 265) |
| RAG6-548 | GAGATGGTGCCACTGCACT | GAGAUGGUGCCACUGCACU[dT][dT] | AGUGCAGUGGCACCAUCUC[dT][dT] |
| | (SEQ ID NO: 423) | (SEQ ID NO: 109) | (SEQ ID NO: 266) |
| RAG6-547 | AGATGGTGCCACTGCACTC | AGAUGGUGCCACUGCACUC[dT][dT] | GAGUGCAGUGGCACCAUCU[dT][dT] |
| | (SEQ ID NO: 424) | (SEQ ID NO: 110) | (SEQ ID NO: 267) |
| RAG6-545 | ATGGTGCCACTGCACTCTG | AUGGUGCCACUGCACUCUG[dT][dT] | CAGAGUGCAGUGGCACCAU[dT][dT] |
| | (SEQ ID NO: 425) | (SEQ ID NO: 111) | (SEQ ID NO: 268) |
| RAG6-539 | CCACTGCACTCTGACGACA | CCACUGCACUCUGACGACA[dT][dT] | UGUCGUCAGAGUGCAGUGG[dT][dT] |
| | (SEQ ID NO: 426) | (SEQ ID NO: 112) | (SEQ ID NO: 269) |
| RAG6-538 | CACTGCACTCTGACGACAG | CACUGCACUCUGACGACAG[dT][dT] | CUGUCGUCAGAGUGCAGUG[dT][dT] |
| | (SEQ ID NO: 427) | (SEQ ID NO: 113) | (SEQ ID NO: 270) |
| RAG6-530 | TCTGACGACAGAGCGAGAC | UCUGACGACAGAGCGAGAC[dT][dT] | GUCUCGCUCUGUCGUCAGA[dT][dT] |
| | (SEQ ID NO: 428) | (SEQ ID NO: 114) | (SEQ ID NO: 271) |
| RAG6-529 | CTGACGACAGAGCGAGACT | CUGACGACAGAGCGAGACU[dT][dT] | AGUCUCGCUCUGUCGUCAG[dT][dT] |
| | (SEQ ID NO: 429) | (SEQ ID NO: 115) | (SEQ ID NO: 272) |
| RAG6-516 | GAGACTCCGTCTCAAAACA | GAGACUCCGUCUCAAAACA[dT][dT] | UGUUUUGAGACGGAGUCUC[dT][dT] |
| | (SEQ ID NO: 430) | (SEQ ID NO: 116) | (SEQ ID NO: 273) |
| RAG6-515 | AGACTCCGTCTCAAAACAA | AGACUCCGUCUCAAAACAA[dT][dT] | UUGUUUUGAGACGGAGUCU[dT][dT] |
| | (SEQ ID NO: 431) | (SEQ ID NO: 117) | (SEQ ID NO: 274) |
| RAG6-465 | TCTAGTGTTTAAGGATCTG | UCUAGUGUUUAAGGAUCUG[dT][dT] | CAGAUCCUUAAACACUAGA[dT][dT] |
| | (SEQ ID NO: 432) | (SEQ ID NO: 118) | (SEQ ID NO: 275) |
| RAG6-463 | TAGTGTTTAAGGATCTGCC | UAGUGUUUAAGGAUCUGCC[dT][dT] | GGCAGAUCCUUAAACACUA[dT][dT] |
| | (SEQ ID NO: 433) | (SEQ ID NO: 119) | (SEQ ID NO: 276) |
| RAG6-460 | TGTTTAAGGATCTGCCTTC | UGUUUAAGGAUCUGCCUUC[dT][dT] | GAAGGCAGAUCCUUAAACA[dT][dT] |
| | (SEQ ID NO: 434) | (SEQ ID NO: 120) | (SEQ ID NO: 277) |
| RAG6-453 | GGATCTGCCTTCCTTCCTG | GGAUCUGCCUUCCUUCCUG[dT][dT] | CAGGAAGGAAGGCAGAUCC[dT][dT] |
| | (SEQ ID NO: 435) | (SEQ ID NO: 121) | (SEQ ID NO: 278) |
| RAG6-425 | TTGTCTTTCCTTGTTTGTC | UUGUCUUUCCUUGUUUGUC[dT][dT] | GACAAACAAGGAAAGACAA[dT][dT] |
| | (SEQ ID NO: 436) | (SEQ ID NO: 122) | (SEQ ID NO: 279) |
| RAG6-423 | GTCTTTCCTTGTTTGTCTT | GUCUUUCCUUGUUUGUCUU[dT][dT] | AAGACAAACAAGGAAAGAC[dT][dT] |
| | (SEQ ID NO: 437) | (SEQ ID NO: 123) | (SEQ ID NO: 280) |
| RAG6-395 | CAAGCAGGTTTTAAATTCC | CAAGCAGGUUUUAAAUUCC[dT][dT] | GGAAUUUAAAACCUGCUUG[dT][dT] |
| | (SEQ ID NO: 438) | (SEQ ID NO: 124) | (SEQ ID NO: 281) |
| RAG6-392 | GCAGGTTTTAAATTCCTAG | GCAGGUUUUAAAUUCCUAG[dT][dT] | CUAGGAAUUUAAAACCUGC[dT][dT] |
| | (SEQ ID NO: 439) | (SEQ ID NO: 125) | (SEQ ID NO: 282) |
| RAG6-364 | ACATTTACTTTTCCAAGGG | ACAUUUACUUUUCCAAGGG[dT][dT] | CCCUUGGAAAAGUAAAUGU[dT][dT] |
| | (SEQ ID NO: 440) | (SEQ ID NO: 126) | (SEQ ID NO: 283) |
| RAG6-294 | ACACTGGAGTTCGAGACGA | ACACUGGAGUUCGAGACGA[dT][dT] | UCGUCUCGAACUCCAGUGU[dT][dT] |
| | (SEQ ID NO: 441) | (SEQ ID NO: 127) | (SEQ ID NO: 284) |
| RAG6-291 | CTGGAGTTCGAGACGAGGC | CUGGAGUUCGAGACGAGGC[dT][dT] | GCCUCGUCUCGAACUCCAG[dT][dT] |
| | (SEQ ID NO: 442) | (SEQ ID NO: 128) | (SEQ ID NO: 285) |
| RAG6-285 | TTCGAGACGAGGCCTAAGC | UUCGAGACGAGGCCUAAGC[dT][dT] | GCUUAGGCCUCGUCUCGAA[dT][dT] |
| | (SEQ ID NO: 443) | (SEQ ID NO: 129) | (SEQ ID NO: 286) |
| RAG6-282 | GAGACGAGGCCTAAGCAAC | GAGACGAGGCCUAAGCAAC[dT][dT] | GUUGCUUAGGCCUCGUCUC[dT][dT] |
| | (SEQ ID NO: 444) | (SEQ ID NO: 130) | (SEQ ID NO: 287) |
| RAG6-281 | AGACGAGGCCTAAGCAACA | AGACGAGGCCUAAGCAACA[dT][dT] | UGUUGCUUAGGCCUCGUCU[dT][dT] |
| | (SEQ ID NO: 445) | (SEQ ID NO: 131) | (SEQ ID NO: 288) |
| RAG6-280 | GACGAGGCCTAAGCAACAT | GACGAGGCCUAAGCAACAU[dT][dT] | AUGUUGCUUAGGCCUCGUC[dT][dT] |
| | (SEQ ID NO: 446) | (SEQ ID NO: 132) | (SEQ ID NO: 289) |
| RAG6-273 | CCTAAGCAACATGCCGAAA | CCUAAGCAACAUGCCGAAA[dT][dT] | UUUCGGCAUGUUGCUUAGG[dT][dT] |
| | (SEQ ID NO: 447) | (SEQ ID NO: 133) | (SEQ ID NO: 290) |
| RAG6-272 | CTAAGCAACATGCCGAAAC | CUAAGCAACAUGCCGAAAC[dT][dT] | GUUUCGGCAUGUUGCUUAG[dT][dT] |
| | (SEQ ID NO: 448) | (SEQ ID NO: 134) | (SEQ ID NO: 291) |
| RAG6-271 | TAAGCAACATGCCGAAACC | UAAGCAACAUGCCGAAACC[dT][dT] | GGUUUCGGCAUGUUGCUUA[dT][dT] |
| | (SEQ ID NO: 449) | (SEQ ID NO: 135) | (SEQ ID NO: 292) |
| RAG6-219 | TGGTGGCGCACGCCTATAG | UGGUGGCGCACGCCUAUAG[dT][dT] | CUAUAGGCGUGCGCCACCA[dT][dT] |
| | (SEQ ID NO: 450) | (SEQ ID NO: 136) | (SEQ ID NO: 293) |
| RAG6-218 | GGTGGCGCACGCCTATAGT | GGUGGCGCACGCCUAUAGU[dT][dT] | ACUAUAGGCGUGCGCCACC[dT][dT] |
| | (SEQ ID NO: 451) | (SEQ ID NO: 137) | (SEQ ID NO: 294) |
| RAG6-206 | CTATAGTCCTAGCTACTGG | CUAUAGUCCUAGCUACUGG[dT][dT] | CCAGUAGCUAGGACUAUAG[dT][dT] |
| | (SEQ ID NO: 452) | (SEQ ID NO: 138) | (SEQ ID NO: 295) |
| RAG6-205 | TATAGTCCTAGCTACTGGG | UAUAGUCCUAGCUACUGGG[dT][dT] | CCCAGUAGCUAGGACUAUA[dT][dT] |
| | (SEQ ID NO: 453) | (SEQ ID NO: 139) | (SEQ ID NO: 296) |
| RAG6-181 | TGAGGTGGGAGGATCGCTT | UGAGGUGGGAGGAUCGCUU[dT][dT] | AAGCGAUCCUCCCACCUCA[dT][dT] |
| | (SEQ ID NO: 454) | (SEQ ID NO: 140) | (SEQ ID NO: 297) |
| RAG6-144 | CTGCAGTGAGCCGAGATCG | CUGCAGUGAGCCGAGAUCG[dT][dT] | CGAUCUCGGCUCACUGCAG[dT][dT] |
| | (SEQ ID NO: 455) | (SEQ ID NO: 141) | (SEQ ID NO: 298) |
| RAG6-143 | TGCAGTGAGCCGAGATCGC | UGCAGUGAGCCGAGAUCGC[dT][dT] | GCGAUCUCGGCUCACUGCA[dT][dT] |
| | (SEQ ID NO: 456) | (SEQ ID NO: 142) | (SEQ ID NO: 299) |
| RAG6-119 | TGCACTCCAGCCTGAGCGA | UGCACUCCAGCCUGAGCGA[dT][dT] | UCGCUCAGGCUGGAGUGCA[dT][dT] |
| | (SEQ ID NO: 457) | (SEQ ID NO: 143) | (SEQ ID NO: 300) |
| RAG6-117 | CACTCCAGCCTGAGCGACA | CACUCCAGCCUGAGCGACA[dT][dT] | UGUCGCUCAGGCUGGAGUG[dT][dT] |
| | (SEQ ID NO: 458 | (SEQ ID NO: 144) | (SEQ ID NO: 301) |
| RAG6-101 | ACAGGGCGAGGCTCTGTCT | ACAGGGCGAGGCUCUGUCU[dT][dT] | AGACAGAGCCUCGCCCUGU[dT][dT] |
| | (SEQ ID NO: 459) | (SEQ ID NO: 145) | (SEQ ID NO: 302) |
| RAG6-98 | GGGCGAGGCTCTGTCTCAA | GGGCGAGGCUCUGUCUCAA[dT][dT] | UUGAGACAGAGCCUCGCCC[dT][dT] |
| | (SEQ ID NO: 460) | (SEQ ID NO: 146) | (SEQ ID NO: 303) |
| RAG6-97 | GGCGAGGCTCTGTCTCAAA | GGCGAGGCUCUGUCUCAAA[dT][dT] | UUUGAGACAGAGCCUCGCC[dT][dT] |
| | (SEQ ID NO: 461) | (SEQ ID NO: 147) | (SEQ ID NO: 304) |
| RAG6-96 | GCGAGGCTCTGTCTCAAAA | GCGAGGCUCUGUCUCAAAA[dT][dT] | UUUUGAGACAGAGCCUCGC[dT][dT] |
| | (SEQ ID NO: 462) | (SEQ ID NO: 148) | (SEQ ID NO: 305) |
| RAG6-93 | AGGCTCTGTCTCAAAACAA | AGGCUCUGUCUCAAAACAA[dT][dT] | UUGUUUUGAGACAGAGCCU[dT][dT] |
| | (SEQ ID NO: 463) | (SEQ ID NO: 149) | (SEQ ID NO: 306) |
| RAG6-92 | GGCTCTGTCTCAAAACAAA | GGCUCUGUCUCAAAACAAA[dT][dT] | UUUGUUUUGAGACAGAGCC[dT][dT] |
| | (SEQ ID NO: 464) | (SEQ ID NO: 150) | (SEQ ID NO: 307) |
| RAG6-91 | GCTCTGTCTCAAAACAAAC | GCUCUGUCUCAAAACAAAC[dT][dT] | GUUUGUUUUGAGACAGAGC[dT][dT] |
| | (SEQ ID NO: 465) | (SEQ ID NO: 151) | (SEQ ID NO: 308) |
| RAG6-90 | CTCTGTCTCAAAACAAACA | CUCUGUCUCAAAACAAACA[dT][dT] | UGUUUGUUUUGAGACAGAG[dT][dT] |
| | (SEQ ID NO: 466) | (SEQ ID NO: 152) | (SEQ ID NO: 309) |
| RAG6-45 | AACACAGTGAAATGAAAGG | AACACAGUGAAAUGAAAGG[dT][dT] | CCUUUCAUUUCACUGUGUU[dT][dT] |
| | (SEQ ID NO: 467) | (SEQ ID NO: 153) | (SEQ ID NO: 310) |
| RAG6-44 | ACACAGTGAAATGAAAGGA | ACACAGUGAAAUGAAAGGA[dT][dT] | UCCUUUCAUUUCACUGUGU[dT][dT] |
| | (SEQ ID NO: 468) | (SEQ ID NO: 154) | (SEQ ID NO: 311) |
| RAG6-41 | CAGTGAAATGAAAGGATTG | CAGUGAAAUGAAAGGAUUG[dT][dT] | CAAUCCUUUCAUUUCACUG[dT][dT] |
| | (SEQ ID NO: 469) | (SEQ ID NO: 155) | (SEQ ID NO: 312) |
| RAG6-39 | GTGAAATGAAAGGATTGAG | GUGAAAUGAAAGGAUUGAG[dT][dT] | CUCAAUCCUUUCAUUUCAC[dT][dT] |
| | (SEQ ID NO: 470) | (SEQ ID NO: 156) | (SEQ ID NO: 313) |
| RAG6-37 | GAAATGAAAGGATTGAGAG | GAAAUGAAAGGAUUGAGAG[dT][dT] | CUCUCAAUCCUUUCAUUUC[dT][dT] |
| | (SEQ ID NO: 471) | (SEQ ID NO: 157) | (SEQ ID NO: 314) |

TABLE 9
DNA sequences of saRNA hotspot regions on SMN2 promoter

| | | |
|---|---|---|
| H1 1481-1639 | agtcgcactctgtcactcaggctggagtgcagtggcgtgatcttggctcactgcaacctccg | (SEQ ID NO: 472) |
| | cctcccgagttcaagtgattctcctggctcagcct | |
| | cccaagcagctgtcattacaggcctgcaccaccacacccggctgatttttgtatttttagga | |
| H2 1008-1090 | aatactggaggcccggtgtggtggctcacacctgtaatcccagcactttgggaggccgaggc | (SEQ ID NO: 473) |
| | ggtcggattacgaggtcagg | |
| H3 180-944 | ctggccaacatggtgaaaccccatctttactaaaaatacaaaaattagccgggtgtggtggt | (SEQ ID NO: 474) |
| | gggcgcctgtaatcccagctactcggggggctga | |
| | ggcagaattgcttgaacctgggaggcagaggttgcagtgagctgagatcacgccactgcatt | |
| | ccagcctgggtgacagagcaatactctgtcgca | |
| | aaaaaaaaaaagaatactggaggctgggcgaggtggctcacacctgtaatcccagcattttg | |
| | ggatgccagaggcgggcggaatatcttgagct | |
| | caggagttcgagaccagcctacacaatatgctccaaacgccgcctctacaaaacatacagaa | |
| | actagccgggtgtggtggcgtgcccctgtggtc | |
| | ctagctacttgggaggttgaggcgggaggatcgcttgagctcgggaggtcgaggctgcaatg | |
| | agccgagatggtgccactgcactctgacgac | |
| | agagcgagactccgtctcaaaacaaacaacaaataaggttgggggatcaaatatcttctagt | |
| | gtttaaggatctgccttccttcctgcccccatgtttg | |
| | tctttccttgtttgtctttatatagatcaagcaggttttaaattcctagtaggagcttacat | |
| | ttacttttccaagggggagggggaataaatatctacacaca | |
| | cacacacacacacacacacacacacacactggagttcgagacgaggcctaagcaacatgccg | |
| | aaaccccgtctctactaaatacaaaaaatagc | |
| | tgagcttggtggcgcacgcctatagtcctagctactggggaggctg | |
| H4 37-144 | ctgcagtgagccgagatcgcgccgctgcactccagcctgagcgacagggcgaggctctgtct | (SEQ ID NO: 475) |
| | caaaacaaacaaacaaaaaaaaaaggaaaggaaatataacacagtg | |

### 8. EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it is understood by persons skilled in the relevant art that various changes in form and details can be made therein without departing from the spirit and scope of the invention.

1. A pharmaceutical composition comprising a combination of:
(a) one or more agents that increase the expression of SMN2 gene or protein, and
(b) one or more agents that increase the production of functional SMN protein by modulating SMN2 mRNA splicing or stability (SMN2 mRNA modulators).

2. The composition of claim 1, wherein at least one of the one or more agents that increase expression of SMN2 gene or protein is selected from an saRNA, a recombinant vector encoding the saRNA, and a small molecule compound.

3. The composition of claim 2, wherein the at least one of the one or more agents that increase the expression of SMN2 gene or protein is an saRNA, and wherein the saRNA comprises a sense strand and an antisense strand, or comprises a single strand, or mixtures thereof.

4. The composition of claim 1, wherein the one or more SMN2 mRNA modulators is an antisense oligonucleotide (ASO) or a small molecule compound.

5. The composition of claim 4, wherein at least one of the one or more SMN2 mRNA modulators is selected from Nusinersen, Risdiplam and Branaplam.

6. The composition of claim 2, wherein at least one saRNA comprises a first strand that is at least 90% identical in sequence to a fragment in:
(a) the region of the SMN2 gene promoter from −1639 to −1481 (SEQ ID No: 472),
(b) the region of the SMN2 gene promoter from −1090 to −1008 (SEQ ID No: 473),
(c) the region of the SMN2 gene promoter from −994 to −180 regions (SEQ ID NO: 474), or
(d) the region of the SMN2 gene promoter from −144 to −37 (SEQ ID No: 475).

7. The composition of claim 6, wherein the first strand of the saRNA has at least 75% sequence homology or complementarity with a fragment of the promoter region of the SMN2 gene that is of 16-35 nucleotides in length.

8. The composition of claim 6, wherein the first strand is 16-35 nucleotides in length, and when optimally aligned, is at least 75% identical in sequence to one of (a), (b), (c), or (d).

9. The composition of claim 6, wherein the first strand of the saRNA has at least 75% sequence homology or complementarity to a nucleotide sequence selected from the group consisting of SEQ ID NOs: 315-471.

10. The composition of claim 6, wherein the sense strand of the saRNA has at least 75% sequence homology to any of the nucleotide sequences selected from the group consisting of SEQ ID NOs: 1-157, and the antisense strand of the saRNA has at least 75% sequence homology to any of the nucleotide sequences selected from the group consisting of SEQ ID NOs: 158-314.

11. The composition of claim 10, wherein the sense strand of the saRNA comprises a nucleotide sequence selected from any one of SEQ ID NOs: 1-157, and wherein the antisense strand of the saRNA comprises a nucleotide sequence selected from any one of SEQ ID NOs: 158-314.

12. The composition of claim 6, wherein at least one nucleotide of the saRNA is a chemically modified nucleotide.

13. The composition of claim 1, wherein the composition comprises at least one pharmaceutically acceptable carrier.

14. The composition of claim 13, wherein the at least one pharmaceutically acceptable carrier is select from an aqueous carrier, liposome, polymeric polymer, and polypeptide.

15. The composition of claim 1, wherein the composition comprises a combination of:
a) 1-150 nM of the saRNA, and
b) 1-50 nM of the SMN2 mRNA modulator.

16. A method for treating or delaying the onset or progression of SMN-deficiency-related conditions in an individual, the method comprising administering to the individual an effective amount of the pharmaceutical composition of claim 1.

17. The method of claim 16, wherein the pharmaceutical composition comprises:
(a) one or more agents that increase the expression of the SMN2 gene or protein, and
(b) one or more agents that increase the production of functional SMN protein by modulating SMN2 mRNA splicing or stability (SMN2 mRNA modulators);
wherein at least one of the one or more agents that increase the expression of the SMN2 gene or protein is an saRNA, and wherein at least one of the one or more SMN2 mRNA modulators is an antisense oligonucleotide (ASO).

18. The method of claim 17, wherein at least one of the one or more SMN2 mRNA modulators is selected from Nusinersen, Risdiplam and Branaplam.

19. The method of claim 16, wherein the individual has the condition of SMA.

20. The method of claim 19, wherein the individual with SMA has decreased or abnormal SMN full length protein expression.

21. The method of claim 17, wherein at least one saRNA comprises a first strand that is at least 90% identical in sequence to a fragment in:
(a) the region of the SMN2 gene promoter from −1639 to −1481 (SEQ ID No: 472),
(b) the region of the SMN2 gene promoter from −1090 to −1008 (SEQ ID No: 473),
(c) the region of the SMN2 gene promoter from −994 to −180 regions (SEQ ID No: 474), or
(d) the region of the SMN2 gene promoter from −144 to −37 (SEQ ID No: 475).

22. The method of claim 21, wherein the first strand of the saRNA has at least 75% sequence homology or complementarity with any nucleotide sequence selected from the group consisting of SEQ ID NOs: 315-471.

23. The method of claim 21, wherein the sense strand of saRNA has at least 75% homology to any of the nucleotide sequences selected from the group consisting of SEQ ID NOs: 1-157, and the antisense strand of the saRNA has at least 75% homology to any of the nucleotide sequences selected from the group consisting of SEQ ID NOs: 158-314.

24. The method of claim 23, wherein the sense strand of the saRNA comprises a nucleotide sequence selected from any one of SEQ ID Nos: 1-157, and wherein the antisense strand of the saRNA comprises a nucleotide sequence selected from any one of SEQ ID Nos: 158-314.

25. The method of claim 21, wherein at least one nucleotide of the saRNA is a chemically modified nucleotide.


SEQUENCE LISTING (498 sequences)

SEQ ID NO: 1 - DNA, 21 bp, Artificial Sequence
  Synthetic oligonucleotide
aucugugagauguaccuuutt

SEQ ID NO: 2 - DNA, 21 bp, Artificial Sequence
  Synthetic oligonucleotide
cacucugucacucaggcugtt

SEQ ID NO: 3 - DNA, 21 bp, Artificial Sequence
  Synthetic oligonucleotide
acucugucacucaggcuggtt

SEQ ID NO: 4 - DNA, 21 bp, Artificial Sequence
  Synthetic oligonucleotide
ucugucacucaggcuggagtt

SEQ ID NO: 5 - DNA, 21 bp, Artificial Sequence
  Synthetic oligonucleotide
ucaggcuggagugcaguggtt

SEQ ID NO: 6 - DNA, 21 bp, Artificial Sequence
  Synthetic oligonucleotide
gagugcaguggcgugaucutt

SEQ ID NO: 7 - DNA, 21 bp, Artificial Sequence
  Synthetic oligonucleotide
ugcaguggcgugaucuuggtt

SEQ ID NO: 8 - DNA, 21 bp, Artificial Sequence
  Synthetic oligonucleotide
gcaguggcgugaucuuggctt

SEQ ID NO: 9 - DNA, 21 bp, Artificial Sequence
  Synthetic oligonucleotide
ggcgugaucuuggcucacutt

SEQ ID NO: 10 - DNA, 21 bp, Artificial Sequence
  Synthetic oligonucleotide
gugaucuuggcucacugcatt

SEQ ID NO: 11 - DNA, 21 bp, Artificial Sequence
  Synthetic oligonucleotide
ugaucuuggcucacugcaatt

SEQ ID NO: 12 - DNA, 21 bp, Artificial Sequence
  Synthetic oligonucleotide
aucuuggcucacugcaacctt

SEQ ID NO: 13 - DNA, 21 bp, Artificial Sequence
  Synthetic oligonucleotide
cuuggcucacugcaaccuctt

SEQ ID NO: 14 - DNA, 21 bp, Artificial Sequence
  Synthetic oligonucleotide
uuggcucacugcaaccucctt

SEQ ID NO: 15 - DNA, 21 bp, Artificial Sequence
  Synthetic oligonucleotide
gccucccgaguucaagugatt

SEQ ID NO: 16 - DNA, 21 bp, Artificial Sequence
  Synthetic oligonucleotide
ccucccgaguucaagugautt

SEQ ID NO: 17 - DNA, 21 bp, Artificial Sequence
  Synthetic oligonucleotide
cucccgaguucaagugauutt

SEQ ID NO: 18 - DNA, 21 bp, Artificial Sequence
  Synthetic oligonucleotide
ucccgaguucaagugauuctt

SEQ ID NO: 19 - DNA, 21 bp, Artificial Sequence
  Synthetic oligonucleotide
ucaagugauucuccuggcutt

SEQ ID NO: 20 - DNA, 21 bp, Artificial Sequence
  Synthetic oligonucleotide
aagugauucuccuggcucatt

SEQ ID NO: 21 - DNA, 21 bp, Artificial Sequence
  Synthetic oligonucleotide
agugauucuccuggcucagtt

SEQ ID NO: 22 - DNA, 21 bp, Artificial Sequence
  Synthetic oligonucleotide
gugauucuccuggcucagctt

SEQ ID NO: 23 - DNA, 21 bp, Artificial Sequence
  Synthetic oligonucleotide
cagccucccaagcagcugutt

SEQ ID NO: 24 - DNA, 21 bp, Artificial Sequence
  Synthetic oligonucleotide
ccucccaagcagcugucautt

SEQ ID NO: 25 - DNA, 21 bp, Artificial Sequence
  Synthetic oligonucleotide
ucccaagcagcugucauuatt

SEQ ID NO: 26 - DNA, 21 bp, Artificial Sequence
  Synthetic oligonucleotide
agcugucauuacaggccugtt

SEQ ID NO: 27 - DNA, 21 bp, Artificial Sequence
  Synthetic oligonucleotide
gcugucauuacaggccugctt

SEQ ID NO: 28 - DNA, 21 bp, Artificial Sequence
  Synthetic oligonucleotide
cugucauuacaggccugcatt

SEQ ID NO: 29 - DNA, 21 bp, Artificial Sequence
  Synthetic oligonucleotide
ggagaaacaggguuucacctt

SEQ ID NO: 30 - DNA, 21 bp, Artificial Sequence
  Synthetic oligonucleotide
agaaacaggguuucaccautt

SEQ ID NO: 31 - DNA, 21 bp, Artificial Sequence
  Synthetic oligonucleotide
aagugcugggauuauaggctt

SEQ ID NO: 32 - DNA, 21 bp, Artificial Sequence
  Synthetic oligonucleotide
uuauaggcaugagccaccgtt

SEQ ID NO: 33 - DNA, 21 bp, Artificial Sequence
  Synthetic oligonucleotide
auucuccccuuccuccacatt

SEQ ID NO: 34 - DNA, 21 bp, Artificial Sequence
  Synthetic oligonucleotide
ucuccccuuccuccacagatt

SEQ ID NO: 35 - DNA, 21 bp, Artificial Sequence
  Synthetic oligonucleotide
cauuuagcaacccuagaugtt

SEQ ID NO: 36 - DNA, 21 bp, Artificial Sequence
  Synthetic oligonucleotide
auuuagcaacccuagaugctt

SEQ ID NO: 37 - DNA, 21 bp, Artificial Sequence
  Synthetic oligonucleotide
uuuagcaacccuagaugcutt

SEQ ID NO: 38 - DNA, 21 bp, Artificial Sequence
  Synthetic oligonucleotide
uuagcaacccuagaugcuutt

SEQ ID NO: 39 - DNA, 21 bp, Artificial Sequence
  Synthetic oligonucleotide
uagcaacccuagaugcuuatt

SEQ ID NO: 40 - DNA, 21 bp, Artificial Sequence
  Synthetic oligonucleotide
auacuggaggcccggugugtt

SEQ ID NO: 41 - DNA, 21 bp, Artificial Sequence
  Synthetic oligonucleotide
gugugguggcucacaccugtt

SEQ ID NO: 42 - DNA, 21 bp, Artificial Sequence
  Synthetic oligonucleotide
ugguggcucacaccuguaatt

SEQ ID NO: 43 - DNA, 21 bp, Artificial Sequence
  Synthetic oligonucleotide
gguggcucacaccuguaautt

SEQ ID NO: 44 - DNA, 21 bp, Artificial Sequence
  Synthetic oligonucleotide
guggcucacaccuguaauctt

SEQ ID NO: 45 - DNA, 21 bp, Artificial Sequence
  Synthetic oligonucleotide
ggcucacaccuguaauccctt

SEQ ID NO: 46 - DNA, 21 bp, Artificial Sequence
  Synthetic oligonucleotide
cacaccuguaaucccagcatt

SEQ ID NO: 47 - DNA, 21 bp, Artificial Sequence
  Synthetic oligonucleotide
acaccuguaaucccagcactt

SEQ ID NO: 48 - DNA, 21 bp, Artificial Sequence
  Synthetic oligonucleotide
accuguaaucccagcacuutt

SEQ ID NO: 49 - DNA, 21 bp, Artificial Sequence
  Synthetic oligonucleotide
guaaucccagcacuuugggtt

SEQ ID NO: 50 - DNA, 21 bp, Artificial Sequence
  Synthetic oligonucleotide
uaaucccagcacuuugggatt

SEQ ID NO: 51 - DNA, 21 bp, Artificial Sequence
  Synthetic oligonucleotide
aaucccagcacuuugggagtt

SEQ ID NO: 52 - DNA, 21 bp, Artificial Sequence
  Synthetic oligonucleotide
cagcacuuugggaggccgatt

SEQ ID NO: 53 - DNA, 21 bp, Artificial Sequence
  Synthetic oligonucleotide
gaggcggucggauuacgagtt

SEQ ID NO: 54 - DNA, 21 bp, Artificial Sequence
  Synthetic oligonucleotide
ggcggucggauuacgaggutt

SEQ ID NO: 55 - DNA, 21 bp, Artificial Sequence
  Synthetic oligonucleotide
gcggucggauuacgagguctt

SEQ ID NO: 56 - DNA, 21 bp, Artificial Sequence
  Synthetic oligonucleotide
cggucggauuacgaggucatt

SEQ ID NO: 57 - DNA, 21 bp, Artificial Sequence
  Synthetic oligonucleotide
ggucggauuacgaggucagtt

SEQ ID NO: 58 - DNA, 21 bp, Artificial Sequence
  Synthetic oligonucleotide
gucggauuacgaggucaggtt

SEQ ID NO: 59 - DNA, 21 bp, Artificial Sequence
  Synthetic oligonucleotide
ucggauuacgaggucaggatt

SEQ ID NO: 60 - DNA, 21 bp, Artificial Sequence
  Synthetic oligonucleotide
cggauuacgaggucaggagtt

SEQ ID NO: 61 - DNA, 21 bp, Artificial Sequence
  Synthetic oligonucleotide
auuacgaggucaggaguuctt

SEQ ID NO: 62 - DNA, 21 bp, Artificial Sequence
  Synthetic oligonucleotide
uuacgaggucaggaguucatt

SEQ ID NO: 63 - DNA, 21 bp, Artificial Sequence
  Synthetic oligonucleotide
uacgaggucaggaguucaatt

SEQ ID NO: 64 - DNA, 21 bp, Artificial Sequence
  Synthetic oligonucleotide
acgaggucaggaguucaagtt

SEQ ID NO: 65 - DNA, 21 bp, Artificial Sequence
  Synthetic oligonucleotide
aggucaggaguucaagacctt

SEQ ID NO: 66 - DNA, 21 bp, Artificial Sequence
  Synthetic oligonucleotide
aguucaagaccagccuggctt

SEQ ID NO: 67 - DNA, 21 bp, Artificial Sequence
  Synthetic oligonucleotide
gaaaccccaucuuuacuaatt

SEQ ID NO: 68 - DNA, 21 bp, Artificial Sequence
  Synthetic oligonucleotide
auuagccgggugugguggutt

SEQ ID NO: 69 - DNA, 21 bp, Artificial Sequence
  Synthetic oligonucleotide
guggugggcgccuguaauctt

SEQ ID NO: 70 - DNA, 21 bp, Artificial Sequence
  Synthetic oligonucleotide
ggcgccuguaaucccagcutt

SEQ ID NO: 71 - DNA, 21 bp, Artificial Sequence
  Synthetic oligonucleotide
uaaucccagcuacucggggtt

SEQ ID NO: 72 - DNA, 21 bp, Artificial Sequence
  Synthetic oligonucleotide
gggcugaggcagaauugcutt

SEQ ID NO: 73 - DNA, 21 bp, Artificial Sequence
  Synthetic oligonucleotide
ggcagaauugcuugaaccutt

SEQ ID NO: 74 - DNA, 21 bp, Artificial Sequence
  Synthetic oligonucleotide
cagaauugcuugaaccuggtt

SEQ ID NO: 75 - DNA, 21 bp, Artificial Sequence
  Synthetic oligonucleotide
ugaaccugggaggcagaggtt

SEQ ID NO: 76 - DNA, 21 bp, Artificial Sequence
  Synthetic oligonucleotide
gaaccugggaggcagaggutt

SEQ ID NO: 77 - DNA, 21 bp, Artificial Sequence
  Synthetic oligonucleotide
accugggaggcagagguugtt

SEQ ID NO: 78 - DNA, 21 bp, Artificial Sequence
  Synthetic oligonucleotide
ugcagugagcugagaucactt

SEQ ID NO: 79 - DNA, 21 bp, Artificial Sequence
  Synthetic oligonucleotide
cugagaucacgccacugcatt

SEQ ID NO: 80 - DNA, 21 bp, Artificial Sequence
  Synthetic oligonucleotide
aucacgccacugcauuccatt

SEQ ID NO: 81 - DNA, 21 bp, Artificial Sequence
  Synthetic oligonucleotide
gggugacagagcaauacuctt

SEQ ID NO: 82 - DNA, 21 bp, Artificial Sequence
  Synthetic oligonucleotide
ugacagagcaauacucugutt

SEQ ID NO: 83 - DNA, 21 bp, Artificial Sequence
  Synthetic oligonucleotide
agagcaauacucugucgcatt

SEQ ID NO: 84 - DNA, 21 bp, Artificial Sequence
  Synthetic oligonucleotide
aaaagaauacuggaggcugtt

SEQ ID NO: 85 - DNA, 21 bp, Artificial Sequence
  Synthetic oligonucleotide
aaagaauacuggaggcuggtt

SEQ ID NO: 86 - DNA, 21 bp, Artificial Sequence
  Synthetic oligonucleotide
auacuggaggcugggcgagtt

SEQ ID NO: 87 - DNA, 21 bp, Artificial Sequence
  Synthetic oligonucleotide
cgagguggcucacaccugutt

SEQ ID NO: 88 - DNA, 21 bp, Artificial Sequence
  Synthetic oligonucleotide
gguggcucacaccuguaautt

SEQ ID NO: 89 - DNA, 21 bp, Artificial Sequence
  Synthetic oligonucleotide
ggcucacaccuguaauccctt

SEQ ID NO: 90 - DNA, 21 bp, Artificial Sequence
  Synthetic oligonucleotide
gcucacaccuguaaucccatt

SEQ ID NO: 91 - DNA, 21 bp, Artificial Sequence
  Synthetic oligonucleotide
cacaccuguaaucccagcatt

SEQ ID NO: 92 - DNA, 21 bp, Artificial Sequence
  Synthetic oligonucleotide
uaaucccagcauuuugggatt

SEQ ID NO: 93 - DNA, 21 bp, Artificial Sequence
  Synthetic oligonucleotide
gggcggaauaucuugagcutt

SEQ ID NO: 94 - DNA, 21 bp, Artificial Sequence
  Synthetic oligonucleotide
aauaucuugagcucaggagtt

SEQ ID NO: 95 - DNA, 21 bp, Artificial Sequence
  Synthetic oligonucleotide
uucgagaccagccuacacatt

SEQ ID NO: 96 - DNA, 21 bp, Artificial Sequence
  Synthetic oligonucleotide
ccagccuacacaauaugcutt

SEQ ID NO: 97 - DNA, 21 bp, Artificial Sequence
  Synthetic oligonucleotide
acacaauaugcuccaaacgtt

SEQ ID NO: 98 - DNA, 21 bp, Artificial Sequence
  Synthetic oligonucleotide
cacaauaugcuccaaacgctt

SEQ ID NO: 99 - DNA, 21 bp, Artificial Sequence
  Synthetic oligonucleotide
acaauaugcuccaaacgcctt

SEQ ID NO: 100 - DNA, 21 bp, Artificial Sequence
  Synthetic oligonucleotide
caaacgccgccucuacaaatt

SEQ ID NO: 101 - DNA, 21 bp, Artificial Sequence
  Synthetic oligonucleotide
cugugguccuagcuacuugtt

SEQ ID NO: 102 - DNA, 21 bp, Artificial Sequence
  Synthetic oligonucleotide
gggaggaucgcuugagcuctt

SEQ ID NO: 103 - DNA, 21 bp, Artificial Sequence
  Synthetic oligonucleotide
gaggaucgcuugagcucggtt

SEQ ID NO: 104 - DNA, 21 bp, Artificial Sequence
  Synthetic oligonucleotide
ggaggucgaggcugcaaugtt

SEQ ID NO: 105 - DNA, 21 bp, Artificial Sequence
  Synthetic oligonucleotide
ggucgaggcugcaaugagctt

SEQ ID NO: 106 - DNA, 21 bp, Artificial Sequence
  Synthetic oligonucleotide
caaugagccgagauggugctt

SEQ ID NO: 107 - DNA, 21 bp, Artificial Sequence
  Synthetic oligonucleotide
aaugagccgagauggugcctt

SEQ ID NO: 108 - DNA, 21 bp, Artificial Sequence
  Synthetic oligonucleotide
ccgagauggugccacugcatt

SEQ ID NO: 109 - DNA, 21 bp, Artificial Sequence
  Synthetic oligonucleotide
gagauggugccacugcacutt

SEQ ID NO: 110 - DNA, 21 bp, Artificial Sequence
  Synthetic oligonucleotide
agauggugccacugcacuctt

SEQ ID NO: 111 - DNA, 21 bp, Artificial Sequence
  Synthetic oligonucleotide
auggugccacugcacucugtt

SEQ ID NO: 112 - DNA, 21 bp, Artificial Sequence
  Synthetic oligonucleotide
ccacugcacucugacgacatt

SEQ ID NO: 113 - DNA, 21 bp, Artificial Sequence
  Synthetic oligonucleotide
cacugcacucugacgacagtt

SEQ ID NO: 114 - DNA, 21 bp, Artificial Sequence
  Synthetic oligonucleotide
ucugacgacagagcgagactt

SEQ ID NO: 115 - DNA, 21 bp, Artificial Sequence
  Synthetic oligonucleotide
cugacgacagagcgagacutt

SEQ ID NO: 116 - DNA, 21 bp, Artificial Sequence
  Synthetic oligonucleotide
gagacuccgucucaaaacatt

SEQ ID NO: 117 - DNA, 21 bp, Artificial Sequence
  Synthetic oligonucleotide
agacuccgucucaaaacaatt

SEQ ID NO: 118 - DNA, 21 bp, Artificial Sequence
  Synthetic oligonucleotide
ucuaguguuuaaggaucugtt

SEQ ID NO: 119 - DNA, 21 bp, Artificial Sequence
  Synthetic oligonucleotide
uaguguuuaaggaucugcctt

SEQ ID NO: 120 - DNA, 21 bp, Artificial Sequence
  Synthetic oligonucleotide
uguuuaaggaucugccuuctt

SEQ ID NO: 121 - DNA, 21 bp, Artificial Sequence
  Synthetic oligonucleotide
ggaucugccuuccuuccugtt

SEQ ID NO: 122 - DNA, 21 bp, Artificial Sequence
  Synthetic oligonucleotide
uugucuuuccuuguuuguctt

SEQ ID NO: 123 - DNA, 21 bp, Artificial Sequence
  Synthetic oligonucleotide
gucuuuccuuguuugucuutt

SEQ ID NO: 124 - DNA, 21 bp, Artificial Sequence
  Synthetic oligonucleotide
caagcagguuuuaaauucctt

SEQ ID NO: 125 - DNA, 21 bp, Artificial Sequence
  Synthetic oligonucleotide
gcagguuuuaaauuccuagtt

SEQ ID NO: 126 - DNA, 21 bp, Artificial Sequence
  Synthetic oligonucleotide
acauuuacuuuuccaagggtt

SEQ ID NO: 127 - DNA, 21 bp, Artificial Sequence
  Synthetic oligonucleotide
acacuggaguucgagacgatt

SEQ ID NO: 128 - DNA, 21 bp, Artificial Sequence
  Synthetic oligonucleotide
cuggaguucgagacgaggctt

SEQ ID NO: 129 - DNA, 21 bp, Artificial Sequence
  Synthetic oligonucleotide
uucgagacgaggccuaagctt

SEQ ID NO: 130 - DNA, 21 bp, Artificial Sequence
  Synthetic oligonucleotide
gagacgaggccuaagcaactt

SEQ ID NO: 131 - DNA, 21 bp, Artificial Sequence
  Synthetic oligonucleotide
agacgaggccuaagcaacatt

SEQ ID NO: 132 - DNA, 21 bp, Artificial Sequence
  Synthetic oligonucleotide
gacgaggccuaagcaacautt

SEQ ID NO: 133 - DNA, 21 bp, Artificial Sequence
  Synthetic oligonucleotide
ccuaagcaacaugccgaaatt

SEQ ID NO: 134 - DNA, 21 bp, Artificial Sequence
  Synthetic oligonucleotide
cuaagcaacaugccgaaactt

SEQ ID NO: 135 - DNA, 21 bp, Artificial Sequence
  Synthetic oligonucleotide
uaagcaacaugccgaaacctt

SEQ ID NO: 136 - DNA, 21 bp, Artificial Sequence
  Synthetic oligonucleotide
ugguggcgcacgccuauagtt

SEQ ID NO: 137 - DNA, 21 bp, Artificial Sequence
  Synthetic oligonucleotide
gguggcgcacgccuauagutt

SEQ ID NO: 138 - DNA, 21 bp, Artificial Sequence
  Synthetic oligonucleotide
cuauaguccuagcuacuggtt

SEQ ID NO: 139 - DNA, 21 bp, Artificial Sequence
  Synthetic oligonucleotide
uauaguccuagcuacugggtt

SEQ ID NO: 140 - DNA, 21 bp, Artificial Sequence
  Synthetic oligonucleotide
ugaggugggaggaucgcuutt

SEQ ID NO: 141 - DNA, 21 bp, Artificial Sequence
  Synthetic oligonucleotide
cugcagugagccgagaucgtt

SEQ ID NO: 142 - DNA, 21 bp, Artificial Sequence
  Synthetic oligonucleotide
ugcagugagccgagaucgctt

SEQ ID NO: 143 - DNA, 21 bp, Artificial Sequence
  Synthetic oligonucleotide
ugcacuccagccugagcgatt

SEQ ID NO: 144 - DNA, 21 bp, Artificial Sequence
  Synthetic oligonucleotide
cacuccagccugagcgacatt

SEQ ID NO: 145 - DNA, 21 bp, Artificial Sequence
  Synthetic oligonucleotide
acagggcgaggcucugucutt

SEQ ID NO: 146 - DNA, 21 bp, Artificial Sequence
  Synthetic oligonucleotide
gggcgaggcucugucucaatt

SEQ ID NO: 147 - DNA, 21 bp, Artificial Sequence
  Synthetic oligonucleotide
ggcgaggcucugucucaaatt

SEQ ID NO: 148 - DNA, 21 bp, Artificial Sequence
  Synthetic oligonucleotide
gcgaggcucugucucaaaatt

SEQ ID NO: 149 - DNA, 21 bp, Artificial Sequence
  Synthetic oligonucleotide
aggcucugucucaaaacaatt

SEQ ID NO: 150 - DNA, 21 bp, Artificial Sequence
  Synthetic oligonucleotide
ggcucugucucaaaacaaatt

SEQ ID NO: 151 - DNA, 21 bp, Artificial Sequence
  Synthetic oligonucleotide
gcucugucucaaaacaaactt

SEQ ID NO: 152 - DNA, 21 bp, Artificial Sequence
  Synthetic oligonucleotide
cucugucucaaaacaaacatt

SEQ ID NO: 153 - DNA, 21 bp, Artificial Sequence
  Synthetic oligonucleotide
aacacagugaaaugaaaggtt

SEQ ID NO: 154 - DNA, 21 bp, Artificial Sequence
  Synthetic oligonucleotide
acacagugaaaugaaaggatt

SEQ ID NO: 155 - DNA, 21 bp, Artificial Sequence
  Synthetic oligonucleotide
cagugaaaugaaaggauugtt

SEQ ID NO: 156 - DNA, 21 bp, Artificial Sequence
  Synthetic oligonucleotide
gugaaaugaaaggauugagtt

SEQ ID NO: 157 - DNA, 21 bp, Artificial Sequence
  Synthetic oligonucleotide
gaaaugaaaggauugagagtt

SEQ ID NO: 158 - DNA, 21 bp, Artificial Sequence
  Synthetic oligonucleotide
aaagguacaucucacagautt

SEQ ID NO: 159 - DNA, 21 bp, Artificial Sequence
  Synthetic oligonucleotide
cagccugagugacagagugtt

SEQ ID NO: 160 - DNA, 21 bp, Artificial Sequence
  Synthetic oligonucleotide
ccagccugagugacagagutt

SEQ ID NO: 161 - DNA, 21 bp, Artificial Sequence
  Synthetic oligonucleotide
cuccagccugagugacagatt

SEQ ID NO: 162 - DNA, 21 bp, Artificial Sequence
  Synthetic oligonucleotide
ccacugcacuccagccugatt

SEQ ID NO: 163 - DNA, 21 bp, Artificial Sequence
  Synthetic oligonucleotide
agaucacgccacugcacuctt

SEQ ID NO: 164 - DNA, 21 bp, Artificial Sequence
  Synthetic oligonucleotide
ccaagaucacgccacugcatt

SEQ ID NO: 165 - DNA, 21 bp, Artificial Sequence
  Synthetic oligonucleotide
gccaagaucacgccacugctt

SEQ ID NO: 166 - DNA, 21 bp, Artificial Sequence
  Synthetic oligonucleotide
agugagccaagaucacgcctt

SEQ ID NO: 167 - DNA, 21 bp, Artificial Sequence
  Synthetic oligonucleotide
ugcagugagccaagaucactt

SEQ ID NO: 168 - DNA, 21 bp, Artificial Sequence
  Synthetic oligonucleotide
uugcagugagccaagaucatt

SEQ ID NO: 169 - DNA, 21 bp, Artificial Sequence
  Synthetic oligonucleotide
gguugcagugagccaagautt

SEQ ID NO: 170 - DNA, 21 bp, Artificial Sequence
  Synthetic oligonucleotide
gagguugcagugagccaagtt

SEQ ID NO: 171 - DNA, 21 bp, Artificial Sequence
  Synthetic oligonucleotide
ggagguugcagugagccaatt

SEQ ID NO: 172 - DNA, 21 bp, Artificial Sequence
  Synthetic oligonucleotide
ucacuugaacucgggaggctt

SEQ ID NO: 173 - DNA, 21 bp, Artificial Sequence
  Synthetic oligonucleotide
aucacuugaacucgggaggtt

SEQ ID NO: 174 - DNA, 21 bp, Artificial Sequence
  Synthetic oligonucleotide
aaucacuugaacucgggagtt

SEQ ID NO: 175 - DNA, 21 bp, Artificial Sequence
  Synthetic oligonucleotide
gaaucacuugaacucgggatt

SEQ ID NO: 176 - DNA, 21 bp, Artificial Sequence
  Synthetic oligonucleotide
agccaggagaaucacuugatt

SEQ ID NO: 177 - DNA, 21 bp, Artificial Sequence
  Synthetic oligonucleotide
ugagccaggagaaucacuutt

SEQ ID NO: 178 - DNA, 21 bp, Artificial Sequence
  Synthetic oligonucleotide
cugagccaggagaaucacutt

SEQ ID NO: 179 - DNA, 21 bp, Artificial Sequence
  Synthetic oligonucleotide
gcugagccaggagaaucactt

SEQ ID NO: 180 - DNA, 21 bp, Artificial Sequence
  Synthetic oligonucleotide
acagcugcuugggaggcugtt

SEQ ID NO: 181 - DNA, 21 bp, Artificial Sequence
  Synthetic oligonucleotide
augacagcugcuugggaggtt

SEQ ID NO: 182 - DNA, 21 bp, Artificial Sequence
  Synthetic oligonucleotide
uaaugacagcugcuugggatt

SEQ ID NO: 183 - DNA, 21 bp, Artificial Sequence
  Synthetic oligonucleotide
caggccuguaaugacagcutt

SEQ ID NO: 184 - DNA, 21 bp, Artificial Sequence
  Synthetic oligonucleotide
gcaggccuguaaugacagctt

SEQ ID NO: 185 - DNA, 21 bp, Artificial Sequence
  Synthetic oligonucleotide
ugcaggccuguaaugacagtt

SEQ ID NO: 186 - DNA, 21 bp, Artificial Sequence
  Synthetic oligonucleotide
ggugaaacccuguuucucctt

SEQ ID NO: 187 - DNA, 21 bp, Artificial Sequence
  Synthetic oligonucleotide
auggugaaacccuguuucutt

SEQ ID NO: 188 - DNA, 21 bp, Artificial Sequence
  Synthetic oligonucleotide
gccuauaaucccagcacuutt

SEQ ID NO: 189 - DNA, 21 bp, Artificial Sequence
  Synthetic oligonucleotide
cgguggcucaugccuauaatt

SEQ ID NO: 190 - DNA, 21 bp, Artificial Sequence
  Synthetic oligonucleotide
uguggaggaaggggagaautt

SEQ ID NO: 191 - DNA, 21 bp, Artificial Sequence
  Synthetic oligonucleotide
ucuguggaggaaggggagatt

SEQ ID NO: 192 - DNA, 21 bp, Artificial Sequence
  Synthetic oligonucleotide
caucuaggguugcuaaaugtt

SEQ ID NO: 193 - DNA, 21 bp, Artificial Sequence
  Synthetic oligonucleotide
gcaucuaggguugcuaaautt

SEQ ID NO: 194 - DNA, 21 bp, Artificial Sequence
  Synthetic oligonucleotide
agcaucuaggguugcuaaatt

SEQ ID NO: 195 - DNA, 21 bp, Artificial Sequence
  Synthetic oligonucleotide
aagcaucuaggguugcuaatt

SEQ ID NO: 196 - DNA, 21 bp, Artificial Sequence
  Synthetic oligonucleotide
uaagcaucuaggguugcuatt

SEQ ID NO: 197 - DNA, 21 bp, Artificial Sequence
  Synthetic oligonucleotide
cacaccgggccuccaguautt

SEQ ID NO: 198 - DNA, 21 bp, Artificial Sequence
  Synthetic oligonucleotide
caggugugagccaccacactt

SEQ ID NO: 199 - DNA, 21 bp, Artificial Sequence
  Synthetic oligonucleotide
uuacaggugugagccaccatt

SEQ ID NO: 200 - DNA, 21 bp, Artificial Sequence
  Synthetic oligonucleotide
auuacaggugugagccacctt

SEQ ID NO: 201 - DNA, 21 bp, Artificial Sequence
  Synthetic oligonucleotide
gauuacaggugugagccactt

SEQ ID NO: 202 - DNA, 21 bp, Artificial Sequence
  Synthetic oligonucleotide
gggauuacaggugugagcctt

SEQ ID NO: 203 - DNA, 21 bp, Artificial Sequence
  Synthetic oligonucleotide
ugcugggauuacaggugugtt

SEQ ID NO: 204 - DNA, 21 bp, Artificial Sequence
  Synthetic oligonucleotide
gugcugggauuacaggugutt

SEQ ID NO: 205 - DNA, 21 bp, Artificial Sequence
  Synthetic oligonucleotide
aagugcugggauuacaggutt

SEQ ID NO: 206 - DNA, 21 bp, Artificial Sequence
  Synthetic oligonucleotide
cccaaagugcugggauuactt

SEQ ID NO: 207 - DNA, 21 bp, Artificial Sequence
  Synthetic oligonucleotide
ucccaaagugcugggauuatt

SEQ ID NO: 208 - DNA, 21 bp, Artificial Sequence
  Synthetic oligonucleotide
cucccaaagugcugggauutt

SEQ ID NO: 209 - DNA, 21 bp, Artificial Sequence
  Synthetic oligonucleotide
ucggccucccaaagugcugtt

SEQ ID NO: 210 - DNA, 21 bp, Artificial Sequence
  Synthetic oligonucleotide
cucguaauccgaccgccuctt

SEQ ID NO: 211 - DNA, 21 bp, Artificial Sequence
  Synthetic oligonucleotide
accucguaauccgaccgcctt

SEQ ID NO: 212 - DNA, 21 bp, Artificial Sequence
  Synthetic oligonucleotide
gaccucguaauccgaccgctt

SEQ ID NO: 213 - DNA, 21 bp, Artificial Sequence
  Synthetic oligonucleotide
ugaccucguaauccgaccgtt

SEQ ID NO: 214 - DNA, 21 bp, Artificial Sequence
  Synthetic oligonucleotide
cugaccucguaauccgacctt

SEQ ID NO: 215 - DNA, 21 bp, Artificial Sequence
  Synthetic oligonucleotide
ccugaccucguaauccgactt

SEQ ID NO: 216 - DNA, 21 bp, Artificial Sequence
  Synthetic oligonucleotide
uccugaccucguaauccgatt

SEQ ID NO: 217 - DNA, 21 bp, Artificial Sequence
  Synthetic oligonucleotide
cuccugaccucguaauccgtt

SEQ ID NO: 218 - DNA, 21 bp, Artificial Sequence
  Synthetic oligonucleotide
gaacuccugaccucguaautt

SEQ ID NO: 219 - DNA, 21 bp, Artificial Sequence
  Synthetic oligonucleotide
ugaacuccugaccucguaatt

SEQ ID NO: 220 - DNA, 21 bp, Artificial Sequence
  Synthetic oligonucleotide
uugaacuccugaccucguatt

SEQ ID NO: 221 - DNA, 21 bp, Artificial Sequence
  Synthetic oligonucleotide
cuugaacuccugaccucgutt

SEQ ID NO: 222 - DNA, 21 bp, Artificial Sequence
  Synthetic oligonucleotide
ggucuugaacuccugaccutt

SEQ ID NO: 223 - DNA, 21 bp, Artificial Sequence
  Synthetic oligonucleotide
gccaggcuggucuugaacutt

SEQ ID NO: 224 - DNA, 21 bp, Artificial Sequence
  Synthetic oligonucleotide
uuaguaaagaugggguuuctt

SEQ ID NO: 225 - DNA, 21 bp, Artificial Sequence
  Synthetic oligonucleotide
accaccacacccggcuaautt

SEQ ID NO: 226 - DNA, 21 bp, Artificial Sequence
  Synthetic oligonucleotide
gauuacaggcgcccaccactt

SEQ ID NO: 227 - DNA, 21 bp, Artificial Sequence
  Synthetic oligonucleotide
agcugggauuacaggcgcctt

SEQ ID NO: 228 - DNA, 21 bp, Artificial Sequence
  Synthetic oligonucleotide
ccccgaguagcugggauuatt

SEQ ID NO: 229 - DNA, 21 bp, Artificial Sequence
  Synthetic oligonucleotide
agcaauucugccucagccctt

SEQ ID NO: 230 - DNA, 21 bp, Artificial Sequence
  Synthetic oligonucleotide
agguucaagcaauucugcctt

SEQ ID NO: 231 - DNA, 21 bp, Artificial Sequence
  Synthetic oligonucleotide
ccagguucaagcaauucugtt

SEQ ID NO: 232 - DNA, 21 bp, Artificial Sequence
  Synthetic oligonucleotide
ccucugccucccagguucatt

SEQ ID NO: 233 - DNA, 21 bp, Artificial Sequence
  Synthetic oligonucleotide
accucugccucccagguuctt

SEQ ID NO: 234 - DNA, 21 bp, Artificial Sequence
  Synthetic oligonucleotide
caaccucugccucccaggutt

SEQ ID NO: 235 - DNA, 21 bp, Artificial Sequence
  Synthetic oligonucleotide
gugaucucagcucacugcatt

SEQ ID NO: 236 - DNA, 21 bp, Artificial Sequence
  Synthetic oligonucleotide
ugcaguggcgugaucucagtt

SEQ ID NO: 237 - DNA, 21 bp, Artificial Sequence
  Synthetic oligonucleotide
uggaaugcaguggcgugautt

SEQ ID NO: 238 - DNA, 21 bp, Artificial Sequence
  Synthetic oligonucleotide
gaguauugcucugucaccctt

SEQ ID NO: 239 - DNA, 21 bp, Artificial Sequence
  Synthetic oligonucleotide
acagaguauugcucugucatt

SEQ ID NO: 240 - DNA, 21 bp, Artificial Sequence
  Synthetic oligonucleotide
ugcgacagaguauugcucutt

SEQ ID NO: 241 - DNA, 21 bp, Artificial Sequence
  Synthetic oligonucleotide
cagccuccaguauucuuuutt

SEQ ID NO: 242 - DNA, 21 bp, Artificial Sequence
  Synthetic oligonucleotide
ccagccuccaguauucuuutt

SEQ ID NO: 243 - DNA, 21 bp, Artificial Sequence
  Synthetic oligonucleotide
cucgcccagccuccaguautt

SEQ ID NO: 244 - DNA, 21 bp, Artificial Sequence
  Synthetic oligonucleotide
acaggugugagccaccucgtt

SEQ ID NO: 245 - DNA, 21 bp, Artificial Sequence
  Synthetic oligonucleotide
auuacaggugugagccacctt

SEQ ID NO: 246 - DNA, 21 bp, Artificial Sequence
  Synthetic oligonucleotide
gggauuacaggugugagcctt

SEQ ID NO: 247 - DNA, 21 bp, Artificial Sequence
  Synthetic oligonucleotide
ugggauuacaggugugagctt

SEQ ID NO: 248 - DNA, 21 bp, Artificial Sequence
  Synthetic oligonucleotide
ugcugggauuacaggugugtt

SEQ ID NO: 249 - DNA, 21 bp, Artificial Sequence
  Synthetic oligonucleotide
ucccaaaaugcugggauuatt

SEQ ID NO: 250 - DNA, 21 bp, Artificial Sequence
  Synthetic oligonucleotide
agcucaagauauuccgccctt

SEQ ID NO: 251 - DNA, 21 bp, Artificial Sequence
  Synthetic oligonucleotide
cuccugagcucaagauauutt

SEQ ID NO: 252 - DNA, 21 bp, Artificial Sequence
  Synthetic oligonucleotide
uguguaggcuggucucgaatt

SEQ ID NO: 253 - DNA, 21 bp, Artificial Sequence
  Synthetic oligonucleotide
agcauauuguguaggcuggtt

SEQ ID NO: 254 - DNA, 21 bp, Artificial Sequence
  Synthetic oligonucleotide
cguuuggagcauauugugutt

SEQ ID NO: 255 - DNA, 21 bp, Artificial Sequence
  Synthetic oligonucleotide
gcguuuggagcauauugugtt

SEQ ID NO: 256 - DNA, 21 bp, Artificial Sequence
  Synthetic oligonucleotide
ggcguuuggagcauauugutt

SEQ ID NO: 257 - DNA, 21 bp, Artificial Sequence
  Synthetic oligonucleotide
uuuguagaggcggcguuugtt

SEQ ID NO: 258 - DNA, 21 bp, Artificial Sequence
  Synthetic oligonucleotide
caaguagcuaggaccacagtt

SEQ ID NO: 259 - DNA, 21 bp, Artificial Sequence
  Synthetic oligonucleotide
gagcucaagcgauccuccctt

SEQ ID NO: 260 - DNA, 21 bp, Artificial Sequence
  Synthetic oligonucleotide
ccgagcucaagcgauccuctt

SEQ ID NO: 261 - DNA, 21 bp, Artificial Sequence
  Synthetic oligonucleotide
cauugcagccucgaccucctt

SEQ ID NO: 262 - DNA, 21 bp, Artificial Sequence
  Synthetic oligonucleotide
gcucauugcagccucgacctt

SEQ ID NO: 263 - DNA, 21 bp, Artificial Sequence
  Synthetic oligonucleotide
gcaccaucucggcucauugtt

SEQ ID NO: 264 - DNA, 21 bp, Artificial Sequence
  Synthetic oligonucleotide
ggcaccaucucggcucauutt

SEQ ID NO: 265 - DNA, 21 bp, Artificial Sequence
  Synthetic oligonucleotide
ugcaguggcaccaucucggtt

SEQ ID NO: 266 - DNA, 21 bp, Artificial Sequence
  Synthetic oligonucleotide
agugcaguggcaccaucuctt

SEQ ID NO: 267 - DNA, 21 bp, Artificial Sequence
  Synthetic oligonucleotide
gagugcaguggcaccaucutt

SEQ ID NO: 268 - DNA, 21 bp, Artificial Sequence
  Synthetic oligonucleotide
cagagugcaguggcaccautt

SEQ ID NO: 269 - DNA, 21 bp, Artificial Sequence
  Synthetic oligonucleotide
ugucgucagagugcaguggtt

SEQ ID NO: 270 - DNA, 21 bp, Artificial Sequence
  Synthetic oligonucleotide
cugucgucagagugcagugtt

SEQ ID NO: 271 - DNA, 21 bp, Artificial Sequence
  Synthetic oligonucleotide
gucucgcucugucgucagatt

SEQ ID NO: 272 - DNA, 21 bp, Artificial Sequence
  Synthetic oligonucleotide
agucucgcucugucgucagtt

SEQ ID NO: 273 - DNA, 21 bp, Artificial Sequence
  Synthetic oligonucleotide
uguuuugagacggagucuctt

SEQ ID NO: 274 - DNA, 21 bp, Artificial Sequence
  Synthetic oligonucleotide
uuguuuugagacggagucutt

SEQ ID NO: 275 - DNA, 21 bp, Artificial Sequence
  Synthetic oligonucleotide
cagauccuuaaacacuagatt

SEQ ID NO: 276 - DNA, 21 bp, Artificial Sequence
  Synthetic oligonucleotide
ggcagauccuuaaacacuatt

SEQ ID NO: 277 - DNA, 21 bp, Artificial Sequence
  Synthetic oligonucleotide
gaaggcagauccuuaaacatt

SEQ ID NO: 278 - DNA, 21 bp, Artificial Sequence
  Synthetic oligonucleotide
caggaaggaaggcagaucctt

SEQ ID NO: 279 - DNA, 21 bp, Artificial Sequence
  Synthetic oligonucleotide
gacaaacaaggaaagacaatt

SEQ ID NO: 280 - DNA, 21 bp, Artificial Sequence
  Synthetic oligonucleotide
aagacaaacaaggaaagactt

SEQ ID NO: 281 - DNA, 21 bp, Artificial Sequence
  Synthetic oligonucleotide
ggaauuuaaaaccugcuugtt

SEQ ID NO: 282 - DNA, 21 bp, Artificial Sequence
  Synthetic oligonucleotide
cuaggaauuuaaaaccugctt

SEQ ID NO: 283 - DNA, 21 bp, Artificial Sequence
  Synthetic oligonucleotide
cccuuggaaaaguaaaugutt

SEQ ID NO: 284 - DNA, 21 bp, Artificial Sequence
  Synthetic oligonucleotide
ucgucucgaacuccagugutt

SEQ ID NO: 285 - DNA, 21 bp, Artificial Sequence
  Synthetic oligonucleotide
gccucgucucgaacuccagtt

SEQ ID NO: 286 - DNA, 21 bp, Artificial Sequence
  Synthetic oligonucleotide
gcuuaggccucgucucgaatt

SEQ ID NO: 287 - DNA, 21 bp, Artificial Sequence
  Synthetic oligonucleotide
guugcuuaggccucgucuctt

SEQ ID NO: 288 - DNA, 21 bp, Artificial Sequence
  Synthetic oligonucleotide
uguugcuuaggccucgucutt

SEQ ID NO: 289 - DNA, 21 bp, Artificial Sequence
  Synthetic oligonucleotide
auguugcuuaggccucguctt

SEQ ID NO: 290 - DNA, 21 bp, Artificial Sequence
  Synthetic oligonucleotide
uuucggcauguugcuuaggtt

SEQ ID NO: 291 - DNA, 21 bp, Artificial Sequence
  Synthetic oligonucleotide
guuucggcauguugcuuagtt

SEQ ID NO: 292 - DNA, 21 bp, Artificial Sequence
  Synthetic oligonucleotide
gguuucggcauguugcuuatt

SEQ ID NO: 293 - DNA, 21 bp, Artificial Sequence
  Synthetic oligonucleotide
cuauaggcgugcgccaccatt

SEQ ID NO: 294 - DNA, 21 bp, Artificial Sequence
  Synthetic oligonucleotide
acuauaggcgugcgccacctt

SEQ ID NO: 295 - DNA, 21 bp, Artificial Sequence
  Synthetic oligonucleotide
ccaguagcuaggacuauagtt

SEQ ID NO: 296 - DNA, 21 bp, Artificial Sequence
  Synthetic oligonucleotide
cccaguagcuaggacuauatt

SEQ ID NO: 297 - DNA, 21 bp, Artificial Sequence
  Synthetic oligonucleotide
aagcgauccucccaccucatt

SEQ ID NO: 298 - DNA, 21 bp, Artificial Sequence
  Synthetic oligonucleotide
cgaucucggcucacugcagtt

SEQ ID NO: 299 - DNA, 21 bp, Artificial Sequence
  Synthetic oligonucleotide
gcgaucucggcucacugcatt

SEQ ID NO: 300 - DNA, 21 bp, Artificial Sequence
  Synthetic oligonucleotide
ucgcucaggcuggagugcatt

SEQ ID NO: 301 - DNA, 21 bp, Artificial Sequence
  Synthetic oligonucleotide
ugucgcucaggcuggagugtt

SEQ ID NO: 302 - DNA, 21 bp, Artificial Sequence
  Synthetic oligonucleotide
agacagagccucgcccugutt

SEQ ID NO: 303 - DNA, 21 bp, Artificial Sequence
  Synthetic oligonucleotide
uugagacagagccucgccctt

SEQ ID NO: 304 - DNA, 21 bp, Artificial Sequence
  Synthetic oligonucleotide
uuugagacagagccucgcctt

SEQ ID NO: 305 - DNA, 21 bp, Artificial Sequence
  Synthetic oligonucleotide
uuuugagacagagccucgctt

SEQ ID NO: 306 - DNA, 21 bp, Artificial Sequence
  Synthetic oligonucleotide
uuguuuugagacagagccutt

SEQ ID NO: 307 - DNA, 21 bp, Artificial Sequence
  Synthetic oligonucleotide
uuuguuuugagacagagcctt

SEQ ID NO: 308 - DNA, 21 bp, Artificial Sequence
  Synthetic oligonucleotide
guuuguuuugagacagagctt

SEQ ID NO: 309 - DNA, 21 bp, Artificial Sequence
  Synthetic oligonucleotide
uguuuguuuugagacagagtt

SEQ ID NO: 310 - DNA, 21 bp, Artificial Sequence
  Synthetic oligonucleotide
ccuuucauuucacuguguutt

SEQ ID NO: 311 - DNA, 21 bp, Artificial Sequence
  Synthetic oligonucleotide
uccuuucauuucacugugutt

SEQ ID NO: 312 - DNA, 21 bp, Artificial Sequence
  Synthetic oligonucleotide
caauccuuucauuucacugtt

SEQ ID NO: 313 - DNA, 21 bp, Artificial Sequence
  Synthetic oligonucleotide
cucaauccuuucauuucactt

SEQ ID NO: 314 - DNA, 21 bp, Artificial Sequence
  Synthetic oligonucleotide
cucucaauccuuucauuuctt

SEQ ID NO: 315 - DNA, 19 bp, Artificial Sequence
  Synthetic oligonucleotide
atctgtgagatgtaccttt

SEQ ID NO: 316 - DNA, 19 bp, Artificial Sequence
  Synthetic oligonucleotide
cactctgtcactcaggctg

SEQ ID NO: 317 - DNA, 19 bp, Artificial Sequence
  Synthetic oligonucleotide
actctgtcactcaggctgg

SEQ ID NO: 318 - DNA, 19 bp, Artificial Sequence
  Synthetic oligonucleotide
tctgtcactcaggctggag

SEQ ID NO: 319 - DNA, 19 bp, Artificial Sequence
  Synthetic oligonucleotide
tcaggctggagtgcagtgg

SEQ ID NO: 320 - DNA, 19 bp, Artificial Sequence
  Synthetic oligonucleotide
gagtgcagtggcgtgatct

SEQ ID NO: 321 - DNA, 19 bp, Artificial Sequence
  Synthetic oligonucleotide
tgcagtggcgtgatcttgg

SEQ ID NO: 322 - DNA, 19 bp, Artificial Sequence
  Synthetic oligonucleotide
gcagtggcgtgatcttggc

SEQ ID NO: 323 - DNA, 19 bp, Artificial Sequence
  Synthetic oligonucleotide
ggcgtgatcttggctcact

SEQ ID NO: 324 - DNA, 19 bp, Artificial Sequence
  Synthetic oligonucleotide
gtgatcttggctcactgca

SEQ ID NO: 325 - DNA, 19 bp, Artificial Sequence
  Synthetic oligonucleotide
tgatcttggctcactgcaa

SEQ ID NO: 326 - DNA, 19 bp, Artificial Sequence
  Synthetic oligonucleotide
atcttggctcactgcaacc

SEQ ID NO: 327 - DNA, 19 bp, Artificial Sequence
  Synthetic oligonucleotide
cttggctcactgcaacctc

SEQ ID NO: 328 - DNA, 19 bp, Artificial Sequence
  Synthetic oligonucleotide
ttggctcactgcaacctcc

SEQ ID NO: 329 - DNA, 19 bp, Artificial Sequence
  Synthetic oligonucleotide
gcctcccgagttcaagtga

SEQ ID NO: 330 - DNA, 19 bp, Artificial Sequence
  Synthetic oligonucleotide
cctcccgagttcaagtgat

SEQ ID NO: 331 - DNA, 19 bp, Artificial Sequence
  Synthetic oligonucleotide
ctcccgagttcaagtgatt

SEQ ID NO: 332 - DNA, 19 bp, Artificial Sequence
  Synthetic oligonucleotide
tcccgagttcaagtgattc

SEQ ID NO: 333 - DNA, 19 bp, Artificial Sequence
  Synthetic oligonucleotide
tcaagtgattctcctggct

SEQ ID NO: 334 - DNA, 19 bp, Artificial Sequence
  Synthetic oligonucleotide
aagtgattctcctggctca

SEQ ID NO: 335 - DNA, 19 bp, Artificial Sequence
  Synthetic oligonucleotide
agtgattctcctggctcag

SEQ ID NO: 336 - DNA, 19 bp, Artificial Sequence
  Synthetic oligonucleotide
gtgattctcctggctcagc

SEQ ID NO: 337 - DNA, 19 bp, Artificial Sequence
  Synthetic oligonucleotide
cagcctcccaagcagctgt

SEQ ID NO: 338 - DNA, 19 bp, Artificial Sequence
  Synthetic oligonucleotide
cctcccaagcagctgtcat

SEQ ID NO: 339 - DNA, 19 bp, Artificial Sequence
  Synthetic oligonucleotide
tcccaagcagctgtcatta

SEQ ID NO: 340 - DNA, 19 bp, Artificial Sequence
  Synthetic oligonucleotide
agctgtcattacaggcctg

SEQ ID NO: 341 - DNA, 19 bp, Artificial Sequence
  Synthetic oligonucleotide
gctgtcattacaggcctgc

SEQ ID NO: 342 - DNA, 19 bp, Artificial Sequence
  Synthetic oligonucleotide
ctgtcattacaggcctgca

SEQ ID NO: 343 - DNA, 19 bp, Artificial Sequence
  Synthetic oligonucleotide
ggagaaacagggtttcacc

SEQ ID NO: 344 - DNA, 19 bp, Artificial Sequence
  Synthetic oligonucleotide
agaaacagggtttcaccat

SEQ ID NO: 345 - DNA, 19 bp, Artificial Sequence
  Synthetic oligonucleotide
aagtgctgggattataggc

SEQ ID NO: 346 - DNA, 19 bp, Artificial Sequence
  Synthetic oligonucleotide
ttataggcatgagccaccg

SEQ ID NO: 347 - DNA, 19 bp, Artificial Sequence
  Synthetic oligonucleotide
attctccccttcctccaca

SEQ ID NO: 348 - DNA, 19 bp, Artificial Sequence
  Synthetic oligonucleotide
tctccccttcctccacaga

SEQ ID NO: 349 - DNA, 19 bp, Artificial Sequence
  Synthetic oligonucleotide
catttagcaaccctagatg

SEQ ID NO: 350 - DNA, 19 bp, Artificial Sequence
  Synthetic oligonucleotide
atttagcaaccctagatgc

SEQ ID NO: 351 - DNA, 19 bp, Artificial Sequence
  Synthetic oligonucleotide
tttagcaaccctagatgct

SEQ ID NO: 352 - DNA, 19 bp, Artificial Sequence
  Synthetic oligonucleotide
ttagcaaccctagatgctt

SEQ ID NO: 353 - DNA, 19 bp, Artificial Sequence
  Synthetic oligonucleotide
tagcaaccctagatgctta

SEQ ID NO: 354 - DNA, 19 bp, Artificial Sequence
  Synthetic oligonucleotide
atactggaggcccggtgtg

SEQ ID NO: 355 - DNA, 19 bp, Artificial Sequence
  Synthetic oligonucleotide
gtgtggtggctcacacctg

SEQ ID NO: 356 - DNA, 19 bp, Artificial Sequence
  Synthetic oligonucleotide
tggtggctcacacctgtaa

SEQ ID NO: 357 - DNA, 19 bp, Artificial Sequence
  Synthetic oligonucleotide
ggtggctcacacctgtaat

SEQ ID NO: 358 - DNA, 19 bp, Artificial Sequence
  Synthetic oligonucleotide
gtggctcacacctgtaatc

SEQ ID NO: 359 - DNA, 19 bp, Artificial Sequence
  Synthetic oligonucleotide
ggctcacacctgtaatccc

SEQ ID NO: 360 - DNA, 19 bp, Artificial Sequence
  Synthetic oligonucleotide
cacacctgtaatcccagca

SEQ ID NO: 361 - DNA, 19 bp, Artificial Sequence
  Synthetic oligonucleotide
acacctgtaatcccagcac

SEQ ID NO: 362 - DNA, 19 bp, Artificial Sequence
  Synthetic oligonucleotide
acctgtaatcccagcactt

SEQ ID NO: 363 - DNA, 19 bp, Artificial Sequence
  Synthetic oligonucleotide
gtaatcccagcactttggg

SEQ ID NO: 364 - DNA, 19 bp, Artificial Sequence
  Synthetic oligonucleotide
taatcccagcactttggga

SEQ ID NO: 365 - DNA, 19 bp, Artificial Sequence
  Synthetic oligonucleotide
aatcccagcactttgggag

SEQ ID NO: 366 - DNA, 19 bp, Artificial Sequence
  Synthetic oligonucleotide
cagcactttgggaggccga

SEQ ID NO: 367 - DNA, 19 bp, Artificial Sequence
  Synthetic oligonucleotide
gaggcggtcggattacgag

SEQ ID NO: 368 - DNA, 19 bp, Artificial Sequence
  Synthetic oligonucleotide
ggcggtcggattacgaggt

SEQ ID NO: 369 - DNA, 19 bp, Artificial Sequence
  Synthetic oligonucleotide
gcggtcggattacgaggtc

SEQ ID NO: 370 - DNA, 19 bp, Artificial Sequence
  Synthetic oligonucleotide
cggtcggattacgaggtca

SEQ ID NO: 371 - DNA, 19 bp, Artificial Sequence
  Synthetic oligonucleotide
ggtcggattacgaggtcag

SEQ ID NO: 372 - DNA, 19 bp, Artificial Sequence
  Synthetic oligonucleotide
gtcggattacgaggtcagg

SEQ ID NO: 373 - DNA, 19 bp, Artificial Sequence
  Synthetic oligonucleotide
tcggattacgaggtcagga

SEQ ID NO: 374 - DNA, 19 bp, Artificial Sequence
  Synthetic oligonucleotide
cggattacgaggtcaggag

SEQ ID NO: 375 - DNA, 19 bp, Artificial Sequence
  Synthetic oligonucleotide
attacgaggtcaggagttc

SEQ ID NO: 376 - DNA, 19 bp, Artificial Sequence
  Synthetic oligonucleotide
ttacgaggtcaggagttca

SEQ ID NO: 377 - DNA, 19 bp, Artificial Sequence
  Synthetic oligonucleotide
tacgaggtcaggagttcaa

SEQ ID NO: 378 - DNA, 19 bp, Artificial Sequence
  Synthetic oligonucleotide
acgaggtcaggagttcaag

SEQ ID NO: 379 - DNA, 19 bp, Artificial Sequence
  Synthetic oligonucleotide
aggtcaggagttcaagacc

SEQ ID NO: 380 - DNA, 19 bp, Artificial Sequence
  Synthetic oligonucleotide
agttcaagaccagcctggc

SEQ ID NO: 381 - DNA, 19 bp, Artificial Sequence
  Synthetic oligonucleotide
gaaaccccatctttactaa

SEQ ID NO: 382 - DNA, 19 bp, Artificial Sequence
  Synthetic oligonucleotide
attagccgggtgtggtggt

SEQ ID NO: 383 - DNA, 19 bp, Artificial Sequence
  Synthetic oligonucleotide
gtggtgggcgcctgtaatc

SEQ ID NO: 384 - DNA, 19 bp, Artificial Sequence
  Synthetic oligonucleotide
ggcgcctgtaatcccagct

SEQ ID NO: 385 - DNA, 19 bp, Artificial Sequence
  Synthetic oligonucleotide
taatcccagctactcgggg

SEQ ID NO: 386 - DNA, 19 bp, Artificial Sequence
  Synthetic oligonucleotide
gggctgaggcagaattgct

SEQ ID NO: 387 - DNA, 19 bp, Artificial Sequence
  Synthetic oligonucleotide
ggcagaattgcttgaacct

SEQ ID NO: 388 - DNA, 19 bp, Artificial Sequence
  Synthetic oligonucleotide
cagaattgcttgaacctgg

SEQ ID NO: 389 - DNA, 19 bp, Artificial Sequence
  Synthetic oligonucleotide
tgaacctgggaggcagagg

SEQ ID NO: 390 - DNA, 19 bp, Artificial Sequence
  Synthetic oligonucleotide
gaacctgggaggcagaggt

SEQ ID NO: 391 - DNA, 19 bp, Artificial Sequence
  Synthetic oligonucleotide
acctgggaggcagaggttg

SEQ ID NO: 392 - DNA, 19 bp, Artificial Sequence
  Synthetic oligonucleotide
tgcagtgagctgagatcac

SEQ ID NO: 393 - DNA, 19 bp, Artificial Sequence
  Synthetic oligonucleotide
ctgagatcacgccactgca

SEQ ID NO: 394 - DNA, 19 bp, Artificial Sequence
  Synthetic oligonucleotide
atcacgccactgcattcca

SEQ ID NO: 395 - DNA, 19 bp, Artificial Sequence
  Synthetic oligonucleotide
gggtgacagagcaatactc

SEQ ID NO: 396 - DNA, 19 bp, Artificial Sequence
  Synthetic oligonucleotide
tgacagagcaatactctgt

SEQ ID NO: 397 - DNA, 19 bp, Artificial Sequence
  Synthetic oligonucleotide
agagcaatactctgtcgca

SEQ ID NO: 398 - DNA, 19 bp, Artificial Sequence
  Synthetic oligonucleotide
aaaagaatactggaggctg

SEQ ID NO: 399 - DNA, 19 bp, Artificial Sequence
  Synthetic oligonucleotide
aaagaatactggaggctgg

SEQ ID NO: 400 - DNA, 19 bp, Artificial Sequence
  Synthetic oligonucleotide
atactggaggctgggcgag

SEQ ID NO: 401 - DNA, 19 bp, Artificial Sequence
  Synthetic oligonucleotide
cgaggtggctcacacctgt

SEQ ID NO: 402 - DNA, 19 bp, Artificial Sequence
  Synthetic oligonucleotide
ggtggctcacacctgtaat

SEQ ID NO: 403 - DNA, 19 bp, Artificial Sequence
  Synthetic oligonucleotide
ggctcacacctgtaatccc

SEQ ID NO: 404 - DNA, 19 bp, Artificial Sequence
  Synthetic oligonucleotide
gctcacacctgtaatccca

SEQ ID NO: 405 - DNA, 19 bp, Artificial Sequence
  Synthetic oligonucleotide
cacacctgtaatcccagca

SEQ ID NO: 406 - DNA, 19 bp, Artificial Sequence
  Synthetic oligonucleotide
taatcccagcattttggga

SEQ ID NO: 407 - DNA, 19 bp, Artificial Sequence
  Synthetic oligonucleotide
gggcggaatatcttgagct

SEQ ID NO: 408 - DNA, 19 bp, Artificial Sequence
  Synthetic oligonucleotide
aatatcttgagctcaggag

SEQ ID NO: 409 - DNA, 19 bp, Artificial Sequence
  Synthetic oligonucleotide
ttcgagaccagcctacaca

SEQ ID NO: 410 - DNA, 19 bp, Artificial Sequence
  Synthetic oligonucleotide
ccagcctacacaatatgct

SEQ ID NO: 411 - DNA, 19 bp, Artificial Sequence
  Synthetic oligonucleotide
acacaatatgctccaaacg

SEQ ID NO: 412 - DNA, 19 bp, Artificial Sequence
  Synthetic oligonucleotide
cacaatatgctccaaacgc

SEQ ID NO: 413 - DNA, 19 bp, Artificial Sequence
  Synthetic oligonucleotide
acaatatgctccaaacgcc

SEQ ID NO: 414 - DNA, 19 bp, Artificial Sequence
  Synthetic oligonucleotide
caaacgccgcctctacaaa

SEQ ID NO: 415 - DNA, 19 bp, Artificial Sequence
  Synthetic oligonucleotide
ctgtggtcctagctacttg

SEQ ID NO: 416 - DNA, 19 bp, Artificial Sequence
  Synthetic oligonucleotide
gggaggatcgcttgagctc

SEQ ID NO: 417 - DNA, 19 bp, Artificial Sequence
  Synthetic oligonucleotide
gaggatcgcttgagctcgg

SEQ ID NO: 418 - DNA, 19 bp, Artificial Sequence
  Synthetic oligonucleotide
ggaggtcgaggctgcaatg

SEQ ID NO: 419 - DNA, 19 bp, Artificial Sequence
  Synthetic oligonucleotide
ggtcgaggctgcaatgagc

SEQ ID NO: 420 - DNA, 19 bp, Artificial Sequence
  Synthetic oligonucleotide
caatgagccgagatggtgc

SEQ ID NO: 421 - DNA, 19 bp, Artificial Sequence
  Synthetic oligonucleotide
aatgagccgagatggtgcc

SEQ ID NO: 422 - DNA, 19 bp, Artificial Sequence
  Synthetic oligonucleotide
ccgagatggtgccactgca

SEQ ID NO: 423 - DNA, 19 bp, Artificial Sequence
  Synthetic oligonucleotide
gagatggtgccactgcact

SEQ ID NO: 424 - DNA, 19 bp, Artificial Sequence
  Synthetic oligonucleotide
agatggtgccactgcactc

SEQ ID NO: 425 - DNA, 19 bp, Artificial Sequence
  Synthetic oligonucleotide
atggtgccactgcactctg

SEQ ID NO: 426 - DNA, 19 bp, Artificial Sequence
  Synthetic oligonucleotide
ccactgcactctgacgaca

SEQ ID NO: 427 - DNA, 19 bp, Artificial Sequence
  Synthetic oligonucleotide
cactgcactctgacgacag

SEQ ID NO: 428 - DNA, 19 bp, Artificial Sequence
  Synthetic oligonucleotide
tctgacgacagagcgagac

SEQ ID NO: 429 - DNA, 19 bp, Artificial Sequence
  Synthetic oligonucleotide
ctgacgacagagcgagact

SEQ ID NO: 430 - DNA, 19 bp, Artificial Sequence
  Synthetic oligonucleotide
gagactccgtctcaaaaca

SEQ ID NO: 431 - DNA, 19 bp, Artificial Sequence
  Synthetic oligonucleotide
agactccgtctcaaaacaa

SEQ ID NO: 432 - DNA, 19 bp, Artificial Sequence
  Synthetic oligonucleotide
tctagtgtttaaggatctg

SEQ ID NO: 433 - DNA, 19 bp, Artificial Sequence
  Synthetic oligonucleotide
tagtgtttaaggatctgcc

SEQ ID NO: 434 - DNA, 19 bp, Artificial Sequence
  Synthetic oligonucleotide
tgtttaaggatctgccttc

SEQ ID NO: 435 - DNA, 19 bp, Artificial Sequence
  Synthetic oligonucleotide
ggatctgccttccttcctg

SEQ ID NO: 436 - DNA, 19 bp, Artificial Sequence
  Synthetic oligonucleotide
ttgtctttccttgtttgtc

SEQ ID NO: 437 - DNA, 19 bp, Artificial Sequence
  Synthetic oligonucleotide
gtctttccttgtttgtctt

SEQ ID NO: 438 - DNA, 19 bp, Artificial Sequence
  Synthetic oligonucleotide
caagcaggttttaaattcc

SEQ ID NO: 439 - DNA, 19 bp, Artificial Sequence
  Synthetic oligonucleotide
gcaggttttaaattcctag

SEQ ID NO: 440 - DNA, 19 bp, Artificial Sequence
  Synthetic oligonucleotide
acatttacttttccaaggg

SEQ ID NO: 441 - DNA, 19 bp, Artificial Sequence
  Synthetic oligonucleotide
acactggagttcgagacga

SEQ ID NO: 442 - DNA, 19 bp, Artificial Sequence
  Synthetic oligonucleotide
ctggagttcgagacgaggc

SEQ ID NO: 443 - DNA, 19 bp, Artificial Sequence
  Synthetic oligonucleotide
ttcgagacgaggcctaagc

SEQ ID NO: 444 - DNA, 19 bp, Artificial Sequence
  Synthetic oligonucleotide
gagacgaggcctaagcaac

SEQ ID NO: 445 - DNA, 19 bp, Artificial Sequence
  Synthetic oligonucleotide
agacgaggcctaagcaaca

SEQ ID NO: 446 - DNA, 19 bp, Artificial Sequence
  Synthetic oligonucleotide
gacgaggcctaagcaacat

SEQ ID NO: 447 - DNA, 19 bp, Artificial Sequence
  Synthetic oligonucleotide
cctaagcaacatgccgaaa

SEQ ID NO: 448 - DNA, 19 bp, Artificial Sequence
  Synthetic oligonucleotide
ctaagcaacatgccgaaac

SEQ ID NO: 449 - DNA, 19 bp, Artificial Sequence
  Synthetic oligonucleotide
taagcaacatgccgaaacc

SEQ ID NO: 450 - DNA, 19 bp, Artificial Sequence
  Synthetic oligonucleotide
tggtggcgcacgcctatag

SEQ ID NO: 451 - DNA, 19 bp, Artificial Sequence
  Synthetic oligonucleotide
ggtggcgcacgcctatagt

SEQ ID NO: 452 - DNA, 19 bp, Artificial Sequence
  Synthetic oligonucleotide
ctatagtcctagctactgg

SEQ ID NO: 453 - DNA, 19 bp, Artificial Sequence
  Synthetic oligonucleotide
tatagtcctagctactggg

SEQ ID NO: 454 - DNA, 19 bp, Artificial Sequence
  Synthetic oligonucleotide
tgaggtgggaggatcgctt

SEQ ID NO: 455 - DNA, 19 bp, Artificial Sequence
  Synthetic oligonucleotide
ctgcagtgagccgagatcg

SEQ ID NO: 456 - DNA, 19 bp, Artificial Sequence
  Synthetic oligonucleotide
tgcagtgagccgagatcgc

SEQ ID NO: 457 - DNA, 19 bp, Artificial Sequence
  Synthetic oligonucleotide
tgcactccagcctgagcga

SEQ ID NO: 458 - DNA, 19 bp, Artificial Sequence
  Synthetic oligonucleotide
cactccagcctgagcgaca

SEQ ID NO: 459 - DNA, 19 bp, Artificial Sequence
  Synthetic oligonucleotide
acagggcgaggctctgtct

SEQ ID NO: 460 - DNA, 19 bp, Artificial Sequence
  Synthetic oligonucleotide
gggcgaggctctgtctcaa

SEQ ID NO: 461 - DNA, 19 bp, Artificial Sequence
  Synthetic oligonucleotide
ggcgaggctctgtctcaaa

SEQ ID NO: 462 - DNA, 19 bp, Artificial Sequence
  Synthetic oligonucleotide
gcgaggctctgtctcaaaa

SEQ ID NO: 463 - DNA, 19 bp, Artificial Sequence
  Synthetic oligonucleotide
aggctctgtctcaaaacaa

SEQ ID NO: 464 - DNA, 19 bp, Artificial Sequence
  Synthetic oligonucleotide
ggctctgtctcaaaacaaa

SEQ ID NO: 465 - DNA, 19 bp, Artificial Sequence
  Synthetic oligonucleotide
gctctgtctcaaaacaaac

SEQ ID NO: 466 - DNA, 19 bp, Artificial Sequence
  Synthetic oligonucleotide
ctctgtctcaaaacaaaca

SEQ ID NO: 467 - DNA, 19 bp, Artificial Sequence
  Synthetic oligonucleotide
aacacagtgaaatgaaagg

SEQ ID NO: 468 - DNA, 19 bp, Artificial Sequence
  Synthetic oligonucleotide
acacagtgaaatgaaagga

SEQ ID NO: 469 - DNA, 19 bp, Artificial Sequence
  Synthetic oligonucleotide
cagtgaaatgaaaggattg

SEQ ID NO: 470 - DNA, 19 bp, Artificial Sequence
  Synthetic oligonucleotide
gtgaaatgaaaggattgag

SEQ ID NO: 471 - DNA, 19 bp, Artificial Sequence
  Synthetic oligonucleotide
gaaatgaaaggattgagag

SEQ ID NO: 472 - DNA, 159 bp, Homo sapiens
agtcgcactctgtcactcaggctggagtgcagtggcgtgatcttggctcactgcaacctccgcctcccgagttcaagtgattctcctggctcagcctcccaagcagctgtcattacaggcctgcaccaccacacccggctgatttttgtatttttagga

SEQ ID NO: 473 - DNA, 82 bp, Homo sapiens
aatactggaggcccggtgtggtggctcacacctgtaatcccagcactttgggaggccgaggcggtcggattacgaggtcagg

SEQ ID NO: 474 - DNA, 815 bp, Homo sapiens
ctggccaacatggtgaaaccccatctttactaaaaatacaaaaattagccgggtgtggtggtgggcgcctgtaatcccagctactcggggggctgaggcagaattgcttgaacctgggaggcagaggttgcagtgagctgagatcacgccactgcattccagcctgggtgacagagcaatactctgtcgcaaaaaaaaaaaagaatactggaggctgggcgaggtggctcacacctgtaatcccagcattttgggatgccagaggcgggcggaatatcttgagctcaggagttcgagaccagcctacacaatatgctccaaacgccgcctctacaaaacatacagaaactagccgggtgtggtggcgtgcccctgtggtcctagctacttgggaggttgaggcgggaggatcgcttgagctcgggaggtcgaggctgcaatgagccgagatggtgccactgcactctgacgacagagcgagactccgtctcaaaacaaacaacaaataaggttgggggatcaaatatcttctagtgtttaaggatctgccttccttcctgcccccatgtttgtctttccttgtttgtctttatatagatcaagcaggttttaaattcctagtaggagcttacatttacttttccaagggggagggggaataaatatctacacacacacacacacacacacacacacacacacactggagttcgagacgaggcctaagcaacatgccgaaaccccgtctctactaaatacaaaaaatagctgagcttggtggcgcacgcctatagtcctagctactggggaggctg

SEQ ID NO: 475 - DNA, 108 bp, Homo sapiens
ctgcagtgagccgagatcgcgccgctgcactccagcctgagcgacagggcgaggctctgtctcaaaacaaacaaacaaaaaaaaaaggaaaggaaatataacacagtg

SEQ ID NO: 476 - DNA, 21 bp, Artificial Sequence
  Synthetic oligonucleotide
agacgaggccuaagcaacatt

SEQ ID NO: 477 - DNA, 21 bp, Artificial Sequence
  Synthetic oligonucleotide
uguugcuuaggccucgucutt

SEQ ID NO: 478 - DNA, 21 bp, Artificial Sequence
  Synthetic oligonucleotide
uuguacacuuggucaacautt

SEQ ID NO: 479 - DNA, 21 bp, Artificial Sequence
  Synthetic oligonucleotide
auguugaccaaguguacaatt

SEQ ID NO: 480 - DNA, 21 bp, Artificial Sequence
  Synthetic oligonucleotide
cacuggaguucgagacgagtt

SEQ ID NO: 481 - DNA, 21 bp, Artificial Sequence
  Synthetic oligonucleotide
cucgucucgaacuccagugtt

SEQ ID NO: 482 - DNA, 21 bp, Artificial Sequence
  Synthetic oligonucleotide
ggugacauuugugaaacuutt

SEQ ID NO: 483 - DNA, 21 bp, Artificial Sequence
  Synthetic oligonucleotide
aaguuucacaaaugucacctt

SEQ ID NO: 484 - DNA, 21 bp, Artificial Sequence
  Synthetic oligonucleotide
acuacugagugacaguagatt

SEQ ID NO: 485 - DNA, 21 bp, Artificial Sequence
  Synthetic oligonucleotide
ucuacugucacucaguagutt

SEQ ID NO: 486 - RNA, 18 nt, Artificial Sequence
  Synthetic oligonucleotide
  modified_base (1)..(18): 1,2,3,4,5,6,7,8,9,10,11,12,13,14,15,16,17, and 18 = “2′-O-methoxyethyl”
  modified_base (1)..(17): 1,2,3,4,5,6,7,8,9,10,11,12,13,14,15,16,and 17= “phosphorothioate backbone modification”
  modified_base (2)..(2): 5′ methyl cytosine
  modified_base (4)..(4): 5′ methyl cytosine
  modified_base (8)..(8): 5′ methyl cytosine
  modified_base (15)..(15): 5′ methyl cytosine
ucacuuucauaaugcugg

SEQ ID NO: 487 - DNA, 25 bp, Artificial Sequence
  Synthetic oligonucleotide primer
tcatactggctattatatgggtttt

SEQ ID NO: 488 - RNA, 18 nt, Artificial Sequence
  Synthetic oligonucleotide
  modified_base (1)..(17): 1,3,5,7,13,15, and 17 = “2′-O-methyl”
  modified_base (1)..(2): phosphorothioate  backbone modification
  modified_base (2)..(18): 2,4,6,8,9,10,11,12,14,16,and 18 = “2′-fluoro”
  modified_base (16)..(17): phosphorothioate  backbone modification
gacgaggccuaagcaaca

SEQ ID NO: 489 - DNA, 21 bp, Artificial Sequence
  Synthetic oligonucleotide primer
tgctctatgccagcatttctc

SEQ ID NO: 490 - DNA, 26 bp, Artificial Sequence
  Synthetic oligonucleotide primer
gctattatatggaaatgctggcatag

SEQ ID NO: 491 - DNA, 24 bp, Artificial Sequence
  Synthetic oligonucleotide primer
ttccagatctgtctgatcgtttct

SEQ ID NO: 492 - DNA, 22 bp, Artificial Sequence
  Synthetic oligonucleotide primer
tgctcacccaccaacaatttag

SEQ ID NO: 493 - DNA, 22 bp, Artificial Sequence
  Synthetic oligonucleotide primer
tctgctctgactttagcacctg

SEQ ID NO: 494 - DNA, 20 bp, Artificial Sequence
  Synthetic oligonucleotide primer
cccccaccacctcccatatg

SEQ ID NO: 495 - DNA, 25 bp, Artificial Sequence
  Synthetic oligonucleotide primer
cccttctcacagctcataaaattac

SEQ ID NO: 496 - RNA, 20 nt, Artificial Sequence
  Synthetic oligonucleotide
  modified_base (1)..(19): 1,3,5,7,9,11,13,15,17, and 19 = “2′-O-methyl”
  modified_base (1)..(19): 1,2,13,14,15,16,17,18, and 19 = “phosphorothioate backbone modification”
  modified_base (1)..(1): 5 (E)vinylphosphonate
  modified_base (2)..(20): 2,4,6,8,10,12,14,16,18, and 20 = “2′-fluoro”
uguugcuuaggccucgucuc

SEQ ID NO: 497 - DNA, 20 bp, Artificial Sequence
  Synthetic oligonucleotide primer
gctctggaattgtaccgcag

SEQ ID NO: 498 - DNA, 20 bp, Artificial Sequence
  Synthetic oligonucleotide primer
ctgcagcaaatcgcttggga